(19) United States
(12) Patent Application Publication
(10) Pub. No.: US 2008/0227797 A1
(43) Pub. Date: Sep. 18, 2008

(54) Pyridopyrimidine Derivatives as Pde4 Inhibitors for the Treatment of Inflammatory and Immune Diseases

(75) Inventors: Annea Lisius, Lund (SE); Grigorios Nikitidis, Lund (SE); Peter Sjo, Lund (SE)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/994,572

(22) PCT Filed: Jul. 3, 2006
(86) PCT No.: PCT/SE2006/000826

§ 371 Date: Jan. 3, 2008

(30) Foreign Application Priority Data
Jul. 4, 2005 (SE) 0501564-9
Mar. 8, 2006 (SE) 0600516-9

(51) Int. Cl. C07D 487/04; A61K 31/519; A61P 37/00

(52) U.S. Cl. 514/264.1; 544/279

(57) ABSTRACT

The present invention provides a compound of a formula (I): [Chemical formula should be inserted here. Please see paper copy] wherein the variables are defined herein; to a process for preparing such a compound; and to the use of such a compound in the treatment of a PDE4 mediated disease state.

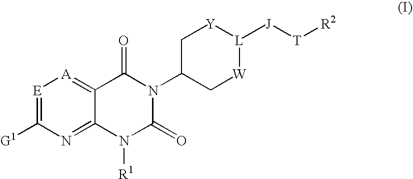

The present invention concerns pyridopyrimidine derivatives having pharmaceutical activity, to processes for preparing such derivatives, to pharmaceutical compositions comprising such derivatives and to the use of such derivatives as active therapeutic agents.

Pharmaceutically active pyridopyrimidine derivatives are disclosed in EP-A-0260817, WO 98/02162, WO 93/19068 and WO 0045800.

Phosphodiesterases (PDEs) work by converting cAMP or cGMP to AMP and GMP, or the inactive nucleotide forms incapable of activating downstream signalling pathways. The inhibition of PDEs leads to the accumulation of cAMP or cGMP, and subsequent activation of downstream pathways. PDEs comprise a large family of second messengers with 11 families and over 50 isoforms. In addition splice variants have been described for each isoform. The PDEs can be cAMP-specific (PDE4, 7, 8, 10), cGMP is specific (PDE5, 6, 9) or have dual specificity (PDE1, 2, 3, 11).

cAMP is generated from ATP at the inner leaflet of the plasma membrane through the action of GPCR-regulated adenylate cyclase. Once cAMP is generated, the only way to terminate the signal is through phosphodiesterase action, degrading cAMP into 5′-AMP. Increased concentrations of cAMP are translated into cellular responses mainly by activation of cAMP-dependent protein kinase (PKA). The specific activity of PKA is in part regulated by the sub-cellular localization of PKA, which limits the phosphorylation of PKA to substrates in its near vicinity. The downstream events caused by activation of PKA appear poorly elucidated and involve many components in the initiation of signalling cascades. PDE4s have been shown to have abundant roles in regulating cell desensitisation, adaptation, signal cross-talk, cAMP compartmentalization and feedback loops, and are major regulators of cAMP homeostasis.

The physiological role implicated for elevated cAMP levels include: 1) broad suppression the activity of many immunocompetent cells; 2) induction of airway smooth muscle relaxation; 3) suppression of smooth muscle mitogenesis; and, 4) has beneficial modulatory effects on the activity of pulmonary nerves.

PDE4 has been found to be the predominant cAMP metabolising isozyme family in immune and inflammatory cells and, along with the PDE3 family, a major contributor to cAMP metabolism in airway smooth muscle.

Over the last two decades significant attention has been devoted into the development of PDE4 selective inhibitors for the treatment of inflammatory and immune disorders including asthma, rhinitis, bronchitis, COPD, arthritis and psoriasis. A number of compounds (for example rolipram, tibenelast and denbufylline) have been reported to have impressive effects in animal models of inflammation, especially pulmonary inflammation.

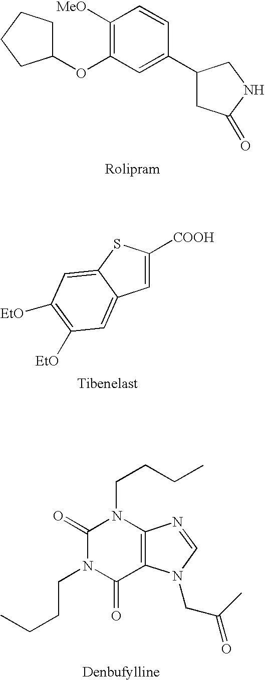

Unfortunately the clinical utility of these inhibitors has been limited by PDE4 related side-effects, including nausea, vomiting and gastric acid secretion. Recently a second generation of PDE4 inhibitors (for example cilomilast, roflumilast and AWD 12-281) has been described having significantly reduced risk of emetic side effects in animal models of emesis, thus providing the potential for an increased therapeutic ratio.

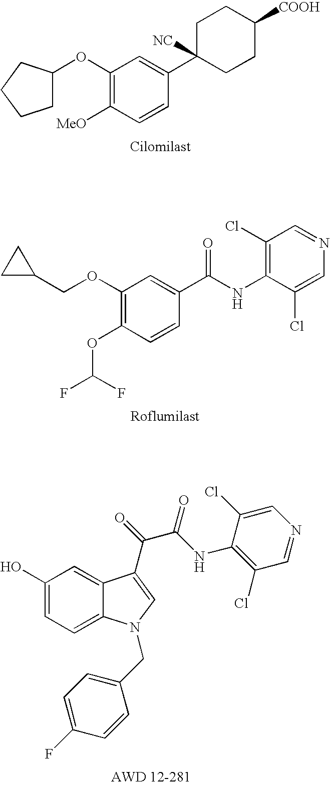

The present invention discloses novel pyridopyrimidine derivatives that are inhibitors of human PDE4 and are thereby useful in therapy.

The present invention provides a compound of formula (I):

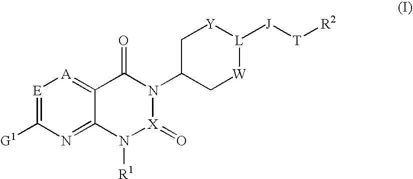

wherein:

### A is N or CA¹;

### E is N or CE¹;

### T is C(O) or S(O)₂;

### X is C or S;

W is (CH₂)ₙ;
Y is (CH₂)ₚ;
n and p are, independently 0 or 1;

### L is CH or N;

when L is CH then J is NH; and when L is N then J is absent and T is bonded directly to L;
R¹ is aryl or heteroaryl, each of which is optionally substituted by halogen, cyano, hydroxy, C₁₋₄ alkyl, C₁₋₄ alkoxy, CF₃, OCF₃, C₁₋₄ alkylthio, S(O)(C₁₋₄ alkyl), S(O)₂(C₁₋₄ alkyl) or C(O)₂(C₁₋₄ alkyl);
R² is C₁₋₆ alkyl (optionally substituted by hydroxyl, C₁₋₆ alkoxy, heterocyclyl (optionally substituted by C₁₋₆ alkyl), aryl, heteroaryl, C₃₋₇ cycloalkyl, CO₂H, CO₂(C₁₋₆ alkyl) or NHC(O)R³), C₁₋₆ alkoxy, C₃₋₆ cycloalkyl (optionally substituted by hydroxyl or C₁₋₆ alkyl), heterocyclyl (optionally substituted by C₁₋₆ alkyl), aryl or heteroaryl;
R³ is C₁₋₆ alkyl or phenyl;
the foregoing phenyl, aryl and heteroaryl moieties of R² and R³ are, independently, optionally substituted by: halogen, cyano, nitro, hydroxy, S(O)_{q}R⁴, OC(O)NR⁵R⁶, NR⁷R⁵, NR⁹C(O)R¹⁰, NR¹¹C(O)NR¹²R¹³, S(O)₂NR¹⁴R¹⁵, NR¹⁶S(O)₂R¹⁷, C(O)NR¹⁸R¹⁹, C(O)R²⁰, CO₂R²¹, NR²²CO₂R²³, C₁₋₆ alkyl, C₁₋₆ hydroxyalkyl, C₁₋₆ haloalkyl, C₁₋₆ alkoxy(C₁₋₆)allyl, di(C₁₋₆)alkylamino(C₁₋₆)alkyl, C₁₋₆ alkoxy, C₁₋₆ haloalkoxy, C₁₋₆ alkoxy(C₁₋₆)alkoxy, C₁₋₆ allylthio, C₂₋₆ alkenyl, C₂₋₆ alkynyl, C₃₋₁₀ cycloalkyl (itself optionally substituted by C₁₋₄ alkyl or oxo), methylenedioxy, difluoromethylenedioxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halogen, hydroxy, nitro, S(O)ᵣ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ allyl), CF₃ or OCF₃;
A¹, E¹ and G¹ are, independently, hydrogen, halogen, cyano, hydroxy, C₁₋₄ alkyl, C₁₋₄ alkoxy, CF₃ or OCF₃; t
q and r are, independently, 0, 1 or 2;
R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are, independently, C₁₋₆ alkyl {optionally substituted by halogen, hydroxy or C₁₋₆ alkoxy}, CH₂(C₂₋₆ alkenyl), phenyl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃} or heteroaryl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ allyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}; R⁵, R⁶, R⁷, R⁸, R⁹, R¹⁰, R¹¹, R¹², R¹³, R¹⁴, R¹⁵, R¹⁶, R¹⁸, R¹⁹, R²⁰, R²¹, R²² and R²³ can also be hydrogen; or a N-oxide thereof; or a pharmaceutically acceptable salt thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, dihydrochloride, hydrobromide, phosphate, sulfate, acetate, diacetate, fumarate, maleate, malonate, succinate, tartrate, citrate, oxalate, methanesulfonate or p-toluenesulfonate. An alternative acid addition salt is a trifluoroacetate salt.

The compounds of the invention may exist as solvates (such as hydrates) and the present invention covers all such solvates.

Halogen includes fluorine, chlorine, bromine and iodine. Halogen is, for example, fluorine or chlorine.

Alkyl moieties are straight or branched chain and are, for example, methyl, ethyl, n-propyl, iso-propyl or tert-butyl. Haloalkyl is, for example C₂F₅, CF₃ or CHF₂. Alkoxy is, for example, methoxy or ethoxy; and haloalkoxy is, for example OCF₃ or OCHF₂.

Alkenyl is, for example, vinyl or prop-2-enyl. Alkynyl is, for example, propargyl.

Cycloalkyl is, for example, cyclopropyl, cyclobutyl, cyclopentyl or cyclohexyl. Cycloalkyloxy is, for example, cyclopropyloxy, cyclopentyloxy or cyclohexyloxy. Cycloalkylalkoxy is, for example, (cyclopropyl)methoxy or 2-(cyclopropyl)ethoxy.

Heterocyclyl is a non-aromatic 5- or 6-membered ring, comprising at least one heteroatom selected from the group comprising nitrogen, oxygen and sulphur. Heterocyclyl is, for example, pyrrolidinyl, piperidinyl or morpholinyl.

Hydroxyalkyl is, for example, CH₂OH; C₁₋₆ alkoxy(C₁₋₆)alkyl is, for example CH₃OCH₂; and, C₁₋₆ alkoxy(C₁₋₆)alkoxy is, for example, CH₃OCH₂O. Dialkylaminoalkyl is, for example (CH₃)₂NCH₂ or (CH₃)(CH₃CH₂)NCH₂. Heteroaryl is, for example, an aromatic 5- or 6-membered ring, optionally fused to one or more other rings, comprising at least one heteroatom selected from the group comprising nitrogen, oxygen and sulphur; or an N-oxide thereof, or an S-oxide or S-dioxide thereof. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,4]-triazolyl, [1,2,3]-triazolyl, pyridinyl, pyrimidinyl, pyrazinyl, pyridazinyl, indolyl, benzo[b]furyl (also known as benzfuryl), benz[b]thienyl (also known as benzthienyl or benzthiophenyl), indazolyl, benzimidazolyl, benztriazolyl, benzoxazolyl, benzthiazolyl (for example 1,3-benzthiazolyl), 1,2,3-benzothiadiazolyl, an imidazopyridinyl (such as imidazo[1,2a]pyridinyl), thieno[3,2-b]pyridin-6-yl, 1,2,3-benzoxadiazolyl (also known as benzo[1,2,3]thiadiazolyl), 2,1,3-benzothiadiazolyl, benzofurazan (also known as 2,1,3-benzoxadiazolyl), quinoxalinyl, a pyrazolopyridine (for example 1H-pyrazolo[3,4-b]pyridinyl or pyrazolo[1,5-a]pyridinyl), an imidazopyridine (for example imidazo[1,2-a]pyridinyl), a dihydropyrido[2,3-d]pyrimidine (for example 1,4-dihydropyrido[2,3-d]pyrimidinyl), quinolinyl, isoquinolinyl or a naphthyridinyl (for example [1,6]naphthyridinyl or [1,8]naphthyridinyl); or an N-oxide thereof, or an S-oxide or S-dioxide thereof. In another aspect heteroaryl is, for example, 1,2,3-thiadiazolyl, 1H-pyrrolo[2,3-b]pyridinyl, thieno[2,3-b]pyridinyl, thieno[2,3-b]pyrazinyl, [1,2,4]triazolo[1,5-a]pyrimidinyl or 6,7-dihydro-5H-[1,3]thiazolo[3,2-a]pyrimidinyl.

In one aspect the present invention provides a compound of formula (I) wherein: A is N or CA¹; E is N or CE¹; T is C(O) or S(O)₂; X is C or S; W is (CH₂)ₙ; Y is (CH₂)ₚ; n and p are, independently 0 or 1; L is CH or N; when L is CH then J is NH; and when L is N then J is absent and T is bonded directly to L; R¹ is aryl or heteroaryl, each of which is optionally substituted by halogen, cyano, hydroxy, C₁₋₄ allyl, C₁₋₄ alkoxy, CF₃, OCF₃, C₁₋₄ alkylthio, S(O)(C₁₋₄ alkyl), S(O)₂(C₁₋₄ alkyl) or C(O)₂(C₁₋₄ alkyl); R² is C₁₋₆ alkyl (optionally substituted by hydroxyl, C₁₋₆ alkoxy, aryl, heteroaryl, C₃₋₇ cycloalkyl, CO₂H, CO₂(C₁₋₆ alkyl) or NHC(O)R³), C₁₋₆ alkoxy, C₃₋₆ cycloalkyl (optionally substituted by hydroxyl or C₁₋₆ alkyl), heterocyclyl (optionally substituted by C₁₋₆ alkyl), aryl or heteroaryl; R³ is C₁₋₆ alkyl or phenyl; the foregoing phenyl, aryl and heteroaryl moieties of R² and R³ are, independently, optionally substituted by: halogen, cyano, nitro, hydroxy, S(O)_{q}R⁴, OC(O)NR⁵R⁶, NR⁷R⁸, NR⁹C(O)R¹⁰, NR¹¹C(O)NR¹²R¹³, S(O)₂NR¹⁴R¹⁵, NR¹⁶S(O)₂R¹⁷, C(O)NR¹⁸R¹⁹, C(O)R²⁰, COR²¹, NR²²COR²³, C₁₋₆ alkyl, C₁₋₆ hydroxyalkyl, C₁₋₆ haloalkyl, C₁₋₆ alkoxy(C₁₋₆)alkyl, di(C₁₋₆)alkylamino(C₁₋₆)alkyl, C₁₋₆ alkoxy, C₁₋₆ haloalkoxy, C₁₋₆ alkoxy(C₁₋₆)alkoxy, C₁₋₆ alkylthio, C₂₋₆ alkenyl, C₂₋₆ alkynyl, C₃₋₁₀ cycloalkyl (itself optionally substituted by C₁₋₄ alkyl or oxo), methylenedioxy, difluoromethylenedioxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halogen, hydroxy, nitro, S(O)ᵣ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; A¹, E¹ and G¹ are, independently, hydrogen, halogen, cyano, hydroxy, C₁₋₄ alkyl, C₁₋₄ alkoxy, CF₃ or OCF₃; q and r are, independently, 0, 1 or 2; R⁴, R⁵, R⁶, R⁷, R⁸, R⁹, R¹⁰, R¹¹R¹², R¹³, R¹⁴, R¹⁵, R¹⁶, R¹⁷, R¹⁸, R¹⁹, R²⁰, R²¹, R²² and R²³ are, independently, C₁₋₆ alkyl {optionally substituted by halogen, hydroxy or C₁₋₆ alkoxy}, CH₂(C₂₋₆ alkenyl), phenyl {itself optionally substituted by halogen, hydroxy, nitro, NH₂, NH(C₁₋₄ alkyl), N(C₁₋₄ alkyl)₂, S(O)₂(C₁₋₄ allyl), S(O)₂NH₂, S(O)₂NH(C₁₋₄ alkyl), S(O)₂N(C₁₋₄ allyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃} or heteroaryl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}; R⁵, R⁶, R⁷, R⁸, R⁹, R¹⁰, R¹¹, R¹², R¹³, R¹⁴, R¹⁵, R¹⁶, R¹⁸, R¹⁹, R²⁰, R²¹, R²² and R²³ can also be hydrogen; or a N-oxide thereof; or a pharmaceutically acceptable salt thereof.

In another aspect the present invention provides a compound of formula (I) wherein: A is N or CA¹; E is N or CE¹; T is C(O) or S(O)₂; X is C or S; W is (CH₂)ₙ; Y is (CH₂)ₚ; n and p are, independently 0 or 1; L is CH or N; when L is CH then J is NH; and when L is N then J is absent and T is bonded directly to L; R¹ is aryl or heteroaryl, each of which is optionally substituted by halogen, cyano, hydroxy, C₁₋₄ alkyl, C₁₋₄ alkoxy, CF₃, OCF₃, C₁₋₄ alkylthio, S(O)(C₁₋₄ alkyl), S(O)₂(C₁₋₄ alkyl) or C(O)₂(C₁₋₄ alkyl); R² is C₁₋₆ alkyl (optionally substituted by hydroxyl, aryl, heteroaryl, C₃₋₇ cycloalkyl, CO₂H, CO₂(C₁₋₆ alkyl) or NHC(O)R³), C₁₋₆ alkoxy, aryl or heteroaryl; R³ is C₁₋₆ alkyl or phenyl; the foregoing phenyl, aryl and heteroaryl moieties of R² and R³ are, independently, optionally substituted by: halogen, cyano, nitro, hydroxy, S(O)_{q}R⁴, OC(O)NR⁵R⁶, NR⁷R⁸, NR⁹C(O)R¹⁰, NR¹¹C(O)NR¹²R¹³, S(O)₂NR¹⁴R¹⁵, NR¹⁶S(O)₂R¹⁷, C(O)NR¹⁸R¹⁹, C(O)R²⁰, CO₂R²¹, NR²²CO₂R²³, C₁₋₆ alkyl, C₁₋₆ hydroxyalkyl, C₁₋₆ haloalkyl, C₁₋₆ alkoxy(C₁₋₆)alkyl, C₁₋₆ alkoxy, C₁₋₆ haloalkoxy, C₁₋₆ alkoxy(C₁₋₆)alkoxy, C₁₋₆ alkylthio, C₂₋₆ alkenyl, C₂₋₆ alkynyl, C₃₋₁₀ cycloalkyl (itself optionally substituted by C₁₋₄ alkyl or oxo), methylenedioxy, difluoromethylenedioxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halogen, hydroxy, nitro, S(O)ᵣ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; A¹, E¹ and G¹ are, independently, hydrogen, halogen, cyano, hydroxy, C₁₋₄ alkyl, C₁₋₄ alkoxy, CF₃ or OCF₃; q and r are, independently, 0, 1 or 2; R⁴, R⁵, R⁶, R⁷, R⁸, R⁹, R¹⁰, R¹¹, R¹², R¹³, R¹⁴, R¹⁵, R¹⁶, R¹⁷, R¹⁸R¹⁹, R²⁰, R²¹, R² and R²³ are, independently, C₁₋₆ alkyl {optionally substituted by halogen, hydroxy or C₁₋₆ alkoxy}, CH₂(C₂₋₆ alkenyl), phenyl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃} or heteroaryl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}; R⁵, R⁶, R⁷, R⁸, R⁹, R¹⁰, R¹¹, R¹², R¹³, R¹⁴, R¹⁵, R¹⁶, R¹⁸, R¹⁹, R²⁰, R²¹, R²² and R²³ can also be hydrogen; or a N-oxide thereof; or a pharmaceutically acceptable salt thereof.

In yet another aspect the present invention provides a compound of formula (I) wherein A is CA¹; E is CE¹; and A¹, E¹ and G¹ are, independently, hydrogen or halogen (for example fluoro).

In a further aspect the present invention provides a compound of formula (I) wherein A is CA¹. A¹ is, for example, hydrogen.

In a still further aspect the present invention provides a compound of formula (I) wherein E is CE¹. E¹ is, for example, halogen (such as fluoro).

In another aspect the present invention provides a compound of formula (I) wherein G¹ is hydrogen.

In yet another aspect the present invention provides a compound of formula (I) wherein X is C.

In a further aspect the present invention provides a compound of formula (I) wherein n and p are both 1.

In a still further aspect the present invention provides a compound of formula (I) wherein L is CH.

In another aspect the present invention provides a compound of formula (I) wherein J is NH.

In yet another aspect the present invention provides a compound of formula (I) wherein T is C(O).

In a still further aspect the present invention provides a compound of formula (I) wherein R¹ is aryl (for example phenyl) optionally substituted by halogen, C₁₋₄ alkylthio, S(O)₂(C₁₋₄alkyl) or C(O)₂(C₁₋₄ alkyl). In a further aspect the present invention provides a compound of formula (I) wherein R¹ is phenyl optionally substituted by halogen (for example fluoro) or C₁₋₄ alkylthio (for example SCH₃). In a still further aspect the present invention provides a compound of formula (I) wherein R¹ is phenyl optionally substituted by fluoro (for example by 2 fluoro).

In another aspect the present invention provides a compound of formula (I) wherein R² is C₁₋₆ alkyl {optionally substituted by hydroxy, phenyl (itself optionally substituted by Hydroxyl), C₃₋₇ cycloalkyl, CO₂(C₁₋₄ alkyl) or NHC(O)R³}, phenyl {optionally substituted by halogen, hydroxyl, C₁₋₄ alkyl, C₁₋₄ alkoxy, CO₂(C₁₋₄ alkyl) or C₂₋₄ alkynyl} or heteroaryl {optionally substituted by halogen, hydroxyl, C₁₋₄ alkyl, C₁₋₄ alkoxy, CO₂(C₁₋₄ alkyl) or C₂₋₄ alkynyl}; wherein R³ is phenyl optionally substituted by hydroxyl. Heteroaryl is, for example, indolyl, quinolinyl, benzpyrazolyl, imidazo[1,2-a]pyridinyl, pyrazolo[1,5-a]pyridinyl or pyrrolo[2,3-b]pyridinyl.

In a further aspect the present invention provides a compound of formula (I) wherein R² is C₁₋₆ alkyl (optionally substituted by hydroxy, aryl, C₃₋₇ cycloalkyl, CO₂(C₁₋₆ alkyl) or NHC(O)R³), aryl or heteroaryl (for example pyridyl, benzimidazolyl or 1,4-dihydropyrido[2,3-d]pyrimidinyl); and R³ is phenyl. Aryl is, for example, phenyl. In a still further aspect the present invention provides a compound of formula (I) wherein the foregoing phenyl, aryl and heteroaryl moieties of R² and R³ are, independently, optionally substituted by: halogen, hydroxy, CO₂(C₁₋₆ alkyl), C₁₋₆ alkyl, C₁₋₆ hydroxyalkyl or C₁₋₆ alkoxy.

Compounds of the invention are described in the Examples. Each of the compounds of the Examples is a further aspect of the present invention. Thus the present invention provides a compound:
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-5-(hydroxymethyl)benzamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-2-phenylacetamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxyacetamide;
5-chloro-N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-4-hydroxybenzamide;
methyl 2-[({cis-4-[1-(3,4-difluorophenyl)-6-fluoro-2,4-dioxo-1,4-dihydropyrido[2,3-d]pyrimidin-3(2H)-yl]cyclohexyl}amino)carbonyl]benzoate;
N-{cis-4-[1-(3,4-difluorophenyl)-6-fluoro-2,4-dioxo-1,4-dihydropyrido[2,3-d]pyrimidin-3(2H)-yl]cyclohexyl}-2-(4-hydroxyphenyl)acetamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-4-methylbenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-hydroxypyridine-2-carboxamide;
3-chloro-N-{cis-4-[1-(3,4-difluorophenyl)-6-fluoro-2,4-dioxo-1,4-dihydropyrido[2,3-d]pyrimidin-3(2H)-yl]cyclohexyl}-4-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-hydroxy-2-methylbenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-4-hydroxy-3-methoxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-hydroxy-4-methylbenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-6-methoxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-3-methoxybenzamide;
4-chloro-N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-4-methoxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-5-fluoro-2-hydroxybenzamide;
(2S)—N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-4-methylpentanamide;
(2R)-2-cyclohexyl-N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxyacetamide;
methyl 4-({cis-4-[1-(3,4-difluorophenyl)-6-fluoro-2,4-dioxo-1,4-dihydropyrido[2,3-d]pyrimidin-3(2H)-yl]cyclohexyl}amino)-4-oxobutanoate;
N-[2-({cis-4-[1-(3,4-difluorophenyl)-6-fluoro-2,4-dioxo-1,4-dihydropyrido[2,3-d]pyrimidin-3(2H)-yl]cyclohexyl}amino)-2-oxoethyl]-2-hydroxybenzamide;
methyl 4-[({cis-4-[1-(3,4-difluorophenyl)-6-fluoro-2,4-dioxo-1,4-dihydropyrido[2,3-d]pyrimidin-3(2H)-yl]cyclohexyl}amino)carbonyl]benzoate;
N-{cis-4-[1-(3,4-difluorophenyl)-6-fluoro-2,4-dioxo-1,4-dihydropyrido[2,3-d]pyrimidin-3(2H)-yl]cyclohexyl}-3-hydroxy-4-methoxybenzamide;
methyl 5-({cis-4-[1-(3,4-difluorophenyl)-6-fluoro-2,4-dioxo-1,4-dihydropyrido[2,3-d]pyrimidin-3(2H)-yl]cyclohexyl}amino)-5-oxopentanoate;
N-{cis-4-[1-(3,4-difluorophenyl)-6-fluoro-2,4-dioxo-1,4-dihydropyrido[2,3-d]pyrimidin-3(2H)-yl]cyclohexyl}-2-(3-hydroxyphenyl)acetamide;
methyl 3-[({cis-4-[1-(3,4-difluorophenyl)-6-fluoro-2,4-dioxo-1,4-dihydropyrido[2,3-d]pyrimidin-3(2H)-yl]cyclohexyl}amino)carbonyl]benzoate;
N-{cis-4-[1-(3,4-difluorophenyl)-6-fluoro-2,4-dioxo-1,4-dihydropyrido[2,3-d]pyrimidin-33(2H)-yl]cyclohexyl}-2-fluoro-6-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-(3-hydroxyphenyl)propanamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-indazole-3-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}imidazo[1,2-a]pyridine-2-carboxamide;
5-chloro-N-{cis-4-[6-fluoro-1-[3-(methylthio)phenyl]-2,4-dioxo-1,4-dihydropyrido[2,3-d]pyrimidin-3(2H)-yl]cyclohexyl}-2-hydroxybenzamide;
N-{cis-4-[6-fluoro-1-[3-(methylthio)phenyl]-2,4-dioxo-1,4-dihydropyrido[2,3-d]pyrimidin-33(2H)-yl]cyclohexyl}-2-hydroxy-5-methylbenzamide;
N-{cis-4-[6-fluoro-1-[3-(methylthio)phenyl]-2,4-dioxo-1,4-dihydropyrido[2,3-d]pyrimidin-3(2H)-yl]cyclohexyl}-2-hydroxy-5-(hydroxymethyl)benzamide;
(2R)—N-{cis-4-[6-fluoro-1-[3-(methylthio)phenyl]-2,4-dioxo-1,4-dihydropyrido[2,3-d]pyrimidin-3(2H)-yl]cyclohexyl}-2-hydroxy-2-phenylacetamide;
(2S)—N-{cis-4-[6-fluoro-1-[3-(methylthio)phenyl]-2,4-dioxo-1,4-dihydropyrido[2,3-d]pyrimidin-3(2H)-yl]cyclohexyl}-2-hydroxy-2-phenylacetamide;
N-{cis-4-[6-fluoro-1-[3-(methylthio)phenyl]-2,4-dioxo-1,4-dihydropyrido[2,3-d]pyrimidin-3(2H)-yl]cyclohexyl}-2-hydroxyacetamide;
5-chloro-N-{cis-4-[6-fluoro-1-[3-(methylsulfonyl)phenyl]-2,4-dioxo-1,4-dihydropyrido[2,3-d]pyrimidin-3(2H)-yl]cyclohexyl}-2-hydroxybenzamide;
N-{cis-4-[6-fluoro-1-[3-(methylsulfonyl)phenyl]-2,4-dioxo-1,4-dihydropyrido[2,3-d]pyrimidin-3(2H)-yl]cyclohexyl}-2-hydroxy-5-(hydroxymethyl)benzamide;
(2R)—N-{cis-4-[6-fluoro-1-[3-(methylsulfonyl)phenyl]-2,4-dioxo-1,4-dihydropyrido[2,3-d]pyrimidin-3(2H)-yl]cyclohexyl}-2-hydroxy-2-phenylacetamide;
(2S)—N-{cis-4-[6-fluoro-1-[3-(methylsulfonyl)phenyl]-2,4-dioxo-1,4-dihydropyrido[2,3-d]pyrimidin-3(2H)-yl]cyclohexyl}-2-hydroxy-2-phenylacetamide;
N-{cis-4-[6-fluoro-1-[3-(methylsulfonyl)phenyl]-2,4-dioxo-1,4-dihydropyrido[2,3-d]pyrimidin-3(2H)-yl]cyclohexyl}-2-hydroxyacetamide;
methyl 3-[6-fluoro-3-{cis-4-[(2-hydroxy-5-methylbenzoyl)amino]cyclohexyl}-2,4-dioxo-3,4-dihydropyrido[2,3-d]pyrimidin-1(2H)-yl]benzoate;
N-{cis-4-[1-(3,4-difluorophenyl)-6-fluoro-2,4-dioxo-1,4-dihydropyrido[2,3-d]pyrimidin-3(2H)-yl]cyclohexyl}methanesulfonamide;
N-{cis-4-[1-(3,4-difluorophenyl)-6-fluoro-2-oxido-4-oxo-1,4-dihydro-3H-pyrido[2,3-c][1,2,6]thiadiazin-3-yl]cyclohexyl}-2-hydroxy-5-methylbenzamide;
N-{cis-4-[1-(3,4-Difluorophenyl)-6-fluoro-2,4-dioxo-1,4-dihydropyrido[2,3-d]pyrimidin-3(2H)-yl]cyclohexyl}cyclopropanecarboxamide;
Methyl 3-[6-fluoro-3-{cis-4-[(1-methyl-L-prolyl)amino]cyclohexyl}-2,4-dioxo-3,4-dihydropyrido[2,3-d]pyrimidin-1(2H)-yl]benzoate;
methyl 5-[({cis-4-[1-(3,4-difluorophenyl)-6-fluoro-2,4-dioxo-1,4-dihydropyrido[2,3-d]pyrimidin-3(2H)-yl]cyclohexyl}amino)carbonyl]pyridine-2-carboxylate;
N-{cis-4-[1-(3,4-difluorophenyl)-6-fluoro-2,4-dioxo-1,4-dihydropyrido[2,3-d]pyrimidin-3(2H)-yl]cyclohexyl}-4-fluoro-2-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-1-hydroxycyclopropanecarboxamide;
N-{cis-4-[1-(3,4-difluorophenyl)-6-fluoro-2,4-dioxo-1,4-dihydropyrido[2,3-d]pyrimidin-3(2H)-yl]cyclohexyl}quinoline-8-carboxamide;
N-{cis-4-[6-fluoro-1-[3-(methylthio)phenyl]-2,4-dioxo-1,4-dihydropyrido[2,3-d]pyrimidin-3(2H)-yl]cyclohexyl}-2-furamide;
N-{cis-4-[6-fluoro-1-[3-(methylthio)phenyl]-2,4-dioxo-1,4-dihydropyrido[2,3-d]pyrimidin-3(2H)-yl]cyclohexyl}cyclopropanecarboxamide;
N-{cis-4-[6-fluoro-1-[3-(methylthio)phenyl]-2,4-dioxo-1,4-dihydropyrido[2,3-d]pyrimidin-3(2H)-yl]cyclohexyl}propanamide;
N-{cis-4-[6-fluoro-1-[3-(methylthio)phenyl]-2,4-dioxo-1,4-dihydropyrido[2,3-d]pyrimidin-3(2H)-yl]cyclohexyl}benzamide;
N-{cis-4-[6-fluoro-1-[3-(methylthio)phenyl]-2,4-dioxo-1,4-dihydropyrido[2,3-d]pyrimidin-3(2H)-yl]cyclohexyl}cyclopentanecarboxamide;
N-{cis-4-[6-fluoro-1-[3-(methylthio)phenyl]-2,4-dioxo-1,4-dihydropyrido[2,3-d]pyrimidin-3 (2H)-yl]cyclohexyl}-2-hydroxy-2-methylpropanamide;
N-{cis-4-[6-fluoro-1-[3-(methylthio)phenyl]-2,4-dioxo-1,4-dihydropyrido[2,3-d]pyrimidin-3(2H)-yl]cyclohexyl}benzenesulfonamide;
methyl 3-[3-{cis-4-[(cyclobutylcarbonyl)amino]cyclohexyl}-6-fluoro-2,4-dioxo-3,4-dihydropyrido[2,3-d]pyrimidin-1(2H)-yl]benzoate;
methyl 3-[6-fluoro-3-[cis-4-(isobutyrylamino)cyclohexyl]-2,4-dioxo-3,4-dihydropyrido[2,3-d]pyrimidin-1(2H)-yl]benzoate;
methyl 3-[6-fluoro-3-{cis-4-[(3-methylbutanoyl)amino]cyclohexyl}-2,4-dioxo-3,4-dihydropyrido[2,3-d]pyrimidin-1(2H)-yl]benzoate;
methyl 3-[3-{cis-4-[(2,2-dimethylpropanoyl)amino]cyclohexyl}-6-fluoro-2,4-dioxo-3,4-dihydropyrido[2,3-d]pyrimidin-1(2H)-yl]benzoate;
methyl 3-[6-fluoro-3-{cis-4-[(methoxyacetyl)amino]cyclohexyl}-2,4-dioxo-3,4-dihydropyrido[2,3-d]pyrimidin-1(2H)-yl]benzoate;
methyl 3-[6-fluoro-2,4-dioxo-3-{cis-4-[(2-thienylcarbonyl)amino]cyclohexyl}-3,4-dihydropyrido[2,3-d]pyrimidin-1(2H)-yl]benzoate;
methyl 3-[3-[cis-4-(acetylamino)cyclohexyl]-6-fluoro-2,4-dioxo-3,4-dihydropyrido[2,3-d]pyrimidin-1(2H)-yl]benzoate;
methyl 3-[6-fluoro-2,4-dioxo-3-{cis-4-[(pyrazolo[1,5-a]pyridin-2-ylcarbonyl)amino]cyclohexyl}-3,4-dihydropyrido[2,3-d]pyrimidin-1(2H)-yl]benzoate;
methyl 3-[6-fluoro-3-{cis-4-[(imidazo[1,2-a]pyridin-2-ylcarbonyl)amino]-cyclohexyl}-2,4-dioxo-3,4-dihydropyrido[2,3-d]pyrimidin-1(2H)-yl]benzoate;
methyl 3-[3-{cis-4-[(cyclopropylcarbonyl)amino]cyclohexyl}-6-fluoro-2,4-dioxo-3,4-dihydropyrido[2,3-d]pyrimidin-1(2H)-yl]benzoate;
N-{cis-4-[6-fluoro-1-[3-(methylsulfonyl)phenyl]-2,4-dioxo-1,4-dihydropyrido[2,3-d]pyrimidin-3(2H)-yl]cyclohexyl}cyclopropanecarboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-5-methylbenzamide;
methyl 3-[6-fluoro-3-(cis-4-{[(1-methylpiperidin-4-yl)carbonyl]amino}cyclohexyl)-2,4-dioxo-3,4-dihydropyrido[2,3-d]pyrimidin-1(2H)-yl]benzoate;
methyl 3-[3-[cis-4-({5-[(dimethylamino)methyl]-2-furoyl}amino)cyclohexyl]-6-fluoro-2,4-dioxo-3,4-dihydropyrido[2,3-d]pyrimidin-1(2H)-yl]benzoate;
methyl 3-[3-{cis-4-[(cyclohexylcarbonyl)amino]cyclohexyl}-6-fluoro-2,4-dioxo-3,4-dihydropyrido[2,3-d]pyrimidin-1(2H)-yl]benzoate;
tert-butyl 4-[({2-[(3,4-difluorophenyl)amino]-5-fluoropyridin-3-yl}carbonyl)amino]piperidine-1-carboxylate;
tert-butyl 4-[1-(3,4-difluorophenyl)-6-fluoro-2,4-dioxo-1,4-dihydropyrido[2,3-d]pyrimidin-3(2H)-yl]piperidine-1-carboxylate;
tert-butyl (3R)-3-[({2-[(3,4-difluorophenyl)amino]-5-fluoropyridin-3-yl}carbonyl)amino]pyrrolidine-1-carboxylate;
tert-butyl (3R)-3-[1-(3,4-difluorophenyl)-6-fluoro-2,4-dioxo-1,4-dihydropyrido[2,3-d]pyrimidin-3(2H)-yl]pyrrolidine-1-carboxylate;
1-(3,4-difluorophenyl)-6-fluoro-3-[1-(1H-indazol-3-ylcarbonyl)piperidin-4-yl]pyrido[2,3-d]pyrimidine-2,4(1H,3H)-dione;
1-(3,4-difluorophenyl)-6-fluoro-3-{1-[(2R)-2-hydroxy-2-phenylacetyl]piperidin-4-yl}pyrido[2,3-d]pyrimidine-2,4(1H,3H)-dione;
1-(3,4-difluorophenyl)-6-fluoro-3-{1-[2-hydroxy-5-(hydroxymethyl)benzoyl]piperidin-4-yl}pyrido[2,3-d]pyrimidine-2,4(1H,3H)-dione;
1-(3,4-difluorophenyl)-6-fluoro-3-[(3R)-1-(1H-indazol-3-ylcarbonyl)pyrrolidin-3-yl]pyrido[2,3-d]pyrimidine-2,4(1H,3H)-dione;
1-(3,4-difluorophenyl)-6-fluoro-3-{(3R)-1-[(2R)-2-hydroxy-2-phenylacetyl]pyrrolidin-3-yl}pyrido[2,3-d]pyrimidine-2,4(1H,3H)-dione;
3-{(3R)-1-[(2R)-2-cyclohexyl-2-hydroxyacetyl]pyrrolidin-3-yl}-(3,4-difluorophenyl)-6-fluoropyrido[2,3-d]pyrimidine-2,4(1H,3H)-dione;
1-(3,4-difluorophenyl)-6-fluoro-3-{(3R)-1-[2-hydroxy-5-(hydroxymethyl)benzoyl]pyrrolidin-3-yl}pyrido[2,3-d]pyrimidine-2,4(1H,3H)-dione;
1-(3,4-difluorophenyl)-6-fluoro-3-[(3S)-1-(1H-indazol-3-ylcarbonyl)pyrrolidin-3-yl]pyrido[2,3-d]pyrimidine-2,4(1H,3H)-dione;
1-(3,4-difluorophenyl)-6-fluoro-3-{(3S)-1-[(2R)-2-hydroxy-2-phenylacetyl]pyrrolidin-3-yl}pyrido[2,3-d]pyrimidine-2,4(1H,3H)-dione;
N-{cis-4-[1-(3,4-difluorophenyl)-6-fluoro-2,4-dioxo-1,4-dihydropyrido[2,3-d]pyrimidin-3(2H)-yl]cyclohexyl}-1H-indole-5-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-nitrobenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-indole-2-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-benzimidazole-5-carboxamide;
4-cyano-N-{cis-4-[1-(3,4-difluorophenyl)-6-fluoro-2,4-dioxo-1,4-dihydropyrido[2,3-d]pyrimidin-3(2H)-yl]cyclohexyl}benzamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-1,2,3-benzotriazole-5-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1,3-benzothiazole-6-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-methyl-6,7-dihydro-5H-[1,3]thiazolo[3,2-a]pyrimidine-2-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1,2,3-thiadiazole-4-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-pyrazole-4-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}thiophene-3-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-pyrrole-3-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1-methyl-1H-imidazole-4-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-pyrazole-3-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1-methylpiperidine-3-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-morpholin-4-ylpropanamide;
N-{cis-4-[1-(3,4-difluorophenyl)-6-fluoro-2,4-dioxo-1,4-dihydropyrido[2,3-d]pyrimidin-3(2H)-yl]cyclohexyl}-4-vinylbenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-4-ethynylbenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-pyrrolo[2,3-b]pyridine-2-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1,3-benzothiazole-2-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}thieno[2,3-b]pyridine-2-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}thieno[2,3-b]pyrazine-6-carboxamide; or,
N-{cis-4-[1-(3,4-difluorophenyl)-6-fluoro-2,4-dioxo-1,4-dihydropyrido[2,3-d]pyrimidin-3(2H)-yl]cyclohexyl}[1,2,4]triazolo[1,5-a]pyrimidine-2-carboxamide;
   or a pharmaceutically acceptable salt thereof.

The compounds of the present invention can be prepared as described below or by adapting methods known in the art.

In a further aspect the invention provides a process for the preparation of a compound of formula (I) wherein X is C, which comprises removing the Boc protecting group from a compound of formula (II):

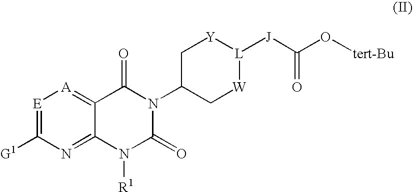

wherein R¹, G¹, E, A, Y, L, W, and J are as defined in formula (I), (for example with an acid such as trifluoroacetic acid or hydrochloric acid) and reacting the product so formed with an acid or acid derivative of formula (III):

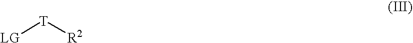

wherein R² and T are as defined in formula (I), and LG is a leaving group. The process is carried out at a suitable temperature, generally between 0° C. and the boiling point of the solvent, in a suitable solvent such as dichloromethane or N-methylpyrrolidinone. The process is optionally carried out in the presence of a base and/or a coupling reagent such as HATU, HOAT, HOBT or DIEA. Suitable leaving groups LG include OH and halogen, particularly OH.

A compound of formula (II) wherein R¹, G¹, E, A, Y, L, W, and J are as defined in formula (I), can be prepared by condensing a compound of formula (IV):

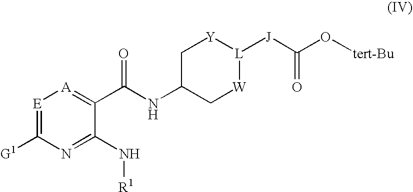

wherein R¹, G¹, E, A, Y, L, W, and J are as defined in formula (I), with a suitable carbonylating agent such as carbonyl diimidazole or ethyl chloroformate in the presence of a suitable base such as sodium hydride. The process is carried out at a suitable temperature, generally between 0° C. and the boiling point of the solvent, in a suitable solvent such as tetrahydrofuran.

In a further aspect the invention provides a process for the preparation of a compound of formula (I) wherein X is S, by reacting a compound of formula (IIa):

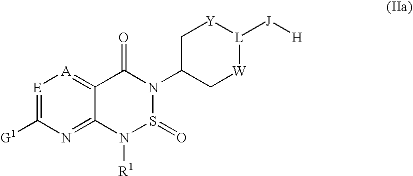

wherein R¹, G¹, E, A, Y, L, W, and J are as defined in formula (I), with an acid or acid derivative of formula (II):

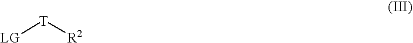

wherein R² and T are as defined in formula (I), and LG is a leaving group. The process is carried out at a suitable temperature, generally between 0° C. and the boiling point of the solvent, in a suitable solvent such as dichloromethane or N-methylpyrrolidinone. The process is optionally carried out in the presence of a base and/or a coupling reagent such as HATU, HOAT, HOBT or DIEA. Suitable leaving groups LG include OH and halogen, particularly OH.

A compound of formula (IIa) wherein R¹, G¹, E, A, Y, L, W, and J are as defined in formula (I), can be prepared by condensing a compound of formula (IV), wherein R¹, G¹, E, A, Y, L, W, and J are as defined in formula (I), with a suitable sulfonylating agent such as thionyl chloride in the presence of a suitable base such as sodium hydride. The process is carried out at a suitable temperature, generally between 0° C. and the boiling point of the solvent, in a suitable solvent such as tetrahydrofuran.

A compound of formula (IV) wherein R¹, G¹, E, A, Y, L, W and J are as defined in formula (I), can be prepared by reacting a compound of formula (V):

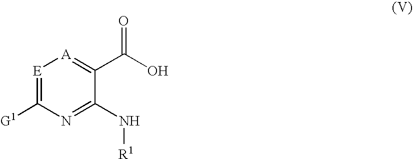

wherein R¹, G¹, E, and A are as defined in formula (I), with an amine of formula (VI)

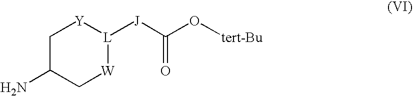

wherein Y, L, W, and J are as defined in formula (I). The process is carried out at a suitable temperature, generally between 0° C. and the boiling point of the solvent, in a suitable solvent such as dichloromethane. The process is optionally carried out in the presence of a base and a coupling reagent such as HATU, HOAT, HOBT or DIEA.

A compound of formula (V) wherein R¹, G¹, E, and A are as defined in formula (I), can be prepared by reacting a compound of formula (VII):

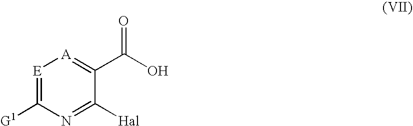

wherein G¹, E, and A are as defined in formula (I) and Hal represents a halogen atom, with an aniline (VIII):

R¹—NH₂ (VIII)

wherein R¹ is as defined in formula (I). The process is carried out at a suitable temperature, generally between 50° C. and the boiling point of the solvent, in a suitable solvent such as dimethylformamide. The process is optionally carried out in the presence of a base such as potassium carbonate.

The preparations of various intermediates are described in the literature or can be prepared by routine adaptation of methods described in the literature. In the above processes it may be desirable or necessary to protect an acid group or a hydroxy or other potentially reactive group. Suitable protecting groups and details of processes for adding and removing such groups may be found in “Protective Groups in Organic Synthesis”, 3rd Edition (1999) by Greene and Wuts.

In another aspect the present invention provides processes for the preparation of compounds of formula (I).

The compounds of formula (I) have activity as pharmaceuticals, in particular as modulators of PDE 4 receptor activity, and may be used in the treatment of inflammatory diseases, asthma or COPD.

Examples of disease states that can be treated with a compound of the invention are

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Atzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

According to a further feature of the present invention there is provided a method for treating a PDE 4 mediated disease state in a mammal; such as man, suffering from, or at risk of, said disease state, which comprises administering to a mammal in need of such treatment a therapeutically effective amount of a compound of the formula (I) or a pharmaceutically acceptable salt thereof.

The invention also provides a compound of the formula (I), or a pharmaceutically acceptable salt thereof, for use in therapy.

In another aspect the invention provides the use of a compound of formula (I), or a pharmaceutically acceptable salt thereof, in the manufacture of a medicament for use in therapy (for example modulating PDE 4 enzymatic activity).

The invention further provides the use of a compound of formula (I), or a pharmaceutically acceptable salt thereof, in the manufacture of a medicament for use in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema; in a mammal (for example man).

In a further aspect the invention provides a compound of formula (I), or a pharmaceutically acceptable salt thereof, for use in the treatment of asthma {such as bronchial, allergic, intrinsic, extrinsic or dust asthma, particularly chronic or inveterate asthma (for example late asthma or airways hyper-responsiveness)}; or COPD.

In a still further aspect a compound of formula (I), or a pharmaceutically acceptable salt thereof, is useful in the treatment of COPD.

The present invention also provides the use of a compound of formula (I), or a pharmaceutically acceptable salt thereof, in the manufacture of a medicament for use in the treatment of asthma {such as bronchial, allergic, intrinsic, extrinsic or dust asthma, particularly chronic or inveterate asthma (for example late asthma or airways hyper-responsiveness)}; or COPD.

In order to use a compound of the invention, or a pharmaceutically acceptable salt thereof, for the therapeutic treatment of a mammal, such as man, said ingredient is normally formulated in accordance with standard pharmaceutical practice as a pharmaceutical composition. Therefore in another aspect the present invention provides a pharmaceutical composition which comprises a compound of the formula (I), or a pharmaceutically acceptable salt thereof (active ingredient), and a pharmaceutically acceptable adjuvant, diluent or carrier.

In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99%/w (percent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), inhalation, oral, rectal or parenteral administration. For these purposes the compounds of this invention may be formulated by means known in the art. A suitable pharmaceutical composition of this invention is one suitable for oral administration in unit dosage form, for example a tablet or capsule which contains between 0.1 mg and 1 g of active ingredient.

Each patient may receive, for example, a dose of 0.001 mgkg⁻¹ to 100 mgkg⁻¹, for example in the range of 0.1 mgkg⁻¹ to 20 mgkg⁻¹, of the active ingredient administered, for example, 1 to 4 times per day.

The invention further relates to a combination therapy wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGP-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; for example collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-yls such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agent including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agents, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B. sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK. sub 1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of p38; (xxv) agent modulating the function of Toll-like receptors (TLR), (xxvi) agent modulating the activity of purinergic receptors such as P2X7; (xxvii) inhibitor of transcription factor activation such as NFkB, API, or STATS; or (xxviii) a non-steroidal glucocorticoid receptor (GR-receptor) agonist.

In a further aspect the present invention provides a combination (for example for the treatment of COPD, asthma or allergic rhinitis) of a compound of formula (I) and one or more agents is selected from the list comprising:
a non-steroidal glucocorticoid receptor (GR-receptor) agonist;
a selective β.sub2. adrenoceptor agonist such as metaproterenol, isoproterenol, isoprenaline, albuterol, salbutamol, formoterol, salmeterol, terbutaline, orciprenaline, bitolterol mesylate, pirbuterol or indacaterol;
a muscarinic receptor antagonist (for example a M1, M2 or M3 antagonist, such as a selective M3 antagonist) such as ipratropium bromide, tiotropium bromide, oxitropium bromide, pirenzepine or telenzepine;
a modulator of chemokine receptor function (such as a CCR1 receptor antagonist); or,
an inhibitor of p38 kinase function.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erb b2 antibody trastuzumab, or the anti-erb b1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/36035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or,
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be illustrated by the following non-limiting examples in which, unless stated otherwise:

(i) when given, ¹H NMR data is quoted and is in the form of delta values for major diagnostic protons, given in parts per million (ppm) relative to tetramethylsilane (TMS) as an internal standard, determined at 300 MHz or 400 MHz using perdeuterio DMSO-d₆ (CD₃SOCD₃) or CDCl₃ as the solvent unless otherwise stated;
(ii) mass spectra (MS) were run with an electron energy of 70 electron volts in the chemical ionisation (CI) mode using a direct exposure probe; where indicated ionisation was effected by electron impact (EI) or fast atom bombardment (FAB); where values for m/z are given, generally only ions which indicate the parent mass are reported, and unless otherwise stated the mass ion quoted is the positive mass ion −(M+H)⁺;
(iii) the title and sub-title compounds of the examples and methods were named using the index name program from Advanced Chemistry Development Inc, version 6.00;
(iv) unless stated otherwise, the following method was used for LC/MS analysis: Instrument Agilent 1100; Column Waters Symmetry 2.1×30 mm; Mass APCI; Flow rate 0.7 mL/min; Wavelength 254 nm; Solvent A: water+0.1% TFA; Solvent B: acetonitrile+0.1% TFA; Gradient 15-95%/B 2.7 min, 95% B 0.3 min;
(v) unless stated otherwise, the following method was used for LC analysis:
Method A: Instrument Agilent 1100; Column: Kromasil C18 100×3 mm, 5 μL particle size, Solvent A: 0.1% TFA/water, Solvent B: 0.08% TFA/acetonitrile Flow: 1 mL/min, Gradient 10-100%/B 20 min, 100% B1 min. Absorption was measured at 220, 254 and 280 nm.
Method B: Instrument Agilent 1100; Column: XTerra C 8, 100×3 mm, 5μ particle size, Solvent A: 15 mM NH₃/water, Solvent B: acetonitrile Flow: 1 mL/min, Gradient 10-100%/B 20 min, 100% B1 min. Absorption was measured at 220, 254 and 280 nm;
(vi) the following abbreviations are used:

| | |
|---|---|
| DMF | N,N-Dimethylformamide |
| NMP | 1-N-Methyl-2-pyrrolidinone |
| HOAT | 1-Hydroxy-7-azabenzotriazole |
| DIEA | N,N-Diisopropylethylamine |
| HATU | O-(7-Benzotriazol-1-yl)-N,N,N′,N′-tetramethyluronium |
| | hexafluorophosphate |
| THF | Tetrahydrofuran |
| DCM | Dichloromethane |
| HPLC | High pressure liquid chromatography |
| d | Day(s) |
| h | Hour(s) |
| min | Minute(s) |

The starting materials for the Examples below are either commercially available or readily prepared by standard methods from known starting materials. For example, the following reactions are an illustration of the preparation of some of the starting materials.

### PREPARATION 1

### 2-[(3,4-Difluorophenyl)amino]-5-fluoronicotinic acid

2-Chloro-5-fluoronicotinic acid (5.0 g, 28.5 mmol), K₂CO₃ (4.7 g, 34.2 mmol) was added to dry DMF (10 ml) in argon atmosphere. Copper (0.11 g, 1.71 mmol), methanol washed and dried copper (I) bromide (0.16 g, 1.14 mmol) and 3,4-difluoroaniline (4.8 ml, 48.4 mmol) were added and the mixture was stirred in 150° C. oil bath. Product was formed after 1.5 h according to LC-MS. The mixture was poured into 1 M HCl whereupon the precipitate was filtered, washed with 1 M HCl, dissolved in saturated Na₂CO₃ solution and extracted with ethyl acetate. The organic phase was dried with Mg₂SO₄ and evaporated to obtain the title compound (5.3 g, 69%).

APCI-MS m/z: 269 [MH⁺].

### PREPARATION 2

### tert-Butyl {cis-4-[({2-[(3,4-difluorophenyl)amino]-5-fluoropyridin-3-yl}carbonyl)amino]cyclohexyl}carbamate

2-[(3,4-Difluorophenyl)amino]-5-fluoronicotinic acid (0.56 g, 2.09 mmol), N-(3-dimethylaminopropyl)-N′-ethylcarbodiimide hydrochloride (0.60 g, 3.14 mmol), 1-hydroxy-7-azabenzotriazole (0.43 g, 3.14 mmol) and N,N-diisopropylethylamine (1.07 ml, 6.27 mmol) were stirred in CH₂Cl₂. To the mixture tert-butyl (cis-4-aminocyclohexyl)carbamate (0.49 g, 2.30 mmol) was added. Product was formed according to LC-MS after 1 h. Ethyl acetate was added and the mixture was washed with water. The organic solvent was evaporated. The crude product was purified on a silica plug and was eluted with ethyl acetate/heptane (1:1) to give the title compound (0.90 g, 93

APCI-MS m/z: 465.2 [MH⁺].

### PREPARATION 3

### tert-Butyl {cis-4-[1-(3,4-difluorophenyl)-6-fluoro-2,4-dioxo-1,4-dihydropyrido[2,3-d]pyrimidin-3(2H)-yl]cyclohexyl}carbamate

Sodium hydride (55% in oil; 0.80 g, 18.21 mmol) was added to a solution of tert-butyl {cis-4-[({2-[(3,4-difluorophenyl)amino]-5-fluoropyridin-3-yl}carbonyl)amino]-cyclohexyl}carbamate (2.82 g, 6.07 mmol) in dry THF (10 ml) under an argon atmosphere. The mixture was stirred for 30 min and then cooled on ice. Ethyl chloroformate (2.92 ml, 30.36 mmol) was added and the mixture was heated in 80° C. oil bath for 40 min. NH₄Cl-solution was added and the mixture was extracted with ethyl acetate followed by evaporation of the organic layer. The crude product was purified by flash chromatography [ethyl acetate/heptane (1:4)] followed by HPLC (MeCN 70%-95%) to obtain the title compound (1.87 g, 63%).

APCI-MS m/z: 391.1 [MH⁺].

¹H NMR (300 MHz, DMSO-d₆) δ 8.59 (d, 1H), 8.30 (m, 1H), 7.65-7.55 (m, 2H), 7.33-7.27 (m, 1H), 6.54 (s, 1H), 4.73 (t, 1H), 3.55 (s, 1H), 2.57 (d, 2H), 1.90 (d, 2H), 1.54-1.43 (m, 4H), 1.39 (s, 9H).

### PREPARATION 4

### 3-(cis-4-Aminocyclohexyl)-1-(3,4-difluorophenyl)-6-fluoro-1H-pyrido[2,3-c][1,2,6]thiadiazin-4(3H)-one 2-oxide

tert-Butyl {cis-4-[({2-[(3,4-difluorophenyl)amino]-5-fluoropyridin-3-yl}carbonyl)amino]cyclohexyl}carbamate (0.18 g, 0.38 mmol; made in accordance to

### PREPARATION 2), was dissolved in dry THF (1 ml) under an argon atmosphere. Sodium hydride (55% in oil; 66 mg, 1.51 mmol) was added to the solution and the mixture was stirred for 0.5 h. The mixture was then cooled on ice and thionyl chloride (138 μl, 1.89 mmol) was added followed by reflux at 80° C. for 1 h. When the product was formed according to LC-MS a solution of Na₂CO₃ was added and the mixture was extracted with ethyl acetate. Purification on preparative HPLC gave the title compound (25 mg, 16%) which reversed to the starting material in aqueous conditions.

APCI-MS m/z: 411 [MH⁺].

### PREPARATION 5

### 5-Fluoro-2-{[3-(methylthio)phenyl]amino}nicotinic acid

2-Chloro-5-fluoronicotinic acid (2.11 g, 12 mmol), K₂CO₃ (1.99 g, 14.4 mmol) was added to dry NMP (8 ml) under argon atmosphere. Copper (46 mg, 0.72 mmol), methanol-washed dried copper (I) bromide (86 mg, 0.6 mmol) and 3-(methylthio)aniline (2.51 ml, 20.4 mmol) were added and the mixture was stirred in 150° C. oil bath. Product was formed after 2 h according to LC-MS. The mixture was poured into 1 M HCl (20 ml) whereupon a precipitate formed and was filtered, washed with 1 M HCl, water and dried in vacuum at 50° C. to afford the title compound (1.72 g, 52%).

APCI-MS m/z: 279 [MH⁺].

### PREPARATION 6

### tert-Butyl (cis-4-{[(5-fluoro-2-{[3-(methylthio)phenyl]amino}pyridin-3-yl)carbonyl]amino}cyclohexyl)carbamate

A mixture of 5-fluoro-2-{[3-(methylthio)phenyl]amino}nicotinic acid (1.72 g, 6.2 mmol), tert-butyl (cis-4-aminocyclohexyl)carbamate (1.46 g, 6.8 mmol), HATU (2.81 g, 7.4 mmol), HOAT (1.01 mg, 7.4 mmol) and DIEA (3.45 ml, 20 mmol) in NMP (20 ml) was stirred for 10 min at room temperature (at pH 8-9 adjusted with DIEA). Ethyl acetate was added and the crude product was washed twice with 0.5 M aqueous citric acid, aqueous sodium hydrogencarbonate and water. The organic solvents dried over Na₂SO₄, filtered and removed in vacuum. The residue was purified by flash chromatography on silica using ethyl acetate/heptane (1:2) as eluent to give the title compound (2 g, 68%).

¹H NMR (DMSO-d₆): δ 10.63 (1H, s); 8.46 (1H, d); 8.36 (1H, d); 8.13 (1H, dd); 7.63 (1H, brs); 7.34 (1H, d); 7.22 (1H, t); 6.85 (1H, d); 6.64 (1H, brs); 3.84 (1H, brs); 3.42 (1H, brs); 2.47 (3H, s); 1.73 (4H, m); 1.56 (4H, m); 1.39 (9H, s).

APCI-MS m/z: 475 [MH⁺].

LC (method A) rt=15.1 min

### PREPARATION 7

### tert-Butyl{cis-4-[6-fluoro-1-[3-(methylthio)phenyl]-2,4-dioxo-1,4-dihydropyrido[2,3-d]pyrimidin-3(2H)-yl]coclohexyl}carbamate

To a solution of tert-butyl (cis-4-{[(5-fluoro-2-{[3-(methylthio)phenyl]amino}-pyridin-3-yl)carbonyl]amino}cyclohexyl)carbamate (0.95 g, 2 mmol) in a mixture of dry NMP (6 ml) and dry THF (3 ml) under argon atmosphere 50% NaH in oil (0.30 g, 6 mmol) was added. The mixture was stirred for 30 min and then cooled on ice. Ethyl chloroformate (1.05 ml, 10 mmol) was added and the mixture was heated at 80° C. for 2 h. Ethyl acetate was added and the crude product was washed twice with 0.5 M aqueous citric acid, aqueous sodium hydrogencarbonate, and water. The organic solvents dried over Na₂SO₄, filtered and removed in vacuum. The residue was purified by flash chromatography on silica using ethyl acetate/heptane (1:2) as eluent to give the title compound (0.52 g, 52%).

¹H NMR (DMSO-d₆): δ 8.58 (1H, d); 8.27 (1H, dd); 7.45 (1H, t); 7.34 (1H, d); 7.30 (1H, brs); 7.14 (1H, d); 6.51 (1H, brs); 4.74 (1H, brt); 3.55 (1H, brs); 2.57 (2H, m); 2.47 (3H, s); 1.90 (2H, brd); 1.47 (4H, m); 1.39 (9H, s).

APCI-MS m/z: 401 [MH⁺-tBOC].

LC (method A) rt=13.6 min

### PREPARATION 8

### 5-Fluoro-2-{[3-(methoxycarbonyl)-phenyl]amino}nicotinic acid

2-Chloro-5-fluoronicotinic acid (2.11 g, 12 mmol), K₂CO₃ (1.99 g, 14.4 mmol) was added to dry NMP (8 ml) under argon atmosphere. Copper (46 mg, 0.72 mmol), methanol-washed dried copper (I) bromide (86 mg, 0.6 mmol) and methyl 3-aminobenzoate (3.08 ml, 20.4 mmol) were added and the mixture was stirred in 150° C. oil bath. Product was formed after 2 h according to LC-MS. The mixture was poured into 1 M HCl (20 ml) whereupon a precipitate formed and was filtered, washed with 1 M HCl, water and dried in vacuum at 50° C. to afford the title compound (2 g, 57%).

APCI-MS m/z: 291 [MH⁺].

### PREPARATION 9

### Methyl 3-({3-[({cis-4-[(tert-butoxycarbonyl)amino]cyclohexyl}amino)carbonyl]-5-fluoropyridin-2-yl}amino)benzoate

A mixture of 5-fluoro-2-{[3-(methoxycarbonyl)phenyl]amino}nicotinic acid (2.91 g, 10 mmol), tert-butyl (cis-4-aminocyclohexyl)carbamate (2.57 g, 12 mmol), HATU (4.57 g, 12 mmol), HOAT (1.63 mg, 12 mmol) and DIEA (5.15 ml, 30 mmol) in NMP (30 ml) was stirred for 10 min at room temperature (at pH 8-9 adjusted with DIEA). Ethyl acetate was added and the crude product was washed twice with 0.5 M aqueous citric acid, aqueous sodium hydrogencarbonate, and water. The organic solvents dried over Na₂SO₄, filtered and removed in vacuum. The residue was purified by flash chromatography on silica using ethyl acetate/heptane (1:2) as eluent to give the title compound (2.85 g, 59%).

¹H NMR (DMSO-d₆): δ 10.79 (1H, s); 8.50 (1H, d); 8.38 (1H, d); 8.25 (1H, brs); 8.16 (1H, dd); 7.88 (1H, d); 7.55 (1H, d); 7.43 (1H, t); 6.65 (1H, brs); 3.86 (4H, brs); 3.42 (1H, brs); 1.74 (4H, m); 1.56 (4H, m); 1.39 (9H, s).

APCI-MS m/z: 487 [MH⁺].

LC (method A) rt=14.0 min

### PREPARATION 10

### Methyl 3-[3-{cis-4-[(tert-butoxycarbonyl)amino]cyclohexyl}-6-fluoro-2,4-dioxo-3,4-dihydropyrido[2,3-d]pyrimidin-1(2H)-yl]benzoate

To a solution of methyl 3-({3-[({cis-4-[(tert-butoxycarbonyl)amino]cyclohexyl}-amino)carbonyl]-5-fluoropyridin-2-yl}amino)benzoate (50 mg, 0.1 mmol) in dry NMP (2 ml) under argon atmosphere 50% NaH in oil (14 mg, 0.3 mmol) and 1,1′-carbonyldiimidazole (50 mg, 0.3 mmol) were added. The mixture was stirred at room temperature for one hour. Ethyl acetate was added and the crude product was washed twice with 0.5 M aqueous citric acid, aqueous sodium hydrogencarbonate, and water. The organic solvents dried over Na₂SO₄, filtered and removed in vacuum. Purification on preparative HPLC gave the title compound (40 mg, 78%).

¹H NMR (DMSO-d₆): δ 8.55 (1H, d); 8.28 (1H, dd); 8.04 (1H, t); 8.01 (1H, brs); 7.68 (2H, brd); 4.74 (1H, brt); 3.87 (3H, s); 3.54 (1H, brs); 2.57 (2H, m); 1.90 (2H, brd); 1.49 (4H, m); 1.39 (9H, s).

APCI-MS m/z: 413 [MH⁺-tBOC].

LC (method A) rt=12.9 min

### PREPARATION 11

### 5-Fluoro-2-{[3-(methylsulfonyl)phenyl]amino}nicotinic acid

### 2-Chloro-5-fluoronicotinic acid (1.06, 6 mmol), K₂CO₃ (2.4 g, 17.4 mmol) was added to dry NMP (5 ml) under argon atmosphere. Copper (23 mg, 0.36 mmol), methanol-washed dried copper (I) bromide (43 mg, 0.3 mmol) and 3-(methylsulfonyl)aniline hydrochloride (2.12 ml, 10.2 mmol) were added and the mixture was stirred in 150° C. oil bath. Product was formed after 2 h according to LC-MS. The mixture was poured into 1 M HCl (20 ml) whereupon a precipitate formed and was filtered, washed with 1 M HCl, water and dried in vacuum at 50° C. to afford the title compound (0.6 g, 32%).

APCI-MS m/z: 311 [MH⁺].

### PREPARATION 12

### tert-Butyl (cis-4-{[(5-fluoro-2-{[3-(methylsulfonyl phenyl]amino}pyridin-3-yl)carbonyl]amino}cyclohexyl)carbamate

A mixture of 5-fluoro-2-{[3-(methylsulfonyl)phenyl]amino}nicotinic acid (0.56 g, 1.8 mmol), tert-butyl (cis-4-aminocyclohexyl)carbamate (0.43 g, 2 mmol), HATU (0.823 g, 2.17 mmol), HOAT (0.3 mg, 2.17 mmol) and DIEA (0.93 ml, 5.4 mmol) in NMP (7 ml) was stirred for 10 min at room temperature (at pH 8-9 adjusted with DIEA). Ethyl acetate was added and the crude product was washed twice with 0.5 M aqueous citric acid, aqueous sodium hydrogencarbonate, and water. The organic solvents dried over Na₂SO₄, filtered and removed in vacuum. The residue was purified by flash chromatography on silica using ethyl acetate/heptane (1:2) as eluent to give the title compound (0.38 g, 42%).

¹H NMR (DMSO-d₆): δ 10.91 (1H, s); 8.52 (1H, d); 8.41 (1H, d); 8.26 (1H, brs); 8.18 (1H, dd); 7.93 (1H, d); 7.55 (1H, t); 7.48 (1H, d); 6.65 (1H, brs); 3.86 (1H, brs); 3.43 (1H, brs); 3.20 (3H, s); 1.74 (4H, m); 1.56 (4H, m); 1.39 (9H, s).

APCI-MS m/z: 507 [MH⁺].

LC (method A) rt=11.8 min

### PREPARATION 13

### tert-Butyl {cis-4-[6-fluoro-1-[3-(methylsulfonyl)phenyl]-2,4-dioxo-1,4-dihydropyrido[2,3-d]pyrimidin-3(2H)-yl]cyclohexyl}carbamate

To a solution of tert-butyl (cis-4-{[(5-fluoro-2-{[3-(methylsulfonyl)phenyl]amino}-pyridin-3-yl)carbonyl]amino}cyclohexyl)carbamate (350 mg, 0.69 mmol) in dry NMP (10 ml) under argon atmosphere 50% NaH in oil (99 mg, 2.07 mmol) and 1,1′-carbonyldiimidazole (336 mg, 2.07 mmol) were added. The mixture was stirred at roomtemperature for one hour. Ethyl acetate was added and the crude product was washed twice with 0.5 M aqueous citric acid, aqueous sodium hydrogencarbonate, and water. The organic solvents dried over Na₂SO₄, filtered and removed in vacuum. Purification on preparative HPLC gave the title compound (180 mg, 49%).

¹H NMR (DMSO-d₆): δ 8.57 (1H, d); 8.30 (1H, dd); 8.04 (1H, d); 8.02 (1H, brs); 7.82 (1H, t); 7.77 (1H, d); 4.74 (1H, brt); 3.55 (1H, brs); 3.28 (3H, s); 2.58 (2H, brd); 1.91 (2H, brd); 1.49 (4H, m); 1.39 (9H, s).

APCI-MS m/z: 433 [MH⁺-tBOC].

LC (method A) rt=11.2 min

### PREPARATION 14

### a) tert-butyl 4-[({2-[(3,4-difluorophenyl)amino]-5-fluoropyridin-3-yl}carbonyl)amino]piperidine-1-carboxylate

A mixture of 2-[(3,4-difluorophenyl)amino]-5-fluoronicotinic acid (600 mg, 2.3 mmol), tert-butyl 4-aminopiperidine-1-carboxylate (501 mg, 2.5 mmol), HATU (951 mg, 2.5 mmol), HOAT (340 mg, 2.5 mmol) and DIEA (1.17 ml, 6.8 mmol) in CH₂Cl₂ (4 ml) was stirred for 2 h at room temperature. CH₂Cl₂ was added and the crude product was washed with aqueous sodium hydrogencarbonate and water. The organic phase was dried over Na₂SO₄, filtered and removed in vacuum. The residue was purified on a silica plug using ethyl acetate/heptane (1:1) as eluent to give the title compound (660 mg, 64%).

APCI-MS m/z: 451.5 [MH⁺].

### b) tert-butyl 4-[1-(3,4-difluorophenyl)-6-fluoro-2,4-dioxo-1,4-dihydropyrido[2,3-d]pyrimidin-3 (2H)-yl]piperidine-1-carboxylate

To a solution of tert-butyl 4-[({2-[(3,4-difluorophenyl)amino]-5-fluoropyridin-3-yl}carbonyl)amino]piperidine-1-carboxylate (660 mg, 1.47 mmol) and 1,1′-carbonyldiimidazole (713 mg, 4.40 mmol) in dry NMP (5 ml) under argon atmosphere 50% NaH in oil (211 mg, 4.40 mmol) was added. The mixture was stirred at room temperature over night. Water was added and the crude product was extracted with ethyl acetate. The organic solvents were dried over Na₂SO₄, filtered and removed in vacuum. The residue was purified on silica plug using ethyl acetate/heptane (1:2) as eluent to give the title compound 90% pure (524 mg, 75%).

APCI-MS m/z: 377 [MH⁺].

### c) 1-(3,4-difluorophenyl)-6-fluoro-3-piperidin-4-ylpyrido[2,3-d]pyrimidine-2,4(1H,3H)-dione hydrochloride

A mixture of tert-butyl 4-[1-(3,4-difluorophenyl)-6-fluoro-2,4-dioxo-1,4-dihydropyrido[2,3-d]pyrimidin-3(2H)-yl]piperidine-1-carboxylate (0.05 g, 0.104 mmol) and 4 M HCl in 1,4-Dioxane (2 ml) was stirred at room temperature for one hour. The solvents were removed and the pure crude product was used directly.

APCI-MS m/z: 377.1 [MH⁺].

### PREPARATION 15

### a) tert-butyl (3R)-3-[({2-[(3,4-difluorophenyl)amino]-5-fluoropyridin-3-yl}carbonyl)amino]pyrrolidine-1-carboxylate

A mixture of 2-[(3,4-difluorophenyl)amino]-5-fluoronicotinic acid (600 mg, 2.3 mmol), tert-butyl (3R)-3-aminopyrrolidine-1-carboxylate (425 μL, 2.5 mmol), HATU (951 mg, 2.5 mmol), HOAT (340 mg, 2.5 mmol) and DIEA (1.17 ml, 6.8 mmol) in CH₂Cl₂ (4 ml) was stirred for 2 h at room temperature. CH₂Cl₂ was added and the crude product was washed with aqueous sodium hydrogencarbonate and water. The organic phase was dried over Na₂SO₄, filtered and removed in vacuum. The residue was purified on a silica plug using ethyl acetate/heptane (1:1) as eluent to give the title compound (613 mg, 62%).

APCI-MS m/z: 437.5 [MH⁺].

### b) tert-butyl (3R)-3-[1-(3,4-difluorophenyl)-6-fluoro-2,4-dioxo-1,4-dihydropyrido[2,3-d]pyrimidin-3 (2H)-yl]pyrrolidine-1-carboxylate

To a solution of tert-butyl (3R)-3-[({2-[(3,4-difluorophenyl)amino]-5-fluoropyridin-3-yl}carbonyl)amino]pyrrolidine-1-carboxylate (613 mg, 1.40 mmol) and 1,1′-carbonyldiimidazole (683 mg, 4.21 mmol) in dry NMP (3 ml) under argon atmosphere 50% NaH in oil (202 mg, 4.21 mmol) was added. The mixture was stirred at room temperature over night. Water was added and the crude product was extracted with ethyl acetate. The organic solvents were dried over Na₂SO₄, filtered and removed in vacuum. The residue was purified through a silica plug using ethyl acetate/heptane (1:2) as eluent to give the title compound (294 mg, 45%).

APCI-MS m/z: 363.2 [MH⁺].

### c) 1-(3,4-difluorophenyl)-6-fluoro-3-[(3R)-pyrrolidin-3-yl]pyrido[2,3-d]pyrimidine-2,4(1H,3H)-dione hydrochloride

A mixture of tert-butyl (3R)-3-[1-(3,4-difluorophenyl)-6-fluoro-2,4-dioxo-1,4-dihydropyrido[2,3-d]pyrimidin-3(2H)-yl]pyrrolidine-1-carboxylate (0.053 g, 0.114 mmol) and 4 M HCl in 1,4-Dioxane (2 ml) was stirred at room temperature for one hour. The solvents were removed and the pure crude product was used directly.

APCI-MS m/z: 363.1 [MH⁺].

### PREPARATION 16

### a) tert-butyl (3S)-3-[({2-[(3,4-difluorophenyl)amino]-5-fluoropyridin-3-yl}carbonyl)amino]pyrrolidine-1-carboxylate

A mixture of 2-[(3,4-difluorophenyl)amino]-5-fluoronicotinic acid (600 mg, 2.3 mmol), tert-butyl (3S)-3-aminopyrrolidine-1-carboxylate (425 μl, 2.5 mmol), HATU (951 mg, 2.5 mmol), HOAT (340 mg, 2.5 mmol) and DIEA (1.17 ml, 6.8 mmol) in CH₂Cl₂ (4 ml) was stirred for 2 h at room temperature. CH₂Cl₂ was added and the crude product was washed with aqueous sodium hydrogencarbonate and water. The organic phase was dried over Na₂SO₄, filtered and removed in vacuum. The residue was purified on a silica plug using ethyl acetate/heptane (1:1) as eluent to give the title compound (530 mg, 53%).

APCI-MS m/z: 437.1 [MH⁺].

### b) tert-butyl (3S)-3-[1-(3,4-difluorophenyl)-6-fluoro-2,4-dioxo-1,4-dihydropyrido[2,3-d]pyrimidin-3(2H)-yl]pyrrolidine-1-carboxylate

To a solution of tert-butyl (3S)-3-[({2-[(3,4-difluorophenyl)amino]-5-fluoropyridin-3-yl}carbonyl)amino]pyrrolidine-1-carboxylate (530 mg, 1.21 mmol) and 1,1′-carbonyldiimidazole (590 mg, 3.64 mmol) in dry NMP (3 ml) under argon atmosphere 50% NaH in oil (175 mg, 3.64 mmol) was added. The mixture was stirred at room temperature over night. Water was added and the crude product was extracted with ethyl acetate. The organic phase was dried over Na₂SO₄, filtered and removed in vacuum The residue was purified through a silica plug using ethyl acetate/heptane (1:3) as eluent to give the title compound as an oil 85% pure (500 mg, 89%).

APCI-MS m/z: 363 [MH⁺].

### c) 1-(3,4-difluorophenyl)-6-fluoro-3-[(3S)-pyrrolidin-3-yl]pyrido[2,3-d]pyrimidine-2,4(1H,3H)-dione hydrochloride

A mixture of tert-butyl (3S)-3-[1-(3,4-difluorophenyl)-6-fluoro-2,4-dioxo-1,4-dihydropyrido[2,3-d]pyrimidin-3(2H)-yl]pyrrolidine-1-carboxylate (0.08 g, 0.17 mmol) and 4 M HCl in 1,4-Dioxane (2 ml) was stirred at room temperature for one hour. The solvents were removed and the pure crude product was used directly.

APCI-MS m/z: 363 [MH⁺].

### PREPARATION 17

### 3-(cis-4-Aminocyclohexyl)-1-(3,4-difluorophenyl)-6-fluoro-1H-pyrido[2,3-c][1,2,6]thiadiazin-4(3H)-one 2-oxide

tert-Butyl {cis-4-[({2-[(3,4-difluorophenyl)amino]-5-fluoropyridin-3-yl}carbonyl)amino]cyclohexyl}carbamate (175 mg, 0.38 mmol) was dissolved in dry THF (1 ml) in argon atmosphere. 55% NaH in oil (66 mg, 1.5 mmol) was added and the mixture was stirred for 0.5 h. The mixture was cooled on ice and thionyl chloride (138 μl, 1.9 mmol) added followed by reflux at 80° C. for 1 h. At completion of the reaction according to LC-MS, Na₂CO₃ (aq) was added and the mixture was extracted with ethyl acetate. Purification on preparative HPLC provided the title compound (25 mg, 16%).

APCI-MS m/z: 411 [MH⁺].

### EXAMPLE 1

This Example illustrates the preparation of N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-5-(hydroxymethyl)benzamide.

### Step a: 3-(cis-4-Aminocyclohexyl)-1-(3,4-difluorophenyl)-6-fluoropyrido[2,3-d]pyrimidine-2,4(1H,3H)-dione hydrochloride

A mixture of tert-butyl {cis-4-[1-(3,4-difluorophenyl)-6-fluoro-2,4-dioxo-1,4-dihydropyrido[2,3-d]pyrimidin-3(2H)-yl]cyclohexyl}carbamate (98 mg, 0.2 mmol) and 4 M HCl in 1,4-dioxane (2 mL) was stirred at room temperature for one hour. The solvents were removed and the pure crude product was used directly in the next step.

APCI-MS m/z: 391 [MH⁺].

### Step b: N-{cis-4-[1-(3,4-Difluorophenyl)-6-fluoro-2,4-dioxo-1,4-dihydropyrido[2,3-d]pyrimidin-3(2H)-yl]cyclohexyl}-2-hydroxy-5-(hydroxymethyl)benzamide

A mixture of 3-(cis-4-aminocyclohexyl)-1-(3,4-difluorophenyl)-6-fluoropyrido[2,3-d]pyrimidine-2,4(1H,3H)-dione hydrochloride (85 mg, 0.2 mmol), 2-hydroxy-5-(hydroxymethyl)benzoic acid (45 mg, 0.26 mmol), HATU (100 mg, 0.26 mmol), HOAT (36 mg, 0.26 mmol) and DIEA (200 μL, 1.16 mmol) in NMP (3.5 mL) was stirred for 10 min at room temperature (at pH 8-9). Ethyl acetate was added and the solution of crude product was washed twice with aqueous sodium hydrogencarbonate, 0.5 M aqueous citric acid and water. The organic solution was dried over Na₂SO₄, filtered and solvent removed in vacuum. The residue was purified by preparative HPLC to give the title compound (46 mg, 43%).

¹H NMR (400 MHz, DMSO-d₆): δ 11.66 (1H, s); 8.61 (1H, s); 8.59 (1H, d); 8.31 (1H, dd); 7.92 (1H, d); 7.65-7.56 (2H, m); 7.31 (2H, m); 6.91 (1H, d); 4.84 (1H, t); 4.40 (2H, s); 4.16 (1H, m); 3.36 (1H, m); 2.69-2.53 (2H, m) 2.00; (2H, m); 1.74-1.58 (4H, m).

APCI-MS m/z: 541 [MH⁺].

LC (method A) rt=10.5 min

### EXAMPLES 2 to 47

The following compounds were synthesised following the procedure described in Example 1.

| Ex | Compound | ¹H NMR | m/z |
|---|---|---|---|
| 2 | N-{cis-4-[1-(3,4- | (400 MHz, DMSO-d₆) δ 8.60 (d, 1H), | 525.2 |
| | difluorophenyl)-6-fluoro-2,4- | 8.35 (dd, 1H), 7.66-7.59 (m, 3H), | |
| | dioxo-1,4-dihydropyrido[2,3- | 7.44 (d, 2H), 7.34-7.23 (m, 4H), 4.96 (s, 1H), | |
| | d]pyrimidin-3(2H)- | 4.80 (t, 1H), 3.91 (s, 1H), 2.57 (d, 1H), | |
| | yl]cyclohexyl}-2-hydroxy-2- | *(1H in solvent peak), 1.85-1.77 (m, | |
| | phenylacetamide | 2H), 1.65-1.55 (m, 4H) | |
| 3 | N-{cis-4-[1-(3,4- | (400 MHz, DMSO-d₆) δ 8.60 (d, 1H), | 449.2 |
| | difluorophenyl)-6-fluoro-2,4- | 8.33 (dd, 1H), 7.64-7.57 (m, 2H), | |
| | dioxo-1,4-dihydropyrido[2,3- | 7.31-7.29 (m, 1H), 4.80 (t, 1H), 3.99 (s, 1H), | |
| | d]pyrimidin-3(2H)- | 3.81 (s, 2H), *(2H in solvent peak), | |
| | yl]cyclohexyl}-2- | 1.81 (d, 2H), 1.66-1.56 (m, 4H) | |
| | hydroxyacetamide | | |
| 4 | 5-chloro-N-{cis-4-[1-(3,4- | (400 MHz, DMSO-d₆) δ 8.63-8.59 (m, | 545.2, |
| | difluorophenyl)-6-fluoro-2,4- | 2H), 8.31 (dd, 1H), 7.98 (d, 1H), | 547.1 |
| | dioxo-1,4-dihydropyrido[2,3- | 7.64-7.57 (m, 2H), 7.42 (dd, 1H), | |
| | d]pyrimidin-3(2H)- | 7.32-7.29 (m, 1H), 7.00 (d, 1H), 4.84 (t, 1H), | |
| | yl]cyclohexyl}-2- | 4.15 (s, 1H), 2.67-2.55 (m, 2H), 2.00 (d, 2H), | |
| | hydroxybenzamide | 1.72-1.60 (m, 4H) | |
| 5 | N-{cis-4-[1-(3,4- | (500 MHz, DMSO-d₆) δ 9.59 (s, 1H), | 511.1 |
| | difluorophenyl)-6-fluoro-2,4- | 8.56 (d, 1H), 8.29 (dd, 1H), 7.77 (d, | |
| | dioxo-1,4-dihydropyrido[2,3- | 1H), 7.60-7.54 (m, 2H), 7.29-7.18 (m, | |
| | d]pyrimidin-3(2H)- | 4H), 6.87 (dt, 1H), 4.78 (t, 1H), 3.92 (s, | |
| | yl]cyclohexyl}-3- | 1H), 2.71-2.66 (m, 1H), *(1H in solvent | |
| | hydroxybenzamide | peak), 2.06 (d, 2H), 1.60-1.46 (m, 4H) | |
| 6 | N-{cis-4-[1-(3,4- | (500 MHz, DMSO-d₆) δ 9.88 (s, 1H), | 511.1 |
| | difluorophenyl)-6-fluoro-2,4- | 8.56 (d, 1H), 8.30 (dd, 1H), 7.70 (d, | |
| | dioxo-1,4-dihydropyrido[2,3- | 2H), 7.60-7.54 (m, 3H), 7.29-7.26 (m, | |
| | d]pyrimidin-3(2H)- | 1H), 6.76 (d, 2H), 4.78 (t, 1H), 3.92 (s, | |
| | yl]cyclohexyl}-4- | 1H), 2.72-2.66 (m, 1H), *(1H in solvent | |
| | hydroxybenzamide | peak), 2.05 (d, 2H), 1.60-1.45 (m, 4H) | |
| 7 | methyl 2-[({cis-4-[1-(3,4- | (500 MHz, DMSO-d₆) δ 8.55 (d, 1H), | 553.1, |
| | difluorophenyl)-6-fluoro-2,4- | 8.26 (dd, 1H), 8.22 (d, 1H), 7.73 (dd, | 554.2 |
| | dioxo-1,4-dihydropyrido[2,3- | 1H), 7.60-7.48 (m, 4H), 7.46 (d, 1H), | |
| | d]pyrimidin-3(2H)- | 7.28-7.24 (m, 1H), 4.75 (t, 1H), 3.92 (s, | |
| | yl]cyclohexyl}amino)carbonyl]- | 1H), 3.69 (s, 3H), 2.72-2.66 (m, 1H), | |
| | benzoate | *(1H in solvent peak), 2.03 (d, 2H), | |
| | | 1.60-1.45 (m, 4H) | |
| 8 | N-{cis-4-[1-(3,4- | (500 MHz, DMSO-d₆) δ 8.57 (d, 1H), | 525.2, |
| | difluorophenyl)-6-fluoro-2,4- | 8.28 (dd, 1H), 7.78 (d, 1H), | 526.2 |
| | dioxo-1,4-dihydropyrido[2,3- | 7.61-7.55 (m, 2H), 7.31-7.26 (m, 1H), 7.05 (d, | |
| | d]pyrimidin-3(2H)- | 2H), 6.61 (d, 2H), 4.71 (t, 1H), 3.73 (s, | |
| | yl]cyclohexyl}-2-(4- | 1H), *(4H in solvent peak), 1.84 (d, 2H), | |
| | hydroxyphenyl)acetamide | 1.54-1.42 (m, 4H) | |
| 9 | N-{cis-4-[1-(3,4- | (500 MHz, DMSO-d₆) δ 11.83 (s, 1H), | 525.1, |
| | difluorophenyl)-6-fluoro-2,4- | 8.56 (d, 1H), 8.43 (d, 1H), 8.27 (dd, | 526.1 |
| | dioxo-1,4-dihydropyrido[2,3- | 1H), 7.82 (d, 1H), 7.60-7.55 (m, 2H), | |
| | d]pyrimidin-3(2H)- | 7.27 (d, 1H), 6.71-6.68 (m, 2H), 4.79 (t, | |
| | yl]cyclohexyl}-2-hydroxy-4- | 1H), 4.09 (s, 1H), *(2H in solvent peak), | |
| | methylbenzamide | 2.23 (s, 3H), 1.98 (d, 2H), 1.67-1.53 (m, | |
| | | 4H) | |
| 10 | N-{cis-4-[1-(3,4- | (500 MHz, DMSO-d₆) δ 11.81 (s, 1H), | 511.1, |
| | difluorophenyl)-6-fluoro-2,4- | 8.57-8.54 (m, 2H), 8.27 (dd, 1H), | 513.1 |
| | dioxo-1,4-dihydropyrido[2,3- | 7.92 (dd, 1H), 7.60-7.54 (m, 2H), | |
| | d]pyrimidin-3(2H)- | 7.35-7.31 (m, 1H), 7.27 (d, 1H), 6.92-6.86 (m, | |
| | yl]cyclohexyl}-2- | 2H), 4.80 (t, 1H), 4.11 (s, 1H), *(2H in | |
| | hydroxybenzamide | solvent peak), 1.97 (d, 2H), | |
| | | 1.67-1.55 (m, 4H) | |
| 11 | N-{cis-4-[1-(3,4- | (500 MHz, DMSO-d₆) δ 12.32 (s, 1H), | 512.2 |
| | difluorophenyl)-6-fluoro-2,4- | 8.56 (d, 1H), 8.44 (d, 1H), 8.31 (dd, | |
| | dioxo-1,4-dihydropyrido[2,3- | 1H), 8.17 (dd, 1H), 7.61-7.51 (m, 3H), | |
| | d]pyrimidin-3(2H)- | 7.41 (dd, 1H), 7.29-7.25 (m, 1H), | |
| | yl]cyclohexyl}-3- | 4.82 (t, 1H), 4.15 (s, 1H), *(2H in solvent | |
| | hydroxypyridine-2- | peak), 1.99 (d, 2H), 1.72 (t, 2H), | |
| | carboxamide | 1.62 (d, 2H) | |
| 12 | 3-chloro-N-{cis-4-[1-(3,4- | (500 MHz, DMSO-d₆) δ 10.69 (s, 1H), | 545, |
| | difluorophenyl)-6-fluoro-2,4- | 8.56 (d, 1H), 8.29 (dd, 1H), 7.91 (d, | 547.1 |
| | dioxo-1,4-dihydropyrido[2,3- | 1H), 7.77 (d, 1H), 7.65 (dd, 1H), | |
| | d]pyrimidin-3(2H)- | 7.59-7.54 (m, 2H), 7.29-7.25 (m, 1H), | |
| | yl]cyclohexyl}-4- | 6.96 (d, 1H), 4.79 (t, 1H), 3.90 (s, 1H), *(2H | |
| | hydroxybenzamide | in solvent peak), 2.06 (d, 2H), | |
| | | 1.59-1.47 (m, 4H) | |
| 13 | N-{cis-4-[1-(3,4- | (500 MHz, DMSO-d₆) δ 9.39 (s, 1H), | 525.2, |
| | difluorophenyl)-6-fluoro-2,4- | 8.55 (d, 1H), 8.26 (dd, 1H), 7.94 (d, | 526.1 |
| | dioxo-1,4-dihydropyrido[2,3- | 1H), 7.59-7.53 (m, 2H), 7.28-7.24 (m, | |
| | d]pyrimidin-3(2H)- | 1H), 6.97 (t, 1H), 6.79 (d, 1H), 6.72 (d, | |
| | yl]cyclohexyl}-3-hydroxy-2- | 1H), 4.74 (t, 1H), 3.92 (s, 1H), | |
| | methylbenzamide | 2.70-2.66 (m, 1H), *(1H in solvent peak), | |
| | | 2.09 (s, 3H), 2.02 (d, 2H), 1.56 (t, 2H), | |
| | | 1.47 (d, 2H) | |
| 14 | N-{cis-4-[1-(3,4- | (500 MHz, DMSO-d₆) δ 9.44 (s, 1H), | 541.1 |
| | difluorophenyl)-6-fluoro-2,4- | 8.56 (d, 1H), 8.29-8.26 (m, 1H), | |
| | dioxo-1,4-dihydropyrido[2,3- | 7.60-7.55 (m, 3H), 7.37 (s, 1H), | |
| | d]pyrimidin-3(2H)- | 7.32-7.25 (m, 2H), 6.77 (d, 1H), 4.82 (t, 1H), | |
| | yl]cyclohexyl}-4-hydroxy-3- | 3.91 (s, 1H), 3.68 (s, 3H), *(2H in solvent | |
| | methoxybenzamide | peak), 2.06 (d, 2H), 1.58-1.46 (m, 4H) | |
| 15 | N-{cis-4-[1-(3,4- | (500 MHz, DMSO-d₆) δ 9.47 (s, 1H), | 525.1 |
| | difluorophenyl)-6-fluoro-2,4- | 8.56 (d, 1H), 8.29 (dd, 1H), 7.64 (d, | |
| | dioxo-1,4-dihydropyrido[2,3- | 1H), 7.60-7.54 (m, 2H), 7.29-7.24 (m, | |
| | d]pyrimidin-3(2H)- | 1H), 7.23-7.20 (m, 2H), 7.09 (d, 1H), | |
| | yl]cyclohexyl}-3-hydroxy-4- | 4.78 (t, 1H), 3.93 (s, 1H), *(2H in | |
| | methylbenzamide | solvent peak), 2.15 (s, 3H), 2.05 (d, 2H), | |
| | | 1.60-1.46 (m, 4H) | |
| 16 | N-{cis-4-[1-(3,4- | (500 MHz, DMSO-d₆) δ 13.90 (s, 1H), | 541.1, |
| | difluorophenyl)-6-fluoro-2,4- | 8.94 (s, 1H), 8.57 (d, 1H), 8.31 (dd, | 542.2 |
| | dioxo-1,4-dihydropyrido[2,3- | 1H), 7.60-7.55 (m, 2H), 7.33-7.28 (m, | |
| | d]pyrimidin-3(2H)- | 2H), 6.59 (d, 1H), 6.49 (d, 1H), 4.83 (t, | |
| | yl]cyclohexyl}-2-hydroxy-6- | 1H), 4.28 (s, 1H), 3.53 (s, 3H), *(2H in | |
| | methoxybenzamide | solvent peak), 1.85 (d, 2H), | |
| | | 1.73-1.61 (m, 4H) | |
| 17 | N-{cis-4-[1-(3,4- | (500 MHz, DMSO-d₆) δ 11.86 (s, 1H), | 541.1 |
| | difluorophenyl)-6-fluoro-2,4- | 8.55 (d, 1H), 8.36-8.27 (m, 2H), | |
| | dioxo-1,4-dihydropyrido[2,3- | 7.60-7.48 (m, 3H), 7.26 (d, 1H), 7.08 (d, 1H), | |
| | d]pyrimidin-3(2H)- | 6.81 (t, 1H), 4.79 (t, 1H), 4.04 (s, 1H), | |
| | yl]cyclohexyl}-2-hydroxy-3- | 3.76 (s, 3H), *(2H in solvent peak), | |
| | methoxybenzamide | 2.02 (s, 2H), 1.67-1.49 (m, 4H) | |
| 18 | 4-chloro-N-{cis-4-[1-(3,4- | (500 MHz, DMSO-d₆) δ | 545 |
| | difluorophenyl)-6-fluoro-2,4- | 12.22-12.20 (m, 1H), 8.56-8.50 (m, 2H), 8.27 (dd, | |
| | dioxo-1,4-dihydropyrido[2,3- | 3.1 Hz, 1H), 7.94 (d, 1H), 7.60-7.54 (m, | |
| | d]pyrimidin-3(2H)- | 2H), 7.29-7.26 (m, 1H), 6.99-6.95 (m, | |
| | yl]cyclohexyl}-2- | 2H), 4.80 (t, 1H), 4.12 (s, 1H), *(2H in | |
| | hydroxybenzamide | solvent peak), 1.95 (d, 2H), | |
| | | 1.69-1.55 (m, 4H) | |
| 19 | N-{cis-4-[1-(3,4- | (500 MHz, DMSO-d₆) δ | 541.1 |
| | difluorophenyl)-6-fluoro-2,4- | 12.27-12.25 (m, 1H), 8.56 (d, 1H), 8.29-8.23 (m, | |
| | dioxo-1,4-dihydropyrido[2,3- | 2H), 7.88 (d, 1H), 7.60-7.55 (m, 2H), | |
| | d]pyrimidin-3(2H)- | 7.28-7.26 (m, 1H), 6.48-6.45 (m, 1H), | |
| | yl]cyclohexyl}-2-hydroxy-4- | 6.42 (t, 1H), 4.79 (t, 1H), 4.06 (s, 1H), | |
| | methoxybenzamide | 3.73 (s, 3H), *(2H in solvent peak), | |
| | | 1.99 (d, 2H), 1.65-1.53 (m, 4H) | |
| 20 | N-{cis-4-[1-(3,4- | (500 MHz, DMSO-d₆) δ | 529.1, |
| | difluorophenyl)-6-fluoro-2,4- | 11.70-11.68 (m, 1H), 8.67-8.62 (m, 1H), 8.56 (d, | 530.2 |
| | dioxo-1,4-dihydropyrido[2,3- | 1H), 8.27 (dd, 1H), 7.71-7.67 (m, 1H), | |
| | d]pyrimidin-3(2H)- | 7.60-7.54 (m, 2H), 7.29-7.19 (m, 2H), | |
| | yl]cyclohexyl}-5-fluoro-2- | 6.96-6.93 (m, 1H), 4.80 (t, 1H), 4.12 (s, | |
| | hydroxybenzamide | 1H), *(2H in solvent peak), 1.95 (d, | |
| | | 2H), 1.68-1.57 (m, 4H) | |
| 21 | (2S)-N-{cis-4-[1-(3,4- | (500 MHz, DMSO-d₆) δ 8.56 (d, 1H), | 505.1 |
| | difluorophenyl)-6-fluoro-2,4- | 8.28 (dd, 1H), 7.60-7.54 (m, 2H), | |
| | dioxo-1,4-dihydropyrido[2,3- | 7.35 (d, 1H), 7.29-7.25 (m, 1H), 4.76 (t, 1H), | |
| | d]pyrimidin-3(2H)- | 3.91 (s, 1H), 3.85 (dd, 1H), *(2H in | |
| | yl]cyclohexyl}-2-hydroxy-4- | solvent peak), 1.78-1.70 (m, 3H), | |
| | methylpentanamide | 1.61-1.51 (m, 4H), 1.45-1.39 (m, 1H), | |
| | | 1.37-1.30 (m, 1H), 0.85-0.82 (m, 6H) | |
| 22 | (2R)-2-cyclohexyl-N-{cis-4-[1- | (500 MHz, DMSO-d₆) δ 8.56 (d, 1H), | 531.2 |
| | (3,4-difluorophenyl)-6-fluoro- | 8.29 (dd, 1H), 7.60-7.54 (m, 2H), | |
| | 2,4-dioxo-1,4- | 7.35 (d, 1H), 7.27-7.25 (m, 1H), 4.76 (t, 1H), | |
| | dihydropyrido[2,3- | 3.93 (s, 1H), 3.66 (d, 1H), *(2H in | |
| | d]pyrimidin-3(2H)- | solvent peak), 1.74 (t, 2H), | |
| | yl]cyclohexyl}-2- | 1.66-1.49 (m, 9H), 1.39-1.30 (m, 1H), | |
| | hydroxyacetamide | 1.21-0.94 (m, 5H) | |
| 23 | methyl 4-({cis-4-[1-(3,4- | (500 MHz, DMSO-d₆) δ 8.56 (d, 1H), | 505.1 |
| | difluorophenyl)-6-fluoro-2,4- | 8.27 (dd, 1H), 7.70 (d, 1H), | |
| | dioxo-1,4-dihydropyrido[2,3- | 7.60-7.54 (m, 2H), 7.28-7.25 (m, 1H), 4.70 (t, | |
| | d]pyrimidin-3(2H)- | 1H), 3.74 (s, 1H), 3.53 (s, 3H), *(6H in | |
| | yl]cyclohexyl}amino)-4- | solvent peak), 1.83 (d, 2H), | |
| | oxobutanoate | 1.51-1.43 (m, 4H) | |
| 24 | N-[2-({cis-4-[1-(3,4- | (500 MHz, DMSO-d₆) δ | 568.1 |
| | difluorophenyl)-6-fluoro-2,4- | 12.15-12.14 (m, 1H), 8.99-8.95 (m, 1H), 8.56 (d, | |
| | dioxo-1,4-dihydropyrido[2,3- | 1H), 8.23-8.20 (m, 1H), 7.87-7.83 (m, | |
| | d]pyrimidin-3(2H)- | 2H), 7.60-7.54 (m, 2H), 7.36 (t, 1H), | |
| | yl]cyclohexyl}amino)-2- | 7.29-7.25 (m, 1H), 6.89-6.83 (m, 2H), | |
| | oxoethyl]-2-hydroxybenzamide | 4.73 (t, 1H), 3.96 (d, 2H), 3.84 (s, 1H), | |
| | | *(2H in solvent peak), 1.85 (d, 2H), | |
| | | 1.57-1.47 (m, 4H) | |
| 25 | methyl 4-[({cis-4-[1-(3,4- | (500 MHz, DMSO-d₆) δ 8.56 (d, 1H), | 553.1 |
| | difluorophenyl)-6-fluoro-2,4- | 8.30 (dd, 3.0 Hz, 1H), 8.16 (d, 1H), | |
| | dioxo-1,4-dihydropyrido[2,3- | 8.00 (d, 2H), 7.94 (d, 2H), 7.60-7.54 (m, | |
| | d]pyrimidin-3(2H)- | 2H), 7.29-7.25 (m, 1H), 4.79 (t, 1H), | |
| | yl]cyclohexyl}amino)carbonyl]- | 3.96 (s, 1H), 3.85 (s, 3H), 2.72-2.66 (m, | |
| | benzoate | 1H), *(1H in solvent peak), 2.09 (d, | |
| | | 2H), 1.63-1.46 (m, 4H) | |
| 26 | N-{cis-4-[1-(3,4- | (500 MHz, DMSO-d₆) δ 9.12 (s, 1H), | 541.1 |
| | difluorophenyl)-6-fluoro-2,4- | 8.55 (d, 1H), 8.29 (dd, 1H), | |
| | dioxo-1,4-dihydropyrido[2,3- | 7.60-7.54 (m, 3H), 7.32 (dd, 1H), 7.29-7.26 (m, | |
| | d]pyrimidin-3(2H)- | 2H), 6.93 (d, 1H), 4.78 (t, 1H), 3.92 (s, | |
| | yl]cyclohexyl}-3-hydroxy-4- | 1H), 3.78 (s, 3H), *(2H in solvent peak), | |
| | methoxybenzamide | 2.05 (d, 2H), 1.58-1.48 (m, 4H) | |
| 27 | methyl 5-({cis-4-[1-(3,4- | (500 MHz, DMSO-d₆) δ 8.55 (d, 1H), | 519.1 |
| | difluorophenyl)-6-fluoro-2,4- | 8.26 (dd, 1H), 7.63 (d, 1H), | |
| | dioxo-1,4-dihydropyrido[2,3- | 7.60-7.54 (m, 2H), 7.28-7.25 (m, 1H), 4.70 (t, | |
| | d]pyrimidin-3(2H)- | 1H), 3.75 (s, 1H), 3.53 (s, 3H), *(2H in | |
| | yl]cyclohexyl}amino)-5- | solvent peak), 2.26 (t, 2H), 2.13 (t, 2H), | |
| | oxopentanoate | 1.84 (d, 2H), 1.70 (quintet, 2H), | |
| | | 1.52-1.43 (m, 4H) | |
| 28 | N-{cis-4-[1-(3,4- | (500 MHz, DMSO-d₆) δ 8.56 (d, 1H), | 525.1, |
| | difluorophenyl)-6-fluoro-2,4- | 8.28 (dd, 1H), 7.87 (d, 1H), | 527.1 |
| | dioxo-1,4-dihydropyrido[2,3- | 7.60-7.55 (m, 2H), 7.29-7.26 (m, 1H), 7.00 (t, | |
| | d]pyrimidin-3(2H)- | 1H), 6.68 (d, 1H), 6.65 (s, 1H), | |
| | yl]cyclohexyl}-2-(3- | 6.54 (dd, 1.7 Hz, 1H), 4.71 (t, 1H), 3.76 (s, | |
| | hydroxyphenyl)acetamide | 1H), *(4H in solvent peak), 1.84 (d, | |
| | | 2H), 1.53-1.44 (m, 4H) | |
| 29 | methyl 3-[({cis-4-[1-(3,4- | (500 MHz, DMSO-d₆) δ 8.56 (d, 1H), | 553.1 |
| | difluorophenyl)-6-fluoro-2,4- | 8.39 (s, 1H), 8.29 (dd, 1H), 8.18 (d, | |
| | dioxo-1,4-dihydropyrido[2,3- | 1H), 8.08 (dd, 2H), 7.61-7.54 (m, 3H), | |
| | d]pyrimidin-3(2H)- | 7.28-7.26 (m, 1H), 4.80 (t, 1H), 3.96 (s, | |
| | yl]cyclohexyl}amino)carbonyl]- | 1H), 3.83 (s, 3H), *(1H in solvent peak), | |
| | benzoate | 2.72-2.66 (m, 1H), 2.10 (d, 2H), 1.59 (t, | |
| | | 2H), 1.51 (d, 2H) | |
| 30 | N-{cis-4-[1-(3,4- | (500 MHz, DMSO-d₆) δ 11.29 (s, 1H), | 529.1 |
| | difluorophenyl)-6-fluoro-2,4- | 8.55 (d, 1H), 8.27 (dd, 3.0 Hz, 1H), | |
| | dioxo-1,4-dihydropyrido[2,3- | 8.00 (t, 1H), 7.59-7.53 (m, 2H), | |
| | d]pyrimidin-3(2H)- | 7.29-7.23 (m, 2H), 6.71-6.66 (m, 2H), 4.76 (t, | |
| | yl]cyclohexyl}-2-fluoro-6- | 1H), 4.07 (s, 1H), *(2H in solvent peak), | |
| | hydroxybenzamide | 1.96 (d, 2H), 1.62 (t, 2H), 1.53 (d, 2H) | |
| 31 | N-{cis-4-[1-(3,4- | (500 MHz, DMSO-d₆) δ 8.55 (d, 1H), | 539.1 |
| | difluorophenyl)-6-fluoro-2,4- | 8.27 (dd, 1H), 7.64 (d, 1H), | |
| | dioxo-1,4-dihydropyrido[2,3- | 7.59-7.53 (m, 2H), 7.28-7.26 (m, 1H), 6.99 (t, | |
| | d]pyrimidin-3(2H)- | 1H), 6.59-6.56 (m, 2H), 6.51 (dd, 1H), | |
| | yl]cyclohexyl}-3-(3- | 4.70 (t, 1H), 3.77 (s, 1H), 2.69-2.66 (m, | |
| | hydroxyphenyl)propanamide | 1H), *(4H in solvent peak), | |
| | | 2.39-2.36 (m, 1H), 1.83 (d, 2H), 1.53-1.42 (m, | |
| | | 4H) | |
| 32 | N-{cis-4-[1-(3,4- | (500 MHz, DMSO-d₆) δ 13.54 (s, 1H), | 535.1 |
| | difluorophenyl)-6-fluoro-2,4- | 8.56 (d, 1H), 8.31 (dd, 1H), 8.14 (d, | |
| | dioxo-1,4-dihydropyrido[2,3- | 1H), 7.60-7.55 (m, 4H), 7.39 (dd, 1H), | |
| | d]pyrimidin-3(2H)- | 7.29-7.27 (m, 1H), 7.21 (t, 1H), 4.83 (t, | |
| | yl]cyclohexyl}-1H-indazole-3- | 1H), 4.20 (s, 1H), *(2H in solvent peak), | |
| | carboxamide | 1.98 (d, 2H), 1.68 (t, 2H), 1.61 (d, 2H) | |
| 33 | N-{cis-4-[1-(3,4- | (500 MHz, DMSO-d₆) δ 8.65 (d, 1H), | 535.1 |
| | difluorophenyl)-6-fluoro-2,4- | 8.56 (d, 1H), 8.49 (s, 1H), 8.30 (dd, | |
| | dioxo-1,4-dihydropyrido[2,3- | 1H), 7.90 (s, 1H), 7.68 (d, 1H), | |
| | d]pyrimidin-3(2H)- | 7.60-7.54 (m, 2H), 7.46 (t, 1H), | |
| | yl]cyclohexyl}imidazo[1,2- | 7.28-7.26 (m, 1H), 7.08 (t, 1H), 4.81 (t, 1H), | |
| | a]pyridine-2-carboxamide | 4.11 (s, 1H), *(2H in solvent peak), 2.00 (d, | |
| | | 2H), 1.68 (t, 2H), 1.59 (d, 2H) | |
| 34 | 5-chloro-N-{cis-4-[6-fluoro-1- | (500 MHz, DMSO-d₆) δ 12.00 (s, 1H), | 555.1, |
| | [3-(methylthio)phenyl]-2,4- | 8.64 (d, 1H), 8.54 (d, 1H), 8.24 (dd 1H), | 557.1 |
| | dioxo-1,4-dihydropyrido[2,3- | 7.93 (d, 1H), 7.42 (t, 1H), 7.37 (dd, 1H), | |
| | d]pyrimidin-3(2H)- | 7.30 (d, 1H), 7.27 (t, 1H), 7.11 (dd, 1H), | |
| | yl]cyclohexyl}-2- | 6.95 (d, 1H), 4.80 (t, 1H), 4.12 (s, 1H), | |
| | hydroxybenzamide | *(5H in solvent peak), 1.95 (d, 2H), | |
| | | 1.68-1.57 (m, 4H) | |
| 35 | N-{cis-4-[6-fluoro-1-[3- | (500 MHz, DMSO-d₆) δ 11.51 (s, 1H), | 535.1 |
| | (methylthio)phenyl]-2,4-dioxo- | 8.54 (d, 1H), 8.52 (d, 1H), 8.24 (dd, | |
| | 1,4-dihydropyrido[2,3- | 1H), 7.72 (d, 1H), 7.42 (t, 1H), | |
| | d]pyrimidin-3(2H)- | 7.31-7.29 (m, 1H), 7.27 (t, 1H), | |
| | yl]cyclohexyl}-2-hydroxy-5- | 7.14-7.10 (m, 2H), 6.80 (d, 1H), 4.81 (t, 1H), | |
| | methylbenzamide | 4.11 (s, 1H), *(5H in solvent peak), 2.18 (s, | |
| | | 3H), 1.95 (d, 2H), 1.67-1.56 (m, 4H) | |
| 36 | N-{cis-4-[6-fluoro-1-[3- | (500 MHz, DMSO-d₆) δ 11.61 (d, 1H), | 533.2, |
| | (methylthio)phenyl]-2,4-dioxo- | 8.59 (d, 1H), 8.54 (d, 1H), 8.24 (dd, | 551.2 |
| | 1,4-dihydropyrido[2,3- | 1H), 7.88 (d, 1H), 7.42 (t, 1H), | |
| | d]pyrimidin-3(2H)- | 7.31-7.26 (m, 3H), 7.11 (dd, 1H), 6.88 (d, | |
| | yl]cyclohexyl}-2-hydroxy-5- | 1H), 5.06 (t, 1H), 4.80 (t, 1H), 4.37 (d, | |
| | (hydroxymethyl)benzamide | 2H), 4.13 (s, 1H), *(5H in solvent peak), | |
| | | 1.95 (d, 2H), 1.68-1.57 (m, 4H) | |
| 37 | (2R)-N-{cis-4-[6-fluoro-1-[3- | (500 MHz, DMSO-d₆) δ 8.55 (d, 1H), | 535.1, |
| | (methylthio)phenyl]-2,4-dioxo- | 8.28 (dd, 1H), 7.60 (d, 1H), | 536.2 |
| | 1,4-dihydropyrido[2,3- | 7.45-7.39 (m, 3H), 7.31-7.19 (m, 5H), 7.12 (dd, | |
| | d]pyrimidin-3(2H)- | 1H), 4.92 (s, 1H), 4.77 (t, 1H), 3.87 (s, | |
| | yl]cyclohexyl}-2-hydroxy-2- | 1H), *(5H in solvent peak), | |
| | phenylacetamide | 1.82-1.72 (m, 2H), 1.60-1.53 (m, 4H) | |
| 38 | (2S)-N-{cis-4-[6-fluoro-1-[3- | (500 MHz, DMSO-d₆) δ 8.55 (d, 1H), | 535.1 |
| | (methylthio)phenyl]-2,4-dioxo- | 8.28 (dd, 1H), 7.60 (d, 1H), | |
| | 1,4-dihydropyrido[2,3- | 7.45-7.39 (m, 3H), 7.32-7.19 (m, 5H), 7.13 (d, | |
| | d]pyrimidin-3(2H)- | 1H), 4.92 (s, 1H), 4.77 (t, 1H), 3.87 (s, | |
| | yl]cyclohexyl}-2-hydroxy-2- | 1H), *(5H in solvent peak), | |
| | phenylacetamide | 1.81-1.73 (m, 2H), 1.60-1.52 (m, 4H) | |
| 39 | N-{cis-4-[6-fluoro-1-[3- | (500 MHz, DMSO-d₆) δ 8.54 (d, 1H), | 459.1 |
| | (methylthio)phenyl]-2,4-dioxo- | 8.26 (dd, 1H), 7.42 (t, 1H), | |
| | 1,4-dihydropyrido[2,3- | 7.31-7.26 (m, 3H), 7.11 (dd, 1H), 4.77 (t, 1H), | |
| | d]pyrimidin-3(2H)- | 3.96 (s, 1H), 3.78 (s, 2H), *(5H in | |
| | yl]cyclohexyl}-2- | solvent peak), 1.77 (d, 2H), | |
| | hydroxyacetamide | 1.61-1.53 (m, 4H) | |
| 40 | 5-chloro-N-{cis-4-[6-fluoro-1- | (500 MHz, DMSO-d₆) δ 12.01 (s, 1H), | 587 |
| | [3-(methylsulfonyl)phenyl]- | 8.63 (d, 1H), 8.53 (d, 1H), 8.27 (dd, | |
| | 2,4-dioxo-1,4- | 1H), 8.02-7.99 (m, 2H), 7.94 (d, 1H), | |
| | dihydropyrido[2,3- | 7.81-7.74 (m, 2H), 7.37 (dd, 1H), | |
| | d]pyrimidin-3(2H)- | 6.95 (d, 1H), 4.80 (t, 1H), 4.11 (s, 1H), *(5H | |
| | yl]cyclohexyl}-2- | in solvent peak), 1.96 (d, 2H), | |
| | hydroxybenzamide | 1.68-1.58 (m, 4H) | |
| 41 | N-{cis-4-[6-fluoro-1-[3- | (500 MHz, DMSO-d₆) δ 11.64 (s, 1H), | 565.1, |
| | (methylsulfonyl)phenyl]-2,4- | 8.58 (d, 1H), 8.53 (d, 1H), 8.27 (dd, | 583.1 |
| | dioxo-1,4-dihydropyrido[2,3- | 1H), 8.02-7.99 (m, 2H), 7.88 (d, 1H), | |
| | d]pyrimidin-3(2H)- | 7.81-7.74 (m, 2H), 7.27 (dd, 1H), | |
| | yl]cyclohexyl}-2-hydroxy-5- | 6.87 (d, 1H), 4.80 (t, 1H), 4.37 (d, 2H), | |
| | (hydroxymethyl)benzamide | 4.13 (s, 1H), *(5H in solvent peak), 1.96 (d, | |
| | | 2H), 1.68-1.59 (m, 4H) | |
| 42 | (2R)-N-{cis-4-[6-fluoro-1-[3- | (500 MHz, DMSO-d₆) δ 8.54 (d, 1H), | 567.1 |
| | (methylsulfonyl)phenyl]-2,4- | 8.31 (dd, 1H), 8.03-8.01 (m, 2H), | |
| | dioxo-1,4-dihydropyrido[2,3- | 7.82-7.75 (m, 2H), 7.60 (d, 1H), 7.40 (d, 2H), | |
| | d]pyrimidin-3(2H)- | 7.27-7.19 (m, 3H), 4.92 (s, 1H), 4.77 (t, | |
| | yl]cyclohexyl}-2-hydroxy-2- | 1H), 3.87 (s, 1H), *(5H in solvent peak), | |
| | phenylacetamide | 1.80-1.74 (m, 2H), 1.61-1.52 (m, 4H) | |
| 43 | (2S)-N-{cis-4-[6-fluoro-1-[3- | (500 MHz, DMSO-d₆) δ 8.54 (d, 1H), | 567.2 |
| | (methylsulfonyl)phenyl]-2,4- | 8.31 (dd, 1H), 8.03-8.01 (m, 2H), | |
| | dioxo-1,4-dihydropyrido[2,3- | 7.82-7.75 (m, 2H), 7.60 (d, 1H), 7.40 (d, 2H), | |
| | d]pyrimidin-3(2H)- | 7.27-7.19 (m, 3H), 4.92 (s, 1H), 4.77 (t, | |
| | yl]cyclohexyl}-2-hydroxy-2- | 1H), 3.87 (d, 1H), *(5H in solvent | |
| | phenylacetamide | peak), 1.80-1.73 (m, 2H), 1.59-1.52 (m, | |
| | | 4H) | |
| 44 | N-{cis-4-[6-fluoro-1-[3- | (500 MHz, DMSO-d₆) δ 8.53 (d, 1H), | 491.2 |
| | (methylsulfonyl)phenyl]-2,4- | 8.29 (dd, 1H), 8.02-7.98 (m, 2H), | |
| | dioxo-1,4-dihydropyrido[2,3- | 7.81-7.73 (m, 2H), 7.29 (d, 1H), 4.77 (t, 1H), | |
| | d]pyrimidin-3(2H)- | 3.96 (s, 1H), 3.78 (s, 2H), *(5H in | |
| | yl]cyclohexyl}-2- | solvent peak), 1.77 (d, 2H), | |
| | hydroxyacetamide | 1.62-1.55 (m, 4H) | |
| 45 | methyl 3-[6-fluoro-3-{cis-4- | (400 MHz, DMSO-d₆) δ 8.55 (d, 1H), | 547.1 |
| | [(2-hydroxy-5- | 8.50 (d, 1H), 8.29 (dd, 1H), | |
| | methylbenzoyl)amino]cyclohexyl}- | 8.05-8.02 (m, 2H), 7.75 (s, 1H), 7.69 (d, 2H), | |
| | 2,4-dioxo-3,4- | 7.16 (d, 1H), 6.83 (d, 1H), 4.85 (t, 1H), | |
| | dihydropyrido[2,3- | 4.14 (s, 1H), 3.86 (s, 3H), 2.68-2.55 (m, 2H), | |
| | d]pyrimidin-1(2H)-yl]benzoate | 2.20 (s, 3H), 1.99 (d, 2H), 1.70-1.61 (m, 4H) | |

### EXAMPLE 46

### N-{cis-4-[1-(3,4-Difluorophenyl)-6-fluoro-2-oxido-4-oxo-1,4-dihydro-3H-pyrido[2,3-c][1,2,6]thiadiazin-3-yl]cyclohexyl}-2-hydroxy-5-methylbenzamide

The title product was prepared according to Example 1, Step b starting from 3-(cis-4-aminocyclohexyl)-1-(3,4-difluorophenyl)-6-fluoro-1H-pyrido[2,3-c][1,2,6]thiadiazin-4(3H)-one 2-oxide.

¹H NMR (400 MHz, DMSO-d₆) δ 11.69 (s, 1H), 8.67 (d, 1H), 8.52-8.45 (m, 1H), 8.40 (dd, 1H), 7.71-7.63 (m, 3H), 7.39-7.35 (m, 1H), 7.18 (d, 1H), 6.82 (d, 1H), 4.54 (t, 1H), 4.13 (s, 1H), 2.24 (s, 3H), 2.12-1.93 (m, 5H), 1.84-1.74 (m, 3H)

APCI-MS m/z: 545.1 [MH⁺].

### EXAMPLE 47

### N-{cis-4-[1-(3,4-Difluorophenyl)-6-fluoro-2,4-dioxo-1,4-dihydropyrido[2,3-d]pyrimidin-3 (2H)-yl]cyclohexyl}methanesulfonamide

### Step a: 3-(cis-4-Aminocyclohexyl)-1-(3,4-difluorophenyl)-6-fluoropyrido[2,3-d]pyrimidine-2,4(1H,3H)-dione hydrochloride

A mixture of tert-butyl {cis-4-[1-(3,4-difluorophenyl)-6-fluoro-2,4-dioxo-1,4-dihydropyrido[2,3-d]pyrimidin-3(2H)-yl]cyclohexyl}carbamate (98 mg, 0.2 mmol) and 4M HCl in 1,4-dioxane (2 mL) was stirred at room temperature for one hour. The solvents were removed and the pure crude product was used directly.

APCI-MS m/z: 391 [MH⁺].

### Step b: N-{cis-4-[1-(3,4-Difluorophenyl)-6-fluoro-2,4-dioxo-1,4-dihydropyrido[2,3-d]pyrimidin-3(2H)-yl]cyclohexyl}methanesulfonamide

3-(cis-4-Aminocyclohexyl)-1-(3,4-difluorophenyl)-6-fluoropyrido[2,3-d]pyrimidine-2,4(1H,3H)-dione hydrochloride (25 mg, 0.051 mmol) was dissolved in pyridine (500 μl) and methanesulfonyl chloride (150 μl, 1.93 mmol) was added. The mixture was stirred at 60° C. and product was formed after 1 h. The title compound (8 mg, 33%) was obtained after purification by preparative HPLC.

¹H NMR (400 MHz, DMSO-d₆) δ 8.59 (d, 1H), 8.30 (dd, 1H), 7.64-7.57 (m, 2H), 7.32-7.29 (m, 1H), 4.74 (t, 1H), 3.51 (s, 1H), 2.90 (s, 3H), 2.73-2.64 (m, 2H), 1.91 (d, 2H), 1.58 (t, 2H), 1.48 (d, 2H)

APCI-MS m/z: 469 [MH⁺].

### EXAMPLE 48 TO 76

The following compounds were synthesised in analogy to the procedure described in Example 1.

| Ex | Compound | ¹H NMR | m/z |
|---|---|---|---|
| 48 | N-{cis-4-[1-(3,4- | (400 MHz, DMSO-d₆): δ | 459 |
| | Difluorophenyl)-6-fluoro-2,4- | 8.60 (1H, d); 8.32 (1H, dd); 7.88 (1H, | |
| | dioxo-1,4-dihydropyrido[2,3- | d); 7.65-7.57 (2H, m); 7.31 (1H, | |
| | d]pyrimidin-3(2H)- | m); 4.75 (1H, t); 3.78 (1H, s); | |
| | yl]cyclohexyl}cyclopropanecarboxamide | 2.64 (2H, q); 1.90 (2H, d); | |
| | | 1.76 (1H, m) 1.59-1.46 (4H, m); | |
| | | 0.63 (4H, m). | |
| 49 | Methyl 3-[6-fluoro-3-{cis-4- | (400 MHz, DMSO-d₆): δ | 524 |
| | [(1-methyl-L- | 8.55 (1H, d); 8.29 (1H, dd); 8.04 (1H, | |
| | prolyl)amino]cyclohexyl}-2,4- | dt); 8.01 (1H, brs); 7.69 (2H, | |
| | dioxo-3,4-dihydropyrido[2,3- | brd); 7.65 (1H, brd); 4.78 (1H, | |
| | d]pyrimidin-1(2H)- | brt); 3.90 (1H, brs); 3.87 (3H, s); | |
| | yl]benzoate | 3.05 (1H, brs); 2.74 (1H, m); | |
| | | *(2H in solvent peak); 2.33 (3H, | |
| | | s); 2.27 (1H, q); 2.07 (1H, m); | |
| | | 1.83 (2H, t); 1.73-1.54 (7H, m). | |
| 50 | methyl 5-[({cis-4-[1-(3,4- | (500 MHz, DMSO-d₆): δ | 554.1 |
| | difluorophenyl)-6-fluoro-2,4- | 9.15 (1H, d); 8.60 (1H, d); 8.50 (1H, | |
| | dioxo-1,4-dihydropyrido[2,3- | dd); 8.42 (1H, d); 8.35 (1H, dd); | |
| | d]pyrimidin-3(2H)- | 8.20 (1H, d); 7.64-7.58 (2H, m); | |
| | yl]cyclohexyl}amino)carbonyl] | 7.31 (1H, d); 4.86 (1H, t); | |
| | pyridine-2-carboxylate | 4.18 (1H, s); 3.93 (3H, s); 2.59 (2H, | |
| | | d); 2.02 (2H, d); 1.74 (2H, s); | |
| | | 1.64 (2H, d). | |
| 51 | N-{cis-4-[1-(3,4- | (400 MHz, DMSO-d₆): δ | 529.1 |
| | difluorophenyl)-6-fluoro-2,4- | 8.60 (1H, d); 8.31 (1H, m); | |
| | dioxo-1,4-dihydropyrido[2,3- | 8.05-7.98; (1H, m); 7.65-7.57 (2H, | |
| | d]pyrimidin-3(2H)- | m); 7.32-7.29 (1H, m); | |
| | yl]cyclohexyl}-4-fluoro-2- | 6.73 (2H, s); 4.83 (1H, t); 4.13 (1H, | |
| | hydroxybenzamide | s); 2.61 (2H, d); 2.00 (2H, d); | |
| | | 1.71-1.59 (4H, m). | |
| 52 | N-{cis-4-[1-(3,4- | (400 MHz, DMSO-d₆): δ | 475.1 |
| | difluorophenyl)-6-fluoro-2,4- | 8.60 (1H, d); 8.33 (1H, dd); | |
| | dioxo-1,4-dihydropyrido[2,3- | 7.64-7.57 (2H, m); 7.41 (1H, d); | |
| | d]pyrimidin-3(2H)- | 7.32-7.29 (1H, m); 4.82 (1H, t); | |
| | yl]cyclohexyl}-1- | 4.00 (1H, s); 1.81-1.78 (2H, m); | |
| | hydroxycyclopropanecarboxamide | 1.68-1.59 (4H, m); 1.03 (2H, q); | |
| | | 0.85 (2H, q). | |
| 53 | N-{cis-4-[1-(3,4- | (400 MHz, DMSO-d₆): δ | 546.1 |
| | difluorophenyl)-6-fluoro-2,4- | 11.91 (1H, d); 9.21 (1H, s); 8.66 (1H, | |
| | dioxo-1,4-dihydropyrido[2,3- | d); 8.62-8.59 (2H, m); 8.41 (1H, | |
| | d]pyrimidin-3(2H)- | dd); 8.22 (1H, d); 7.77 (1H, t); | |
| | yl]cyclohexyl}quinoline-8- | 7.68-7.60 (3H, m); | |
| | carboxamide | 7.37-7.34 (1H, m); 4.90 (1H, t); 4.47 (1H, | |
| | | s); 2.90-2.80 (2H, m); | |
| | | 1.94-1.90 (2H, m); 1.79-1.70 (4H, | |
| | | m). | |
| 54 | N-{cis-4-[6-fluoro-1-[3- | (400 MHz, DMSO-d₆): δ | 495.1 |
| | (methylthio)phenyl]-2,4- | 8.58 (1H, d); 8.30 (1H, dd); 7.84 (1H, | |
| | dioxo-1,4-dihydropyrido[2,3- | s); 7.54 (1H, d); 7.45 (1H, t); | |
| | d]pyrimidin-3(2H)- | 7.35-7.30 (2H, m); | |
| | yl]cyclohexyl}-2-furamide | 7.17-7.13 (2H, m); 6.63-6.62 (1H, m); | |
| | | 4.82 (1H, t); 4.00 (1H, s); | |
| | | 2.67-2.61 (2H, m); 2.47 (3H, s); | |
| | | 2.05 (2H, d); 1.66-1.54 (4H, m). | |
| 55 | N-{cis-4-[6-fluoro-1-[3- | (400 MHz, DMSO-d₆): δ | 469.1 |
| | (methylthio)phenyl]-2,4- | 8.58 (1H, d); 8.29 (1H, dd); 7.86 (1H, | |
| | dioxo-1,4-dihydropyrido[2,3 | d); 7.45 (1H, t); 7.35-7.30 (2H, | |
| | d]pyrimidin-3(2H)- | m); 7.15 (1H, d); 4.76 (1H, t); | |
| | yl]cyclohexyl}cyclopropanecarboxamide | 3.78 (1H, s); 2.69-2.58 (2H, m); | |
| | | 2.47 (3H, s); 1.90 (2H, d); | |
| | | 1.79-1.73 (1H, m); 1.56-1.48 (4H, | |
| | | m); 0.64-0.60 (4H, m). | |
| 56 | N-{cis-4-[6-fluoro-1-[3- | (400 MHz, DMSO-d₆): δ | 457.1 |
| | (methylthio)phenyl]-2,4- | 8.58 (1H, d); 8.28 (1H, dd); 7.45 (1H, | |
| | dioxo-1,4-dihydropyrido[2,3- | t); 7.34-7.30 (2H, m); 7.14 (1H, | |
| | d]pyrimidin-3(2H)- | dd); 4.75 (1H, t); 3.77 (1H, s); | |
| | yl]cyclohexyl}propanamide | 2.65-2.56 (2H, m); 2.47 (3H, s); | |
| | | 2.14 (2H, q); 1.89 (2H, d); | |
| | | 1.55-1.46 (4H, m); 0.99 (3H, t). | |
| 57 | N-{cis-4-[6-fluoro-1-[3- | (400 MHz, DMSO-d₆): δ | 505.1 |
| | (methylthio)phenyl]-2,4- | 8.58 (1H, d); 8.30 (1H, dd); 7.93 (1H, | |
| | dioxo-1,4-dihydropyrido[2,3- | d); 7.86 (2H, d); 7.54-7.44 (4H, | |
| | d]pyrimidin-3(2H)- | m); 7.35-7.30 (2H, m); | |
| | yl]cyclohexyl}benzamide | 7.15 (1H, d); 4.83 (1H, t); 3.98 (1H, | |
| | | s); 2.77-2.67 (2H, m); 2.47 (3H, | |
| | | s); 2.11 (2H, d); 1.65-1.53 (4H, | |
| | | m). | |
| 58 | N-{cis-4-[6-fluoro-1-[3- | (400 MHz, DMSO-d₆): δ | 497.2 |
| | (methylthio)phenyl]-2,4- | 8.58 (1H, d); 8.28 (1H, dd); 7.45 (1H, | |
| | dioxo-1,4-dihydropyrido[2,3 | t); 7.34-7.30 (3H, m); 7.14 (1H, | |
| | d]pyrimidin-3(2H)- | d); 4.74 (1H, t); 3.77 (1H, s); | |
| | yl]cyclohexyl}cyclopentanecarboxamide | 2.72-2.57 (3H, m); 2.47 (3H, s); | |
| | | 1.89 (2H, d); 1.76-1.69 (2H, m); | |
| | | 1.61-1.48 (10H, m). | |
| 59 | N-{cis-4-[6-fluoro-1-[3- | (400 MHz, DMSO-d₆): δ | 487.1 |
| | (methylthio)phenyl]-2,4- | 8.58 (1H, d); 8.30 (1H, dd); 7.46 (1H, | |
| | dioxo-1,4-dihydropyrido[2,3- | t); 7.39 (1H, d); 7.35-7.30 (2H, | |
| | d]pyrimidin-3(2H)- | m); 7.14 (1H, d); 4.80 (1H, t); | |
| | yl]cyclohexyl}-2-hydroxy-2- | 3.92 (1H, s); 2.48 (3H, s); | |
| | methylpropanamide | 1.77 (2H, d); 1.65-1.58 (4H, m); | |
| | | 1.25 (6H, s). | |
| 60 | N-{cis-4-[6-fluoro-1-[3- | (400 MHz, DMSO-d₆): δ | 541.1 |
| | (methylthio)phenyl]-2,4- | 8.58 (1H, d); 8.28 (1H, dd); | |
| | dioxo-1,4-dihydropyrido[2,3- | 7.88-7.86 (2H, m); 7.65-7.56 (4H, | |
| | d]pyrimidin-3(2H)- | m); 7.45 (1H, t); 7.34-7.30 (2H, | |
| | yl]cyclohexyl}benzenesulfonamide | m); 7.16-7.13 (1H, m); | |
| | | 4.69 (1H, t); 3.06 (1H, s); | |
| | | 2.77-2.67 (2H, m); 2.47 (3H, s); 1.74 (2H, | |
| | | d); 1.49-1.42 (4H, m). | |
| 61 | methyl 3-[3-{cis-4- | (400 MHz, DMSO-d₆): δ | 495.1 |
| | [(cyclobutylcarbonyl)amino]cyclohexyl}- | 8.55 (1H, d); 8.29 (1H, dd); | |
| | 6-fluoro-2,4- | 8.06-8.03 (1H, m); 8.01 (1H, s); | |
| | dioxo-3,4-dihydropyrido[2,3- | 7.69-7.67 (2H, m); 4.74 (1H, t); | |
| | d]pyrimidin-1(2H)- | 3.87 (3H, s); 3.77 (1H, s); | |
| | yl]benzoate | 3.17-3.09 (1H, m); 2.59-2.54 (2H, m); | |
| | | 2.15-2.06 (2H, m); | |
| | | 2.03-1.95 (2H, m); 1.91-1.83 (3H, m); | |
| | | 1.78-1.71 (1H, m); | |
| | | 1.55-1.48 (4H, m). | |
| 62 | methyl 3-[6-fluoro-3-[cis-4- | (400 MHz, DMSO-d₆): δ | 483.1 |
| | (isobutyrylamino)cyclohexyl]- | 8.55 (1H, d); 8.30 (1H, dd); | |
| | 2,4-dioxo-3,4- | 8.05-8.01 (2H, m); 7.68 (2H, d); | |
| | dihydropyrido[2,3- | 4.75 (1H, t); 3.87 (3H, s); 3.75 (1H, | |
| | d]pyrimidin-1(2H)- | s); 2.67-2.56 (2H, m); 1.89 (2H, | |
| | yl]benzoate | d); 1.54-1.48 (4H, m); 0.98 (6H, | |
| | | d). | |
| 63 | methyl 3-[6-fluoro-3-{cis-4- | (400 MHz, DMSO-d₆): δ | 497.1 |
| | [(3- | 8.55 (1H, d); 8.29 (1H, dd); | |
| | methylbutanoyl)amino]cyclohexyl}- | 8.06-8.01 (2H, m); 7.68 (2H, d); | |
| | 2,4-dioxo-3,4- | 4.74 (1H, t); 3.87 (3H, s); 3.80 (1H, | |
| | dihydropyrido[2,3- | s); 2.67-2.57 (2H, m); | |
| | d]pyrimidin-1(2H)- | 2.01-1.85 (5H, m); 1.56-1.49 (4H, | |
| | yl]benzoate | m); 0.87 (6H, d). | |
| 64 | methyl 3-[3-{cis-4-[(2,2- | (400 MHz, DMSO-d₆): δ | 497.1 |
| | dimethylpropanoyl)amino]cyclohexyl}- | 8.55 (1H, d); 8.31 (1H, dd); | |
| | 6-fluoro-2,4-dioxo- | 8.06-8.01 (2H, m); 7.69-7.68 (2H, | |
| | 3,4-dihydropyrido[2,3- | m); 6.62 (1H, d); 4.78 (1H, t); | |
| | d]pyrimidin-1(2H)- | 3.87 (3H, s); 3.80 (1H, s); | |
| | yl]benzoate | 2.63-2.53 (2H, m); 1.93-1.90 (2H, | |
| | | m); 1.55-1.49 (4H, m); | |
| | | 1.14 (9H, s). | |
| 65 | methyl 3-[6-fluoro-3-{cis-4- | (400 MHz, DMSO-d₆): δ | 485.1 |
| | [(methoxyacetyl)amino]cyclohexyl}- | 8.55 (1H, d); 8.31 (1H, dd); | |
| | 2,4-dioxo-3,4- | 8.06-8.01 (2H, m); 7.69 (2H, d); | |
| | dihydropyrido[2,3- | 7.25 (1H, d); 4.79 (1H, t); 3.94 (1H, | |
| | d]pyrimidin-1(2H)- | s); 3.87 (3H, s); 3.83 (2H, s); | |
| | yl]benzoate | 3.33 (3H, s); 1.86 (2H, d); | |
| | | 1.63-1.56 (4H, m). | |
| 66 | methyl 3-[6-fluoro-2,4-dioxo- | (400 MHz, DMSO-d₆): δ | 523.1 |
| | 3-{cis-4-[(2- | 8.55 (1H, d); 8.31 (1H, dd); | |
| | thienylcarbonyl)amino]cyclohexyl}- | 8.05-8.01 (2H, m); 7.92-7.88 (2H, | |
| | 3,4-dihydropyrido[2,3- | m); 7.73 (1H, d); 7.68 (2H, d); | |
| | d]pyrimidin-1(2H)- | 7.14 (1H, dd); 4.82 (1H, t); | |
| | yl]benzoate | 3.95 (1H, s); 3.86 (3H, s); | |
| | | 2.74-2.64 (2H, m); 2.12-2.08 (2H, m); | |
| | | 1.64-1.53 (4H, m). | |
| 67 | methyl 3-[3-[cis-4- | (400 MHz, DMSO-d₆): δ | 455.1 |
| | (acetylamino)cyclohexyl]-6- | 8.55 (1H, d); 8.30 (1H, dd); | |
| | fluoro-2,4-dioxo-3,4- | 8.06-8.01 (2H, m); 7.69 (2H, d); | |
| | dihydropyrido[2,3- | 4.74 (1H, t); 3.87 (3H, s); 3.77 (1H, | |
| | d]pyrimidin-1(2H)- | s); 2.62-2.56 (2H, m); | |
| | yl]benzoate | 1.91-1.84 (5H, m); 1.57-1.49 (4H, | |
| | | m). | |
| 68 | methyl 3-[6-fluoro-2,4-dioxo- | (400 MHz, DMSO-d₆): δ | 557.1 |
| | 3-{cis-4-[(pyrazolo[1,5- | 8.74 (1H, d); 8.56 (1H, d); 8.32 (1H, | |
| | a]pyridin-2- | dd); 8.06-8.02 (2H, m); | |
| | ylcarbonyl)amino]cyclohexyl}- | 7.77 (1H, d); 7.70-7.68 (2H, m); | |
| | 3,4-dihydropyrido[2,3- | 7.62 (1H, d); 7.29 (1H, t); | |
| | d]pyrimidin-1(2N)- | 7.04-7.00 (2H, m); 4.85 (1H, t); 4.16 (1H, | |
| | yl]benzoate | s); 3.86 (3H, s); 2.66-2.56 (2H, | |
| | | m); 2.04 (2H, d); 1.74-1.63 (4H, | |
| | | m). | |
| 69 | methyl 3-[6-fluoro-3-{cis-4- | (400 MHz, DMSO-d₆): δ | 557.1 |
| | [(imidazo[1,2-a]pyridin-2- | 8.80 (1H, s); 8.55 (1H, d); 8.31 (1H, | |
| | ylcarbonyl)amino]cyclohexyl}- | dd); 8.15 (1H, s); | |
| | 2,4-dioxo-3,4- | 8.05-8.01 (3H, m); 7.68 (3H, d); 7.13 (1H, | |
| | dihydropyrido[2,3- | s); 4.85 (1H, t); 4.21 (1H, s); | |
| | d]pyrimidin-1(2H)- | 3.86 (3H, s); 2.76-2.69 (2H, m); | |
| | yl]benzoate | 2.10-2.02 (2H, m); 1.76-1.65 (4H, | |
| | | m). | |
| 70 | methyl 3-[3-{cis-4- | (400 MHz, DMSO-d₆): δ | 481.1 |
| | [(cyclopropylcarbonyl)amino] | 8.55 (1H, d); 8.30 (1H, dd); | |
| | cyclohexyl}-6-fluoro-2,4- | 8.06-8.02 (2H, m); 7.86 (1H, d); | |
| | dioxo-3,4-dihydropyrido[2,3- | 7.69 (2H, dd); 4.76 (1H, t); 3.87 (3H, | |
| | d]pyrimidin-1(2H)- | s); 3.78 (1H, s); 2.69-2.58 (2H, | |
| | yl]benzoate | m); 1.90 (2H, d); 1.79-1.71 (1H, | |
| | | m); 1.57-1.49 (4H, m); | |
| | | 0.66-0.58 (4H, m). | |
| 71. | N-{cis-4-[6-fluoro-1-[3- | (400 MHz, DMSO-d₆): δ | 501.1 |
| | (methylsulfonyl)phenyl]-2,4- | 8.58 (m, d); 8.32 (1H, dd); | |
| | dioxo-1,4-dihydropyrido[2,3- | 8.06-8.03 (2H, m); 7.89-7.76 (3H, | |
| | d]pyrimidin-3(2H)- | m); 4.76 (1H, t); 3.79 (1H, s); | |
| | yl]cyclohexyl}cyclopropanecarboxamide | 3.28 (3H, s); 2.69-2.59 (2H, m); | |
| | | 1.90 (2H, d); 1.79-1.73 (1H, m); | |
| | | 1.56-1.49 (4H, m); | |
| | | 0.64-0.60 (4H, m). | |
| 72 | N-{cis-4-{1-(3,4- | (400 MHz, DMSO-d₆): δ | 525.1 |
| | difluorophenyl)-6-fluoro-2,4- | 11.56 (1H, s); 8.60 (1H, d); 8.53 (1H, | |
| | dioxo-1,4-dihydropyrido[2,3- | d); 8.31 (1H, dd); 7.76 (1H, d); | |
| | d]pyrimidin-3(2H)- | 7.65-7.56 (2H, m); | |
| | yl]cyclohexyl}-2-hydroxy-5- | 7.34-7.28 (1H, m); 7.17 (1H, dd); 6.84 (1H, | |
| | methylbenzamide | d); 4.84 (1H, t); 4.14 (1H, s); | |
| | | 2.61 (2H, d); 2.22 (3H, s); | |
| | | 2.00 (2H, d); 1.72-1.58 (4H, m). | |
| 73 | methyl 3-[6-fluoro-3-(cis-4- | (400 MHz, DMSO-d₆): δ | 538.3 |
| | {[(1-methylpiperidin-4- | 8.55 (1H, d); 8.30 (1H, dd); 8.04 (1H, | |
| | yl)carbonyl]amino}cyclohexyl)- | td); 8.01 (1H, d); 7.69 (2H, d); | |
| | 2,4-dioxo-3,4- | 7.55 (1H, d); 4.75 (1H, t); | |
| | dihydropyrido[2,3- | 3.87 (3H, s); 3.76 (1H, s); 2.76 (2H, | |
| | d]pyrimidin-1(2H)- | d); 2.64-2.54 (2H, m); | |
| | yl]benzoate | 2.25-2.16 (1H, m); 2.13 (3H, s); | |
| | | 1.91-1.79 (4H, m); 1.59-1.48 (8H, | |
| | | m). | |
| 74 | methyl 3-[3-[cis-4-({5- | (400 MHz, DMSO-d₆): δ | 564.2 |
| | [(dimethylamino)methyl]-2- | 8.55 (1H, d); 8.30 (1H, dd); | |
| | furoyl}amino)cyclohexyl]-6- | 8.06-8.03 (1H, m); 8.01 (1H, d); | |
| | fluoro-2,4-dioxo-3,4- | 7.69 (2H, d); 7.44 (1H, d); 7.12 (1H, | |
| | dihydropyrido[2,3- | d); 6.40 (1H, d); 4.84 (1H, t); | |
| | d]pyrimidin-1(2H)- | 4.01 (1H, s); 3.86 (3H, s); | |
| | yl]benzoate | 3.43 (2H, s); 2.66-2.56 (2H, m); | |
| | | 2.12 (6H, s); 2.02 (2H, d); | |
| | | 1.65-1.56 (4H, m). | |
| 75 | methyl 3-[3-{cis-4- | (400 MHz, DMSO-d₆): δ | 523.2 |
| | [(cyclohexylcarbonyl)amino]cyclohexyl}- | 8.55 (1H, d); 8.30 (1H, dd); | |
| | 6-fluoro-2,4- | 8.06-8.03 (1H, m); 8.01 (1H, s); | |
| | dioxo-3,4-dihydropyrido[2,3- | 7.69 (2H, d); 7.46 (1H, d); 4.74 (1H, | |
| | d]pyrimidin-1(2H)- | t); 3.87 (3H, s); 3.75 (1H, s); | |
| | yl]benzoate | 2.66-2.54 (2H, m); 2.25 (1H, t); | |
| | | 1.87 (2H, d); 1.70-1.59 (5H, m); | |
| | | 1.54-1.48 (4H, m); 1.35-1.10 (5H, | |
| | | m). | |

### EXAMPLE 76

### 1-(3,4-difluorophenyl)-6-fluoro-3-[1-(1H-indazol-3-ylcarbonyl)piperidin-4-yl]pyrido[2,3-d]pyrimidine-2,4(1H,3H)-dione

A mixture of 1-(3,4-difluorophenyl)-6-fluoro-3-piperidin-4-ylpyrido[2,3-d]pyrimidine-2,4(1H,3H)-dione hydrochloride (0.104 mmol), 1H-indazole-3-carboxylic acid (34 mg, 0.210 mmol), HATU (80 mg, 0.210 mmol), HOAT (29 mg, 0.210 mmol) and DIEA (89 μl, 0.520 mmol) in NMP (1 ml) was stirred for 2 h at room temperature. The title compound was obtained by purification on RP-HPLC (18 mg, 33%).

¹H NMR (400 MHz, DMSO-d₆): δ 8.60 (1H, d); 8.33 (1H, dd); 7.96 (1H, d); 7.64-7.57 (3H, m); 7.42 (1H, t); 7.32-7.28 (1H, m); 7.22 (1H, t); 5.13 (1H, t); 4.93-4.84 (1H, m); 4.79-4.72 (1H, m); 3.29-3.21 (1H, m); 2.96-2.85 (1H, m); 2.63-2.52 (1H, m); 1.86-1.72 (2H, m).

APCI-MS m/z: 521.1 [MH⁺].

LC (method A) rt=10.6 min

### EXAMPLES 77 TO 78

The following compounds were synthesised in an analogous method to Example 49.

| Ex | Compound | ¹H NMR | m/z |
|---|---|---|---|
| 77 | 1-(3,4-difluorophenyl)-6-fluoro- | (400 MHz, DMSO-d₆) δ | 511.1 |
| | 3-{1-[(2R)-2-hydroxy-2- | 8.59 (1H, s); 8.31-8.27 (1H, m); | |
| | phenylacetyl]piperidin-4- | 7.63-7.52 (2H, m); | |
| | yl}pyrido[2,3-d]pyrimidine- | 7.38-7.33 (4H, m); 7.30-7.24 (2H, m); | |
| | 2,4 (1H,3H)-dione | 5.41 (1H, d); 4.99-4.88 (1H, | |
| | | m); 4.58-4.50 (1H, m); | |
| | | 4.10-4.02 (1H, m); 2.72-2.58 (1H, | |
| | | m); 2.42-2.29 (1H, m); | |
| | | 1.75-1.41 (2H, m) | |
| 78 | 1-(3,4-difluorophenyl)-6-fluoro- | (400 MHz, DMSO-d₆) δ | 527.1 |
| | 3-{1-[2-hydroxy-5- | 8.60 (1H, d); 8.32 (1H, dd); | |
| | (hydroxymethyl)benzoyl]piperidin- | 7.62-7.56 (2H, m); 7.30-7.27 (1H, | |
| | 4-yl}pyrido[2,3- | m); 7.14 (1H, d); 7.05 (1H, s); | |
| | d]pyrimidine-2,4-(1H,3H)-dione | 6.80 (1H, d); 5.05-4.99 (1H, | |
| | | m); 4.72-4.56 (1H, m); | |
| | | 4.37 (2H, s); 3.59-3.46 (1H, m); | |
| | | 3.14-3.01 (1H, m); | |
| | | 2.90-2.78 (1H, m); 1.82-1.59 (2H, m) | |

### EXAMPLE 79

### 1-(3,4-difluorophenyl)-6-fluoro-3-[(3R)-1-(1H-indazol-3-ylcarbonyl)pyrrolidin-3-yl]pyrido[2,3-d]pyrimidine-2,4(1H,3H)-dione

A mixture of 1-(3,4-difluorophenyl)-6-fluoro-3-[(3R)-pyrrolidin-3-yl]pyrido[2,3-d]pyrimidine-2,4(1H,3H)-dione hydrochloride (0.114 mmol), 1H-indazole-3-carboxylic acid (37 mg, 0.228 mmol), HATU (87 mg, 0.228 mmol), HOAT (31 mg, 0.228 mmol) and DIEA (98 μl, 0.570 mmol) in NMP (0.8 ml) was stirred for 2 h at room temperature. The title compound was obtained by purification on RP-HPLC (18 mg, 33%).

¹H NMR (400 MHz, DMSO-d₆): δ 8.61 (1H, dd); 8.38-8.33 (1H, m); 8.19-8.14 (1H, m); 7.65-7.54 (3H, m); 7.41 (1H, t); 7.32-7.28 (1H, m); 7.25-7.21 (1H, m); 5.74 (1H, q); 4.35 (2H, d); 4.10-3.92 (2H, m); 3.74-3.67 (1H, m); 2.61-2.41 (1H, m); 2.25-2.18 (1H, m).

APCI-MS m/z: 507.1 [MH⁺].

LC (method A) rt=10.3 min

### EXAMPLES 80 TO 82

The following compounds were synthesised in an analogous method to Example 52.

| Ex | Compound | ¹H NMR | m/z |
|---|---|---|---|
| 80 | 1-(3,4-difluorophenyl)-6- | (400 MHz, DMSO-d₆) δ 8.60 (1H, | 497.1 |
| | fluoro-3-{(3R)-1-[(2R)-2- | dd); 8.32 (1H, ddd); 7.64-7.22 (8H, | |
| | hydroxy-2- | m); 5.59-5.52 (1H, m); 5.24 (1H, d); | |
| | phenylacetyl]pyrrolidin-3- | 3.93-3.83 (2H, m); 3.63-3.56 (2H, | |
| | yl}pyrido[2,3- | m); 3.21-3.14 (1H, m); | |
| | d]pyrimidine-2,4(1H,3H)- | 2.43-2.34 (1H, m); 2.16-2.08 (1H, m). | |
| | dione | | |
| 81 | 3-{(3R)-1-[(2R)-2- | (400 MHz, DMSO-d₆) δ 8.61 (1H, t); | 503.1 |
| | cyclohexyl-2- | 8.37-8.32 (1H, m); 7.65-7.54 (2H, | |
| | hydroxyacetyl]pyrrolidin- | m); 7.29 (1H, d); 5.63 (1H, dt); | |
| | 3-yl}-1-(3,4- | 3.95-3.78 (3H, m); 3.67-3.46 (2H, m); | |
| | difluorophenyl)-6- | 2.47-2.40 (1H, m); 2.23-2.10 (1H, | |
| | fluoropyrido[2,3- | m); 1.65-1.48 (6H, m); | |
| | d]pyrimidine-2,4(1H,3H)- | 1.20-0.94 (5H, m). | |
| | dione | | |
| 82 | 1-(3,4-difluorophenyl)-6- | (400 MHz, DMSO-d₆) δ | 513.1 |
| | fluoro-3-{(3R)-1-[2- | 8.65-8.57 (1H, m); 8.38-8.31 (1H, m); | |
| | hydroxy-5- | 7.64-7.52 (2H, m); 7.31-7.24 (1H, m); | |
| | (hydroxymethyl)benzoyl]pyrrolidin- | 7.17 (2H, s); 6.82 (1H, s); 5.60 (1H, | |
| | 3-yl}pyrido[2,3- | d); 4.36 (2H, s); 3.86-3.53 (5H, m); | |
| | d]pyrimidine-2,4(1H,3H)- | 2.89 (1H, s); 2.63-2.41 (1H, m); | |
| | dione | 2.22-2.09 (1H, m). | |

### EXAMPLE 83

### 1-(3,4-difluorophenyl)-6-fluoro-3-[(3S)-1-(1H-indazol-3-ylcarbonyl)pyrrolidin-3-yl]pyrido[2,3-d]pyrimidine-2,4(1H,3H)-dione

A mixture of 1-(3,4-difluorophenyl)-6-fluoro-3-[(3S)-pyrrolidin-3-yl]pyrido[2,3-d]pyrimidine-2,4(1H,3H)-dione hydrochloride (0.17 mmol), 1H-indazole-3-carboxylic acid (56 mg, 0.35 mmol), HATU (133 mg, 0.35 mmol), HOAT (48 mg, 0.35 mmol) and DIEA (146 μl, 0.85 mmol) in NMP (1 ml) was stirred for 2 h at room temperature. The title compound was obtained by purification on RP-HPLC (12 mg, 14%).

¹H NMR (400 MHz, DMSO-d₆): δ 8.61 (1H, dd); 8.39-8.33 (1H, m); 8.19-8.14 (1H, m); 7.64-7.56 (3H, m); 7.41 (1H, t); 7.34-7.27 (1H, m); 7.26-7.20 (1H, m); 5.74 (1H, q); 4.35 (2H, d); 4.10-3.93 (2H, m); 3.75-3.67 (1H, m); 2.57-2.41 (1H, m); 2.26-2.17 (1H, m).

APCI-MS m/z: 507.1 [MH⁺].

LC (method A) rt=10.3 min

### EXAMPLE 84

The following compound was synthesised in an analogous method to Example 56.

| Ex | Compound | ¹H NMR | m/z |
|---|---|---|---|
| 84 | 1-(3,4-difluorophenyl)-6- | (400 MHZ, DMSO-d₆) δ 8.60 (1H, | 497.1 |
| | fluoro-3-{(3S)-1-[(2R)-2- | dd); 8.32 (1H, ddd); 7.64-7.22 | |
| | hydroxy-2- | m); (8H, 5.59-5.52 (1H, m); | |
| | phenylacetyl]pyrrolidin- | 5.24 (1H, d); 3.93-3.83 (2H, m); | |
| | 3-yl}pyrido[2,3- | 3.63-3.56 (2H, m); 3.21-3.14 (1H, | |
| | d]pyrimidine- | m); 2.43-2.34 (1H, m); | |
| | 2,4(1H,3H)-dione | 2.16-2.08 (1H, m). | |

Examples 85-107 were all prepared according to the procedure of Example 1.

| Ex | Compound | 1H NMR | m/z |
|---|---|---|---|
| 85 | N-{cis-4-[1-(3,4- | (500 MHz, DMSO-d₆): δ 11.25 (1H, | 534 |
| | difluorophenyl)-6- | s); 8.56 (1H, d); 8.30 (1H, dd); | |
| | fluoro-2,4-dioxo-1,4- | 8.13 (1H, s); 7.64 (1H, d); 7.61-7.55 (3H, | |
| | dihydropyrido[2,3- | m); 7.40-7.35 (2H, m); | |
| | d]pyrimidin-3(2H)- | 7.30-7.25 (1H, m); 6.45 (1H, s); 4.82 (1H, | |
| | yl]cyclohexyl}-1H- | d); 3.98 (1H, s); 2.77-2.64 (2H, | |
| | indole-5-carboxamide | m); 2.09 (2H, d); 1.63-1.46 (4H, m) | |
| 86 | N-{cis-4-[1-(3,4- | (500 MHz, DMSO-d₆): δ 8.66 (1H, | 540 |
| | difluorophenyl)-6- | t); 8.55 (1H, d); 8.36-8.32 (2H, | |
| | fluoro-2,4-dioxo-1,4- | m); 8.29-8.24 (2H, m); 7.74 (1H, | |
| | dihydropyrido[2,3- | t); 7.59-7.52 (2H, m); | |
| | d]pyrimidin-3(2H)- | 7.28-7.23 (1H, m); 4.84-4.76 (1H, m); | |
| | yl]cyclohexyl}-3- | 3.97 (1H, s); 2.74-2.61 (2H, m); 2.11 (2H, | |
| | nitrobenzamide | d); 1.64-1.47 (4H, m) | |
| 87 | N-{cis-4-[1-(3,4- | (500 MHz, DMSO-d₆): δ 11.54 (1H, | 534 |
| | difluorophenyl)-6- | s); 8.55 (1H, d); 8.29 (1H, dd); | |
| | fluoro-2,4-dioxo-1,4- | 7.81 (1H, d); 7.60-7.50 (3H, m); 7.39 (1H, | |
| | dihydropyrido[2,3- | d); 7.29-7.25 (1H, m); 7.22 (1H, | |
| | d]pyrimidin-3(2H)- | d); 7.16-7.12 (1H, m); 7.00 (1H, | |
| | yl]cyclohexyl}-1H- | dd); 4.80 (1H, ddd); 4.01 (1H, s); | |
| | indole-2-carboxamide | 2.73-2.60 (2H, m); 2.09 (2H, d); | |
| | | 1.67-1.48 (4H, m) | |
| 88 | N-{cis-4-[1-(3,4- | (500 MHz, DMSO-d₆): δ 9.07 (1H, | 535 |
| | difluorophenyl)-6- | s); 8.88 (1H, s); 8.56 (1H, d); 8.29 (1H, | |
| | fluoro-2,4-dioxo-1,4- | dd); 8.23 (1H, s); 8.07 (1H, d); | |
| | dihydropyrido[2,3- | 7.91 (1H, dd); 7.77-7.72 (1H, m); | |
| | d]pyrimidin-3(2H)- | 7.60-7.53 (2H, m); 7.29-7.23 (1H, | |
| | yl]cyclohexyl}-1H- | m); 4.86-4.73 (1H, m); 3.98 (1H, | |
| | benzimidazole-5- | s); 2.76-2.61 (2H, m); 2.11 (2H, | |
| | carboxamide | d); 1.65-1.46 (4H, m) | |
| 89 | 4-cyano-N-{cis-4-[1- | (500 MHz, DMSO-d₆): δ 8.55 (1H, | 520 |
| | (3,4-difluorophenyl)-6- | d); 8.29 (1H, dd); 8.23 (1H, d); | |
| | fluoro-2,4-dioxo-1,4- | 7.95 (4H, dd); 7.60-7.53 (2H, m); | |
| | dihydropyrido[2,3- | 7.29-7.23 (1H, m); 4.79 (1H, d); 3.94 (1H, | |
| | d]pyrimidin-3(2H)- | s); 2.73-2.62 (2H, m); | |
| | yl]cyclohexyl}benzamide | 2.11-2.04 (2H, m); 1.63-1.45 (4H, m) | |
| 90 | N-{cis-4-[1-(3,4- | (500 MHz, DMSO-d₆): δ 8.55 (1H, | 536 |
| | difluorophenyl)-6- | d); 8.48 (1H, s); 8.30 (1H, dd); | |
| | fluoro-2,4-dioxo-1,4- | 8.14 (1H, d); 7.90 (2H, s); 7.60-7.52 (2H, | |
| | dihydropyrido[2,3- | m); 7.27 (1H, d); 4.85-4.76 (1H, | |
| | d]pyrimidin-3(2H)- | m); 3.98 (1H, s); 2.73 (2H, d); | |
| | yl]cyclohexyl}-1H- | 2.12 (2H, d); 1.66-1.47 (4H, m) | |
| | 1,2,3-benzotriazole-5- | | |
| | carboxamide | | |
| 91 | N-{cis-4-[1-(3,4- | (500 MHz, DMSO-d₆): δ 9.49 (1H, | 552 |
| | difluorophenyl)-6- | s); 8.64 (1H, d); 8.56 (1H, d); | |
| | fluoro-2,4-dioxo-1,4- | 8.29 (1H, dd); 8.12 (1H, d); 8.07 (1H, | |
| | dihydropyrido[2,3- | d); 7.98 (1H, dd); 7.61-7.52 (2H, | |
| | d]pyrimidin-3(2H)- | m); 7.30-7.24 (1H, m); 4.82 (1H, | |
| | yl]cyclohexyl}-1,3- | d); 3.98 (1H, s); 2.76-2.65 (2H, | |
| | benzothiazole-6- | m); 2.11 (2H, d); 1.66-1.47 (4H, m) | |
| | carboxamide | | |
| 92 | N-{cis-4-[1-(3,4- | (500 MHz, DMSO-d₆): δ 10.37 (1H, | 571 |
| | difluorophenyl)-6- | s); 8.56 (1H, d); 8.23 (1H, s); 7.94 (1H, | |
| | fluoro-2,4-dioxo-1,4- | d); 7.61-7.52 (2H, m); | |
| | dihydropyrido[2,3- | 7.29-7.23 (1H, m); 4.83-4.73 (1H, m); | |
| | d]pyrimidin-3(2H)- | 3.99 (2H, t); 3.92-3.87 (1H, m); 3.47 (1H, | |
| | yl]cyclohexyl}-3- | s); *(5H in solvent peak); | |
| | methyl-6,7-dihydro-5H- | 2.09-1.96 (4H, m); 1.63-1.46 (4H, m) | |
| | [1,3]thiazolo[3,2- | | |
| | a]pyrimidine-2- | | |
| | carboxamide | | |
| 93 | N-{cis-4-[1-(3,4- | (500 MHz, DMSO-d₆): δ 9.65 (1H, | 503 |
| | difluorophenyl)-6- | s); 8.55 (1H, d); 8.30 (2H, dd); | |
| | fluoro-2,4-dioxo-1,4- | 7.55 (2H, d); 7.28-7.23 (1H, m); | |
| | dihydropyrido[2,3- | 4.85-4.76 (1H, m); 4.18-4.11 (1H, m); | |
| | d]pyrimidin-3(2H)- | *(2H in solvent peak); | |
| | yl]cyclohexyl}-1,2,3- | 2.08-2.00 (2H, m); 1.73-1.54 (4H, m) | |
| | thiadiazole-4- | | |
| | carboxamide | | |
| 94 | N-{cis-4-[1-(3,4- | (500 MHz, DMSO-d₆): δ 8.55 (1H, | 485 |
| | difluorophenyl)-6- | d); 8.28 (1H, dd); 8.06 (2H, s); | |
| | fluoro-2,4-dioxo-1,4- | 7.60-7.51 (2H, m); 7.40 (1H, d); | |
| | dihydropyrido[2,3- | 7.28-7.23 (1H, m); 4.81-4.70 (1H, m); | |
| | d]pyrimidin-3(2H)- | 3.89 (1H, s); *(2H in solvent peak); | |
| | yl]cyclohexyl}-1H- | 2.03 (2H, d); 1.61-1.43 (4H, m) | |
| | pyrazole-4- | | |
| | carboxamide | | |
| 95 | N-{cis-4-[1-(3,4- | (500 MHz, DMSO-d₆): δ 8.55 (1H, | 501 |
| | difluorophenyl)-6- | d); 8.28 (1H, dd); 8.17 (1H, dd); | |
| | fluoro-2,4-dioxo-1,4- | 7.66 (1H, d); 7.60-7.52 (3H, m); 7.50 (1H, | |
| | dihydropyrido[2,3- | dd); 7.29-7.22 (1H, m); | |
| | d]pyrimidin-3(2H)- | 4.82-4.73 (1H, m); 3.91 (1H, s); *(2H in solvent | |
| | yl]cyclohexyl}thiophene- | peak); 2.06 (2H, d); 1.60-1.42 (4H, | |
| | 3-carboxamide | m) | |
| 96 | N-{cis-4-[1-(3,4- | (500 MHz, DMSO-d₆): δ 11.06 (1H, | 484 |
| | difluorophenyl)-6- | s); 8.55 (1H, d); 8.28 (1H, dd); | |
| | fluoro-2,4-dioxo-1,4- | 7.60-7.51 (2H, m); 7.33 (1H, dt); | |
| | dihydropyrido[2,3- | 7.29-7.22 (1H, m); 6.98 (1H, d); 6.70 (1H, | |
| | d]pyrimidin-3(2H)- | q); 6.45 (1H, q); *(2H in solvent | |
| | yl]cyclohexyl}-1H- | peak); 2.00 (2H, d); 1.57-1.44 (4H, | |
| | pyrrole-3-carboxamide | m) | |
| 97 | N-{cis-4-[1-(3,4- | (500 MHz, DMSO-d₆): δ 8.55 (1H, | 499 |
| | difluorophenyl)-6- | d); 8.29 (1H, dd); 8.25-8.10 (1H, | |
| | fluoro-2,4-dioxo-1,4- | m); 7.87 (1H, s); 7.72 (1H, s); | |
| | dihydropyrido[2,3- | 7.61-7.51 (2H, m); 7.29-7.23 (1H, | |
| | d]pyrimidin-3(2H)- | m); 4.78 (1H, s); 4.04 (1H, t); | |
| | yl]cyclohexyl}-1- | 3.72 (3H, s); *(2H in solvent peak); | |
| | methyl-1H-imidazole- | 1.91 (2H, d); 1.69-1.49 (4H, m) | |
| | 4-carboxamide | | |
| 98 | N-{cis-4-[1-(3,4- | (500 MHz, DMSO-d₆): δ 8.55 (1H, | 485 |
| | difluorophenyl)-6- | d); 8.29 (1H, dd); 7.76 (1H, s); | |
| | fluoro-2,4-dioxo-1,4- | 7.63-7.50 (2H, m); 7.41 (1H, d); | |
| | dihydropyrido[2,3- | 7.29-7.22 (1H, m); 6.64 (1H, s); 4.79 (1H, | |
| | d]pyrimidin-3(2H)- | s); 4.08 *(2H in solvent peak); (1H, | |
| | yl]cyclohexyl}-1H- | d); 1.91 (2H, d); 1.68-1.50 (4H, m) | |
| | pyrazole-3- | | |
| | carboxamide | | |
| 99 | N-{cis-4-[1-(3,4- | (500 MHz, DMSO-d₆): δ | 516 |
| | difluorophenyl)-6- | 9.23-9.10 (1H, m); 8.56 (1H, d); 8.26 (1H, | |
| | fluoro-2,4-dioxo-1,4- | dd); 7.92 (1H, d); 7.60-7.52 (2H, | |
| | dihydropyrido[2,3- | m); 7.29-7.22 (1H, m); | |
| | d]pyrimidin-3(2H)- | 4.77-4.67 (1H, m); 3.73 (1H, s); 3.02-2.90 (3H, | |
| | yl]cyclohexyl}-1- | m); 2.88-2.77 (1H, m); | |
| | methylpiperidine-3- | 2.76-2.67 (3H, m); *(2H in solvent peak); | |
| | carboxamide | 1.99-1.28 (10H, m) | |
| 100 | N-{cis-4-[1-(3,4- | (500 MHz, DMSO-d₆): δ 8.56 (1H, | 532 |
| | difluorophenyl)-6- | d); 8.26 (1H, dd); 7.99-7.92 (1H, | |
| | fluoro-2,4-dioxo-1,4- | m); 7.61-7.51 (2H, m); | |
| | dihydropyrido[2,3- | 7.28-7.22 (1H, m); 4.79-4.69 (1H, m); | |
| | d]pyrimidin-3(2H)- | 3.95-3.87 (2H, m); 3.81-3.76 (1H, | |
| | yl]cyclohexyl}-3- | m); 3.64-3.54 (2H, m); | |
| | morpholin-4- | 3.08-2.96*(8H in solvent peak); (1H, | |
| | ylpropanamide | m); 1.90-1.81 (2H, m); | |
| | | 1.58-1.42 (4H, m) | |
| 101 | N-{cis-4-[1-(3,4- | (500 MHz, DMSO-d₆): δ 8.55 (1H, | 521 |
| | difluorophenyl)-6- | d); 8.29 (1H, dd); 7.88 (1H, d); | |
| | fluoro-2,4-dioxo-1,4- | 7.81 (2H, d); 7.61-7.49 (4H, m); | |
| | dihydropyrido[2,3- | 7.29-7.24 (1H, m); 6.75 (1H, dd); 5.91 (1H, | |
| | d]pyrimidin-3(2H)- | d); 5.33 (1H, d); 4.79 (1H, dd); | |
| | yl]cyclohexyl}-4- | 3.94 (1H, s); 2.73-2.60 (2H, m); | |
| | vinylbenzamide | 2.12-2.03 (2H, m); 1.63-1.44 (4H, m) | |
| 102 | N-{cis-4-[1-(3,4- | (500 MHz, DMSO-d₆): δ 8.55 (1H, | 519 |
| | difluorophenyl)-6- | d); 8.32-8.24 (1H, m); | |
| | fluoro-2,4-dioxo-1,4- | 8.04-7.98 (1H, m); 7.85-7.79 (2H, m); | |
| | dihydropyrido[2,3- | 7.61-7.49 (4H, m); 7.29-7.23 (1H, | |
| | d]pyrimidin-3(2H)- | m); 4.83-4.73 (1H, m); 4.31 (1H, | |
| | yl]cyclohexyl}-4- | s); 3.95-3.90 (1H, m); | |
| | ethynylbenzamide | 2.71-2.61*(2H in solvent peak); (2H, m); | |
| | | 2.11-2.03 (2H, m); 1.64-1.44 (4H, m) | |
| 103 | N-{cis-4-[1-(3,4- | (500 MHz, DMSO-d₆): δ 12.28 (1H, | 535 |
| | difluorophenyl)-6- | s); 8.55 (1H, d); 8.31 (1H, dd); | |
| | fluoro-2,4-dioxo-1,4- | 8.28 (1H, dd); 8.05 (1H, dd); 7.88 (1H, | |
| | dihydropyrido[2,3- | d); 7.59-7.51 (2H, m); | |
| | d]pyrimidin-3(2H)- | 7.28-7.24 (1H, m); 7.16 (1H, d); 7.10 (1H, | |
| | yl]cyclohexyl}-1H- | dd); 4.79 (1H, s); 4.01 (1H, s); | |
| | pyrrolo[2,3-b]pyridine- | 2.73-2.60 (2H, m); 2.10-2.04 (2H, | |
| | 2-carboxamide | m); 1.68-1.48 (4H, m) | |
| 104 | N-{cis-4-[1-(3,4- | (500 MHz, DMSO-d₆): δ 8.55 (1H, | 552 |
| | difluorophenyl)-6- | d); 8.29 (1H, dd); 8.27 (1H, d); | |
| | fluoro-2,4-dioxo-1,4- | 8.19 (1H, d); 8.14 (1H, d); 7.62-7.52 (4H, | |
| | dihydropyrido[2,3- | m); 7.29-7.25 (1H, m); | |
| | d]pyrimidin-3(2H)- | 4.88-4.76 (1H, m); 4.12-4.04 *(2H in solvent | |
| | yl]cyclohexyl}-1,3- | peak), (1H, m); 2.14-2.03 (2H, | |
| | benzothiazole-2- | m); 1.73-1.54 (4H, m) | |
| | carboxamide | | |
| 105 | N-{cis-4-[1-(3,4- | (500 MHz, DMSO-d₆): δ 8.61 (1H, | 552 |
| | difluorophenyl)-6- | dd); 8.55 (1H, d); 8.34 (1H, dd); | |
| | fluoro-2,4-dioxo-1,4- | 8.32-8.26 (2H, m); 8.23 (1H, s); | |
| | dihydropyrido[2,3- | 7.59-7.52 (2H, m); 7.46 (1H, dd); | |
| | d]pyrimidin-3(2H)- | 7.29-7.24 (1H, m); 4.84-4.74 (1H, m); | |
| | yl]cyclohexyl}thieno[2, | 3.96 (1H, s); 2.75-2.60 (2H, m); | |
| | 3-b]pyridine-2- | 2.15-2.05 (2H, m); 1.66-1.46 (4H, m) | |
| | carboxamide | | |
| 106 | N-{cis-4-[1-(3,4- | (500 MHz, DMSO-d₆): δ 8.80 (1H, | 553 |
| | difluorophenyl)-6- | d); 8.67 (1H, d); 8.55 (1H, d); | |
| | fluoro-2,4-dioxo-1,4- | 8.50 (1H, s); 8.47 (1H, d); 8.29 (1H, | |
| | dihydropyrido[2,3- | dd); 7.58-7.50 (2H, m); | |
| | d]pyrimidin-3(2H)- | 7.28-7.24 (1H, m); 4.86-4.73 (1H, m); | |
| | yl]cyclohexyl}thieno[2, | 4.00-3.93 (1H, m); 2.71 (2H, d); 2.12 (2H, | |
| | 3-b]pyrazine-6- | d); 1.68-1.48 (4H, m) | |
| | carboxamide | | |
| 107 | N-{cis-4-[1-(3,4- | (500 MHz, DMSO-d₆): δ 9.44 (1H, | 537 |
| | difluorophenyl)-6- | dd); 8.96 (1H, dd); 8.55 (1H, d); | |
| | fluoro-2,4-dioxo-1,4- | 8.30 (1H, dd); 8.06 (1H, d); | |
| | dihydropyrido[2,3- | 7.60-7.51 (2H, m); 7.44 (1H, dd); | |
| | d]pyrimidin-3(2H)- | 7.32-7.22 (1H, m); 4.86-4.74 (1H, m); | |
| | yl]cyclohexyl}[1,2,4]triazolo | 4.18-4.10 (1H, m); *(2H in solvent peak); | |
| | [1,5-a]pyrimidine- | 2.06-1.93 (2H, m); 1.76-1.52 (4H, | |
| | 2-carboxamide | m) | |

### EXAMPLE 108

### Human Phosphodiesterase B2 Alpha Screen Assay

The assay uses recombinant Human Phosphodiesterase B2 (PDE4B2) produced in house (PrAZL0133), stored at −20° C. The substrate uses cAMP, part of the Alpha Screen cAMP kit (Perkin Elmer, Cat# 6760625M), stored at 4° C. The Alpha Screen kit also includes biotinylated cAMP, acceptor and donor beads.

The assay additions were as follows: Test compounds and controls were added to white 384-well flat-bottom plates (Greiner, Cat# 781075), 0.2 μl in 100% DMSO, followed by 10 μl PDE4B2 in reaction buffer. The reaction buffer constitution was: 50 mM Tris (pH 7.5), 8.3 mM MgCl2, 1.7 mM EGTA and 0.01% (w/v) Brij®35. The enzyme and the compounds were incubated at room temperature for 15 minutes. Then 10 μl cAMP in reaction buffer was added. The assay was stopped after 60 minutes incubation at room temperature by adding 10 μl acceptor beads in detection buffer with 40 mM EDTA. The detection buffer constitution was: 5 mM Tris (pH 7.5), 0.1% (w/v) BSA and 0.1% (v/v) Tween 20. This addition followed by an addition of 10 μl donor beads in detection buffer, with biotinylated cAMP. The plates were then incubated, dark at room temperature, for 5 hours followed by measurement on a Fusion™-α analyser. IC₅₀ values (presented in Table 1) were determined using Xlfit curve fitting using model 205.

TABLE 1

| Ex | PDE4B2 IC50 (nM) |
|---|---|
| 1 | 4.9 |
| 2 | 0.9 |
| 3 | 16.3 |
| 4 | 5.0 |
| 5 | 2.2 |
| 6 | 2.3 |
| 7 | 3.0 |
| 8 | 2.2 |
| 9 | 1.3 |
| 10 | 2.6 |
| 11 | 3.4 |
| 12 | 2.2 |
| 13 | 3.2 |
| 14 | 1.5 |
| 15 | 1.3 |
| 16 | 2.1 |
| 17 | 1.8 |
| 18 | 2.7 |
| 19 | 2.0 |
| 20 | 3.6 |
| 21 | 3.6 |
| 22 | 1.0 |
| 23 | 3.8 |
| 24 | 0.6 |
| 25 | 1.9 |
| 26 | 1.0 |
| 27 | 1.7 |
| 28 | 0.9 |
| 29 | 1.2 |
| 30 | 1.1 |
| 31 | 1.5 |
| 32 | 0.6 |
| 33 | 0.5 |
| 34 | 3.2 |
| 35 | 1.4 |
| 36 | 2.9 |
| 37 | 0.5 |
| 38 | <16 |
| 39 | 23.7 |
| 40 | 90.6 |
| 41 | 51.9 |
| 42 | <16 |
| 43 | 36.4 |
| 44 | 432.0 |
| 45 | <16 |
| 46 | 129.0 |
| 47 | 60.1 |
| 48 | 8.8 |
| 49 | 14.5 |
| 50 | 2.1 |
| 51 | 5.2 |
| 52 | 18.6 |
| 53 | 0.4 |
| 54 | 3.9 |
| 55 | 2.6 |
| 56 | 7.2 |
| 57 | 4.2 |
| 58 | 3.9 |
| 59 | 14.9 |
| 60 | 32.4 |
| 61 | 5.2 |
| 62 | 6.3 |
| 63 | 5.7 |
| 64 | 4.4 |
| 65 | 11.1 |
| 66 | 4.8 |
| 67 | 21.2 |
| 68 | 0.6 |
| 69 | 1.0 |
| 70 | 6.6 |
| 71 | 41.2 |
| 72 | 2.3 |
| 73 | 62.6 |
| 74 | 5.1 |
| 75 | 2.6 |
| 76 | 9.5 |
| 77 | 147.0 |
| 78 | 293.0 |
| 79 | 15.4 |
| 80 | 73.3 |
| 81 | 8.1 |
| 82 | 71.8 |
| 83 | 9.2 |
| 84 | 57.3 |
| 85 | 0.996 |
| 86 | 4.13 |
| 87 | 0.885 |
| 88 | 3.38 |
| 89 | 10.8 |
| 90 | 6.11 |
| 91 | 2.09 |
| 92 | 7.83 |
| 93 | 11.7 |
| 94 | 5.31 |
| 95 | 3.39 |
| 96 | 6.68 |
| 97 | 2.18 |
| 98 | 50.1 |
| 99 | 6.01 |
| 100 | 2.12 |
| 101 | 2.05 |
| 102 | 1 |
| 103 | 0.741 |
| 104 | 2.09 |
| 105 | 6.03 |
| 106 | 3.89 |
| 107 | 30.1 |

1. A compound of formula (I): 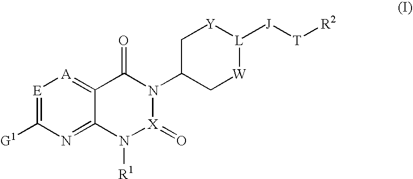
wherein:
A is N or CA¹;
E is N or CE¹;
T is C(O) or S(O)₂;
X is C or S;
W is (CH₂)ₙ;
Y is (CH₂)ₚ;
n and p are, independently 0 or 1;
L is CH or N;
when L is CH then J is NH; and when L is N then J is absent and T is bonded directly to L;
R¹ is aryl or heteroaryl, each of which is optionally substituted by halogen, cyano, hydroxy, C₁₋₄ alkyl, C₁₋₄ alkoxy, CF₃, OCF₃, C₁₋₄ alkylthio, S(O)(C₁₋₄ alkyl), S(O)₂(C₁₋₄ alkyl) or C(O)₂(C₁₋₄ alkyl);
R² is C₁₋₆ alkyl (optionally substituted by hydroxyl, C₁₋₆ alkoxy, heterocyclyl (optionally substituted by C₁₋₆ alkyl), aryl, heteroaryl, C₃₋₇ cycloalkyl, CO₂H, CO₂(C₁₋₆ alkyl) or NHC(O)R³), C 6 alkoxy, C₃₋₆ cycloalkyl (optionally substituted by hydroxyl or C₁₋₆ alkyl), heterocyclyl (optionally substituted by C₁₋₆ alkyl), aryl or heteroaryl;
R³ is C₁₋₆ alkyl or phenyl;
the foregoing phenyl, aryl and heteroaryl moieties of R² and R³ are, independently, optionally substituted by: halogen, cyano, nitro, hydroxy, S(O)_{q}R⁴, OC(O)NR⁵R⁶, NR⁷R⁸, NR⁹C(O)R¹⁰, NR¹¹C(O)NR¹²R¹³, S(O)₂NR¹⁴R¹⁵, NR¹⁶S(O)₂R¹⁷, C(O)NR¹⁸R¹⁹, C(O)R²⁰, CO₂R²¹, NR²²CO₂R²³, C₁₋₆ alkyl, C₁₋₆ hydroxyalkyl, C₁₋₆ haloalkyl, C₁₋₆ alkoxy(C₁₋₆)alkyl, di(C₁₋₆)alkylamino(C₁₋₆)alkyl, C₁₋₆ alkoxy, C₁₋₆ haloalkoxy, C₁₋₆ alkoxy(C₁₋₆)alkoxy, C₁₋₆ alkylthio, C₂₋₆ alkenyl, C₂₋₆ alkynyl, C₃₋₁₀ cycloalkyl (itself optionally substituted by C₁₋₄ alkyl or oxo), methylenedioxy, difluoromethylenedioxy, phenyl, phenyl(C₁₋₄)alkyl, phenoxy, phenylthio, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halogen, hydroxy, nitro, S(O)ᵣ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
A¹, E¹ and G¹ are, independently, hydrogen, halogen, cyano, hydroxy, C₁₋₄ alkyl, C₁₋₄ alkoxy, CF₃ or OCF₃;
q and r are, independently, 0, 1 or 2;
R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are, independently, C₁₋₆ alkyl {optionally substituted by halogen, hydroxy or C₁₋₆ alkoxy}, CH₂(C₂₋₆ alkenyl), phenyl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃} or heteroaryl {itself optionally substituted by halogen, hydroxy, nitro, NH₂, NH(C₁₋₄ alkyl), N(C₁₋₄ alkyl)₂, S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃};
R⁵, R⁶, R⁷, R⁸, R⁹, R¹⁰, R¹¹, R¹², R¹³, R¹⁴, R¹⁵, R¹⁶, R¹⁸, R¹⁹, R²⁰, R²¹, R²² and R²³ can also be hydrogen;
or a N-oxide thereof; or a pharmaceutically acceptable salt thereof.

2. A compound of formula (I) as claimed in claim 1 wherein A is CA¹; E is CE¹; and A¹, E¹ and G¹ are, independently, hydrogen or halogen.

3. A compound of formula (I) as claimed in claim 1 wherein X is C.

4. A compound of formula (I) as claimed in claim 1, wherein n and p are both 1.

5. A compound of formula (I) as claimed in claim 1, wherein L is CH.

6. A compound of formula (I) as claimed in claim 1 wherein J is NH.

7. A compound of formula (J) as claimed in claim 1 wherein T is C(O).

8. A compound of formula (I) as claimed in claim 1 wherein R¹ is aryl optionally substituted by halogen, C₁₋₄ alkylthio, S(O)₂(C₁₋₄ alkyl) or C(O)₂(C₁₋₄ alkyl).

9. A compound of formula (I) as claimed in claim 1 wherein R² is C₁₋₆ alkyl {optionally substituted by hydroxy, phenyl (itself optionally substituted by hydroxyl), C₃₋₇ cycloalkyl, CO₂(C₁₋₄ alkyl) or NHC(O)R³}, phenyl {optionally substituted by halogen, hydroxyl, C₁₋₄ alkyl, C₁₋₄ alkoxy, CO₂(C₁₋₄ alkyl) or C₂₋₄ alkynyl} or heteroaryl {optionally substituted by halogen, hydroxyl, C₁₋₄ alkyl, C₁₋₄ alkoxy, CO₂(C₁₋₄ alkyl) or C₂₋₄ alkynyl}; wherein R³ is phenyl optionally substituted by hydroxyl.

10. A compound of formula (I) as claimed in claim 1 that is:
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-5-(hydroxymethyl)benzamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-2-phenylacetamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxyacetamide;
5-chloro-N-{cis-4-[1-(3,4-difluorophenyl)-6-fluoro-2,4-dioxo-1,4-dihydropyrido[2,3-d]pyrimidin-3 (2H)-yl]cyclohexyl}-2-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-4-hydroxybenzamide;
methyl 2-[({cis-4-[1-(3,4-difluorophenyl)-6-fluoro-2,4-dioxo-1,4-dihydropyrido[2,3-d]pyrimidin-3(2H)-yl]cyclohexyl}amino)carbonyl]benzoate;
N-{cis-4-[1-(3,4-difluorophenyl)-6-fluoro-2,4-dioxo-1,4-dihydropyrido[2,3-d]pyrimidin-3(2H)-yl]cyclohexyl}-2-(4-hydroxyphenyl)acetamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-4-methylbenzamide;
N-{cis-4-[1-(3,4-difluorophenyl)-6-fluoro-2,4-dioxo-1,4-dihydropyrido[2,3-d]pyrimidin-3 (2H)-yl]cyclohexyl}-2-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-hydroxypyridine-2-carboxamide;
3-chloro-N-{cis-4-[1-(3,4-difluorophenyl)-6-fluoro-2,4-dioxo-1,4-dihydropyrido[2,3-d]pyrimidin-3(2H)-yl]cyclohexyl}-4-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-hydroxy-2-methylbenzamide;
N-{cis-4-[1-(3,4-difluorophenyl)-6-fluoro-2,4-dioxo-1,4-dihydropyrido[2,3-d]pyrimidin-3 (2H)-yl]cyclohexyl}-4-hydroxy-3-methoxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-hydroxy-4-methylbenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-6-methoxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-3-methoxybenzamide;
4-chloro-N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-4-methoxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-5-fluoro-2-hydroxybenzamide;
(2S)—N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-4-methylpentanamide;
(2R)-2-cyclohexyl-N-{cis-4-[1-(3,4-difluorophenyl)-6-fluoro-2,4-dioxo-1,4-dihydropyrido[2,3-d pyrimidin-3(2H)-yl]cyclohexyl}-2-hydroxyacetamide;
methyl 4-({cis-4-[1-(3,4-difluorophenyl)-6-fluoro-2,4-dioxo-1,4-dihydropyrido[2,3-d]pyrimidin-3(2H)-yl]cyclohexyl}amino)-4-oxobutanoate;
N-[2-({cis-4-[1-(3,4-difluorophenyl)-6-fluoro-2,4-dioxo-1,4-dihydropyrido[2,3-d]pyrimidin-3 (2H)-yl]cyclohexyl}amino)-2-oxoethyl]-2-hydroxybenzamide;
methyl 4-[({cis-4-[1-(3,4-difluorophenyl)-6-fluoro-2,4-dioxo-1,4-dihydropyrido[2,3-d]pyrimidin-3(2H)-yl]cyclohexyl}amino)carbonyl]benzoate;
N-{cis-4-[1-(3,4-difluorophenyl)-6-fluoro-2,4-dioxo-1,4-dihydropyrido[2,3-d]pyrimidin-3(2H)-yl]cyclohexyl}-3-hydroxy-4-methoxybenzamide;
methyl 5-({cis-4-[1-(3,4-difluorophenyl)-6-fluoro-2,4-dioxo-1,4-dihydropyrido[2,3-d]pyrimidin-3(2H)-yl]cyclohexyl}amino)-5-oxopentanoate;
N-{cis-4-[1-(3,4-difluorophenyl)-6-fluoro-2,4-dioxo-1,4-dihydropyrido[2,3-d]pyrimidin-3(2H)-yl]cyclohexyl}-2-(3-hydroxyphenyl)acetamide;
methyl 3-[({cis-4-[1-(3,4-difluorophenyl)-6-fluoro-2,4-dioxo-1,4-dihydropyrido[2,3-d]pyrimidin-3(2H)-yl]cyclohexyl}amino)carbonyl]benzoate;
N-{cis-4-[1-(3,4-difluorophenyl)-6-fluoro-2,4-dioxo-1,4-dihydropyrido[2,3-d]pyrimidin-3(2H)-yl]cyclohexyl}-2-fluoro-6-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-(3-hydroxyphenyl)propanamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-indazole-3-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}imidazo[1,2-a]pyridine-2-carboxamide;
5-chloro-N-{cis-4-[6-fluoro-1-[3-(methylthio)phenyl]-2,4-dioxo-1,4-dihydropyrido[2,3-d]pyrimidin-3 (2H)-yl]cyclohexyl}-2-hydroxybenzamide;
N-{cis-4-[6-fluoro-1-[3-(methylthio)phenyl]-2,4-dioxo-1,4-dihydropyrido[2,3-d]pyrimidin-3 (2H)-yl]cyclohexyl}-2-hydroxy-5-methylbenzamide;
N-{cis-4-[6-fluoro-1-[3-(methylthio)phenyl]-2,4-dioxo-1,4-dihydropyrido[2,3-d]pyrimidin-3(2H)-yl]cyclohexyl}-2-hydroxy-5-(hydroxymethyl)benzamide;
(2R)—N-{cis-4-[6-fluoro-1-[3-(methylthio)phenyl]-2,4-dioxo-1,4-dihydropyrido[2,3-d]pyrimidin-3(2H)-yl]cyclohexyl}-2-hydroxy-2-phenylacetamide;
(2S)—N-{cis-4-[6-fluoro-1-[3-(methylthio)phenyl]-2,4-dioxo-1,4-dihydropyrido[2,3-d]pyrimidin-3(2H)-yl]cyclohexyl}-2-hydroxy-2-phenylacetamide;
N-{cis-4-[6-fluoro-1-[3-(methylthio)phenyl]-2,4-dioxo-1,4-dihydropyrido[2,3-d]pyrimidin-3(2H)-yl]cyclohexyl}-2-hydroxyacetamide;
5-chloro-N-{cis-4-[6-fluoro-1-[3-(methylsulfonyl)phenyl]-2,4-dioxo-1,4-dihydropyrido[2,3-d]pyrimidin-3(2H)-yl]cyclohexyl}-2-hydroxybenzamide;
N-{cis-4-[6-fluoro-1-[3-(methylsulfonyl)phenyl]-2,4-dioxo-1,4-dihydropyrido[2,3-d]pyrimidin-3(2H)-yl]cyclohexyl}-2-hydroxy-5-(hydroxymethyl)benzamide;
(2R)—N-{cis-4-[6-fluoro-1-[3-(methylsulfonyl)phenyl]-2,4-dioxo-1,4-dihydropyrido[2,3-d]pyrimidin-3(2H)-yl]cyclohexyl}-2-hydroxy-2-phenylacetamide;
(2S)—N-{cis-4-[6-fluoro-1-[3-(methylsulfonyl)phenyl]-2,4-dioxo-1,4-dihydropyrido[2,3-d]pyrimidin-3(2H)-yl]cyclohexyl}-2-hydroxy-2-phenylacetamide;
N-{cis-4-[6-fluoro-1-[3-(methylsulfonyl)phenyl]-2,4-dioxo-1,4-dihydropyrido[2,3-d]pyrimidin-3(2H)-yl]cyclohexyl}-2-hydroxyacetamide;
methyl 3-[6-fluoro-3-{cis-4-[(2-hydroxy-5-methylbenzoyl)amino]cyclohexyl}-2,4-dioxo-3,4-dihydropyrido[2,3-d]pyrimidin-1(2H)-yl]benzoate;
N-{cis-4-[1-(3,4-difluorophenyl)-6-fluoro-2,4-dioxo-1,4-dihydropyrido[2,3-d]pyrimidin-3(2H)-yl]cyclohexyl}methanesulfonamide;
N-{cis-4-[1-(3,4-difluorophenyl)-6-fluoro-2-oxido-4-oxo-1,4-dihydro-3H-pyrido[2,3-c][1,2,6]thiadiazin-3-yl]cyclohexyl}-2-hydroxy-5-methylbenzamide;
N-{cis-4-[1-(3,4-Difluorophenyl)-6-fluoro-2,4-dioxo-1,4-dihydropyrido[2,3-d]pyrimidin-3(2H)-yl]cyclohexyl}cyclopropanecarboxamide;
Methyl 3-[6-fluoro-3-{cis-4-[(1-methyl-L-prolyl)amino]cyclohexyl}-2,4-dioxo-3,4-dihydropyrido[2,3-d]pyrimidin-1(2H)-yl]benzoate;
methyl 5-[({cis-4-[1-(3,4-difluorophenyl)-6-fluoro-2,4-dioxo-1,4-dihydropyrido[2,3-d]pyrimidin-3(2H)-yl]cyclohexyl}amino)carbonyl]pyridine-2-carboxylate;
N-{cis-4-[1-(3,4-difluorophenyl)-6-fluoro-2,4-dioxo-1,4-dihydropyrido[2,3-d]pyrimidin-3 (2H)-yl]cyclohexyl}-4-fluoro-2-hydroxybenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-1-hydroxycyclopropanecarboxamide;
N-{cis-4-[1-(3,4-difluorophenyl)-6-fluoro-2,4-dioxo-1,4-dihydropyrido[2,3-d]pyrimidin-3(2H)-yl]cyclohexyl}quinoline-8-carboxamide;
N-{cis-4-[6-fluoro-1-[3-(methylthio)phenyl]-2,4-dioxo-1,4-dihydropyrido[2,3-d]pyrimidin-3(2H)-yl]cyclohexyl}-2-furamide;
N-{cis-4-[6-fluoro-1-[3-(methylthio)phenyl]-2,4-dioxo-1,4-dihydropyrido[2,3-d]pyrimidin-3 (2H)-yl]cyclohexyl}cyclopropanecarboxamide;
N-{cis-4-[6-fluoro-1-[3-(methylthio)phenyl]-2,4-dioxo-1,4-dihydropyrido[2,3-d]pyrimidin-3(2H)-yl]cyclohexyl}propanamide;
N-{cis-4-[6-fluoro-1-[3-(methylthio)phenyl]-2,4-dioxo-1,4-dihydropyrido[2,3-d]pyrimidin-3 (2H)-yl]cyclohexyl}benzamide;
N-{cis-4-[6-fluoro-1-[3-(methylthio)phenyl]-2,4-dioxo-1,4-dihydropyrido[2,3-d]pyrimidin-3 (2H)-yl]cyclohexyl}cyclopentanecarboxamide;
N-{cis-4-[6-fluoro-1-[3-(methylthio)phenyl]-2,4-dioxo-1,4-dihydropyrido[2,3-d]pyrimidin-3(2H)-yl]cyclohexyl}-2-hydroxy-2-methylpropanamide;
N-{cis-4-[6-fluoro-1-[3-(methylthio)phenyl]-2,4-dioxo-1,4-dihydropyrido[2,3-d]pyrimidin-3(2H)-yl]cyclohexyl}benzenesulfonamide;
methyl 3-[3-{cis-4-[(cyclobutylcarbonyl)amino]cyclohexyl}-6-fluoro-2,4-dioxo-3,4-dihydropyrido[2,3-d]pyrimidin-1(2H)-yl]benzoate;
methyl 3-[6-fluoro-3-[cis-4-(isobutyrylamino)cyclohexyl]-2,4-dioxo-3,4-dihydropyrido[2,3-d]pyrimidin-1(2H)-yl]benzoate;
methyl 3-[6-fluoro-3-{cis-4-[(3-methylbutanoyl)amino]cyclohexyl}-2,4-dioxo-3,4-dihydropyrido[2,3-d]pyrimidin-1(2H)-yl]benzoate;
methyl 3-[3-{cis-4-[(2,2-dimethylpropanoyl)amino]cyclohexyl}-6-fluoro-2,4-dioxo-3,4-dihydropyrido[2,3-d]pyrimidin-1(2H)-yl]benzoate;
methyl 3-[6-fluoro-3-{cis-4-[(methoxyacetyl)amino]cyclohexyl}-2,4-dioxo-3,4-dihydropyrido[2,3-d]pyrimidin-1(2H)-yl]benzoate;
methyl 3-[6-fluoro-2,4-dioxo-3-{cis-4-[(2-thienylcarbonyl)amino]cyclohexyl}-3,4-dihydropyrido[2,3-d]pyrimidin-1(2H)-yl]benzoate;
methyl 3-[3-[cis-4-(acetylamino)cyclohexyl]-6-fluoro-2,4-dioxo-3,4-dihydropyrido[2,3-d]pyrimidin-1(2H)-yl]benzoate;
methyl 3-[6-fluoro-2,4-dioxo-3-{cis-4-[(pyrazolo[1,5-a]pyridin-2-ylcarbonyl)amino]cyclohexyl}-3,4-dihydropyrido[2,3-d]pyrimidin-1(2H)-yl]benzoate;
methyl 3-[6-fluoro-3-{cis-4-[(imidazo[1,2-a]pyridin-2-ylcarbonyl)amino]-cyclohexyl}-2,4-dioxo-3,4-dihydropyrido[2,3-d]pyrimidin-1(2H)-yl]benzoate;
methyl 3-[3-{cis-4-[(cyclopropylcarbonyl)amino]cyclohexyl}-6-fluoro-2,4-dioxo-3,4-dihydropyrido[2,3-d]pyrimidin-1(2H)-yl]benzoate;
N-{cis-4-[6-fluoro-1-[3-(methylsulfonyl)phenyl]-2,4-dioxo-1,4-dihydropyrido[2,3-d]pyrimidin-3(2H)-yl]cyclohexyl}cyclopropanecarboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-2-hydroxy-5-methylbenzamide;
methyl 3-[6-fluoro-3-(cis-4-{[(1-methylpiperidin-4-yl)carbonyl]amino}cyclohexyl)-2,4-dioxo-3,4-dihydropyrido[2,3-d]pyrimidin-1(2H)-yl]benzoate;
methyl 3-[3-[cis-4-({5-[(dimethylamino)methyl]-2-furoyl}amino)cyclohexyl]-6-fluoro-2,4-dioxo-3,4-dihydropyrido[2,3-d]pyrimidin-1(2H)-yl]benzoate;
methyl 3-[3-{cis-4-[(cyclohexylcarbonyl)amino]cyclohexyl}-6-fluoro-2,4-dioxo-3,4-dihydropyrido[2,3-d]pyrimidin-1(2H)-yl]benzoate;
1-(3,4-difluorophenyl)-6-fluoro-3-[1-(1H-indazol-3-ylcarbonyl)piperidin-4-yl]pyrido[2,3-d]pyrimidine-2,4(1H,3H)-dione;
1-(3,4-difluorophenyl)-6-fluoro-3-{1-[(2R)-2-hydroxy-2-phenylacetyl]piperidin-4-yl}pyrido[2,3-d]pyrimidine-2,4(1H,3H)-dione;
1-(3,4-difluorophenyl)-6-fluoro-3-{1-[2-hydroxy-5-(hydroxymethyl)benzoyl]piperidin-4-yl}pyrido[2,3-d]pyrimidine-2,4(1H,3H)-dione;
1-(3,4-difluorophenyl)-6-fluoro-3-[(3R)-1-(1H-indazol-3-ylcarbonyl)pyrrolidin-3-yl]pyrido[2,3-d]pyrimidine-2,4(1H,3H)-dione;
1-(3,4-difluorophenyl)-6-fluoro-3-{(3R)-1-[(2R)-2-hydroxy-2-phenylacetyl]pyrrolidin-3-yl}pyrido[2,3-d]pyrimidine-2,4(1H,3H)-dione;
3-{(3R)-1-[(2R)-2-cyclohexyl-2-hydroxyacetyl]pyrrolidin-3-yl}-1-(3,4-difluorophenyl)-6-fluoropyrido[2,3-d]pyrimidine-2,4(1H,3H)-dione;
1-(3,4-difluorophenyl)-6-fluoro-3-{(3R)-1-[2-hydroxy-5-(hydroxymethyl)benzoyl]pyrrolidin-3-yl}pyrido[2,3-d]pyrimidine-2,4(1H,3H)-dione;
1-(3,4-difluorophenyl)-6-fluoro-3-[(3S)-1-(1H-indazol-3-ylcarbonyl)pyrrolidin-3-yl]pyrido[2,3-d]pyrimidine-2,4(1H,3H)-dione;
1-(3,4-difluorophenyl)-6-fluoro-3-{(3S)-1-[(2R)-2-hydroxy-2-phenylacetyl]pyrrolidin-3-yl}pyrido[2,3-d]pyrimidine-2,4(1H,3H)-dione;
N-{cis-4-[1-(3,4-difluorophenyl)-6-fluoro-2,4-dioxo-1,4-dihydropyrido[2,3-d]pyrimidin-3(2H)-yl]cyclohexyl}-1H-indole-5-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-nitrobenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-indole-2-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-benzimidazole-5-carboxamide;
4-cyano-N-{cis-4-[1-(3,4-difluorophenyl)-6-fluoro-2,4-dioxo-1,4-dihydropyrido[2,3-d]pyrimidin-3(2H)-yl]cyclohexyl}benzamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-1,2,3-benzotriazole-5-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1,3-benzothiazole-6-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-methyl-6,7-dihydro-5H-[1,3]thiazolo[3,2-a]pyrimidine-2-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1,2,3-thiadiazole-4-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-pyrazole-4-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}thiophene-3-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-pyrrole-3-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1-methyl-1H-imidazole-4-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-pyrazole-3-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1-methylpiperidine-3-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-3-morpholin-4-ylpropanamide;
N-{cis-4-[1-(3,4-difluorophenyl)-6-fluoro-2,4-dioxo-1,4-dihydropyrido[2,3-d]pyrimidin-3(2H)-yl]cyclohexyl}-4-vinylbenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-4-ethynylbenzamide;
N-{cis-4-[1-(3,4-difluorophenyl)-6-fluoro-2,4-dioxo-1,4-dihydropyrido[2,3-d]pyrimidin-3(2H)-yl]cyclohexyl}-1H-pyrrolo[2,3-b]pyridine-2-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}-1,3-benzothiazole-2-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}thieno[2,3-b]pyridine-2-carboxamide;
N-{cis-4-[1-(3,4-difluorophenyl)-6-fluoro-2,4-dioxo-1,4-dihydropyrido[2,3-d]pyrimidin-3(2H)-yl]cyclohexyl}thieno[2,3-b]pyrazine-6-carboxamide; or,
N-{cis-4-[1-(3,4-difluorophenyl)-6-fluoro-2,4-dioxo-1,4-dihydropyrido[2,3-d]pyrimidin-3(2H)-yl]cyclohexyl}[1,2,4]triazolo[1,5-a]pyrimidine-2-carboxamide;
or a pharmaceutically acceptable salt thereof.

11. A process for the preparation of a compound of formula (I) comprising:
a. when X is C, which comprises removing the Boc protecting group from a compound of formula (II): 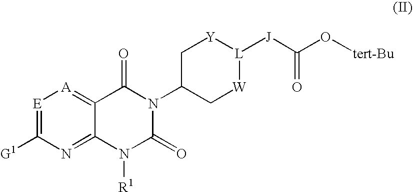
wherein R¹, G¹, E, A, Y, L, W, and J are as defined in claim 1; and then reacting the product so formed with an acid or acid derivative of formula (III): 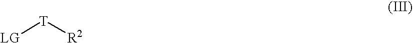
wherein R² and T are as defined in claim 1, and LG is a leaving group; at a suitable temperature and in a suitable solvent; or,
b. when X is S, by reacting a compound of formula (IIa): 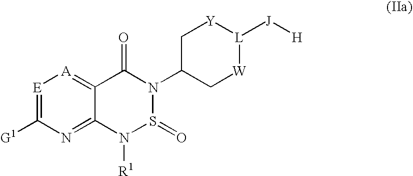
wherein R¹, G¹, E, A, Y, L, W, and J are as defined in claim 1, with an acid or acid derivative of formula (III): 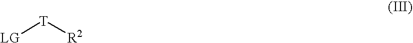
wherein R² and T are as defined in claim 1, and LG is a leaving group; at a suitable temperature and in a suitable solvent.

12. A pharmaceutical composition which comprises a compound of the formula (I), or a pharmaceutically acceptable salt thereof as claimed in claim 1, and a pharmaceutically acceptable adjuvant, diluent or carrier.

13. A combination comprising a compound of formula (I) and one or more agents selected from the list comprising:
a non-steroidal glucocorticoid receptor (GR-receptor) agonist;
a selective β.sub2. adrenoceptor agonist such as metaproterenol, isoproterenol, isoprenaline, albuterol, salbutamol, formoterol, salmeterol, terbutaline, orciprenaline, bitolterol mesylate, pirbuterol or indacaterol;
a muscarinic receptor antagonist (for example a M1, M2 or M3 antagonist, such as a selective M3 antagonist) such as ipratropium bromide, tiotropium bromide, oxitropium bromide, pirenzepine or telenzepine;
a modulator of chemokine receptor function (such as a CCR1 receptor antagonist); or,
an inhibitor of p38 kinase function.

14. (canceled)

15. (canceled)

16. A method of treating a PDE 4 mediated disease state in a mammal suffering from, or at risk of, said disease, which comprises administering to a mammal in need of such treatment a therapeutically effective amount of a compound of formula (I), or a pharmaceutically acceptable salt thereof as claimed in claim 1.
